SEQUENCE LISTING (81 sequences)

SEQ ID NO: 1 - protein, 511 aa, Indiana vesiculovirus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - DNA, 1536 bp, Indiana vesiculovirus
  source 1..1536: mol_type = genomic DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 3 - protein, 265 aa, synthetic construct
  REGION 1..265: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 4 - DNA, 795 bp, synthetic construct
  misc_feature 1..795: note = Synthetic
  source 1..795: mol_type = other DNA
atggaggcaccggcgcagcttctgtttctgctgcttctctggctgccggacacaacaggccaaatcgtgctcacacagtcccctgcaatcatgagtgcctctccgggcgagaaggtaaccatgacctgcagtgcttcatcttcagtttcatatatgaactggtatcaacagaagtctggtacgtcaccaaagcggtggatttatgacacctccaaattggctagcggtgtgcctgcgcattttcgggggagtggatctgggacatcatatagtctgactataagcggaatggaagctgaggacgcggcaacttactattgccaacaatggagtagtaacccattcactttcgggagcggcactaagctggagatcaacggcgggggagggtccggaggtggtggtagtggtggcgggggatccggtggagggggcagtcaagtgcaattgcagcaatcaggagcagaacttgcacggcccggtgcttccgtgaaaatgagctgtaaagcttcaggctacacttttactcgatacacgatgcactgggtgaaacagcggccagggcagggcctggagtggattgggtatattaacccgtcacgaggatacacaaattacaatcagaaatttaaagacaaagccacgcttacaactgataagagttcttccacggcttatatgcaactgtcatccttgacttccgaggattcagcagtgtattattgtgcgcggtactatgatgaccactattgccttgattattggggacagggaacgacacttaccgtaagctct

SEQ ID NO: 5 - protein, 42 aa, synthetic construct
  REGION 1..42: note = Synthetic
KPTTTPAPRPPTPAPTIASQPLSLRPEACRPAAGGAVHTRGL

SEQ ID NO: 6 - DNA, 126 bp, synthetic construct
  misc_feature 1..126: note = Synthetic
  source 1..126: mol_type = other DNA
aagcccaccaccacccctgcccctagacctccaaccccagcccctacaatcgccagccagcccctgagcctgaggcccgaagcctgtagacctgccgctggcggagccgtgcacaccagaggcctg

SEQ ID NO: 7 - protein, 58 aa, synthetic construct
  REGION 1..58: note = Synthetic
SPKAQASSVPTAQPQAEGSLAKATTAPATTRNTGRGGEEKKKEKEKEEQEERETKTPE

SEQ ID NO: 8 - DNA, 174 bp, synthetic construct
  misc_feature 1..174: note = Synthetic
  source 1..174: mol_type = other DNA
tctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagag

SEQ ID NO: 9 - protein, 17 aa, synthetic construct
  REGION 1..17: note = Synthetic
DKTHTCPPCPAPELLGG

SEQ ID NO: 10 - DNA, 51 bp, synthetic construct
  misc_feature 1..51: note = Synthetic
  source 1..51: mol_type = other DNA
gacaaaactcacacatgcccaccgtgcccagcacctgaactcctgggggga

SEQ ID NO: 11 - protein, 776 aa, synthetic construct
  REGION 1..776: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 12 - protein, 818 aa, synthetic construct
  REGION 1..818: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSKPTTTPAPRPPTPAPTIASQPLSLRPEACRPAAGGAVHTRGLMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 13 - protein, 263 aa, synthetic construct
  REGION 1..263: note = Synthetic
MEAPAQLLFLLLLWLPDTTGDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 14 - DNA, 789 bp, synthetic construct
  misc_feature 1..789: note = Synthetic
  source 1..789: mol_type = other DNA
atggaggcacctgcacaacttctgttcttgctcttgctttggctccccgacacaacaggtgacatacagatgacacagagtccttctagtctcccagccagcctcggagacagggttacgataaattgtcaggccagtcaggacatatctaactatcttaattggtaccagcagaaaccgggtaaggcaccgaagttgctgatctattacactaacaagttggcggacggtgtgccatctagattctccggctcaggtagtgggcgcgacagcagttttactatctctagccttgaatctgaagatattgggtcatactactgccaacaatactacaactacccgtggacgttcggacctgggacaaagcttgagataaagggagggggaggaagtggcggcgggggcagtggtggaggaggatcaggcggtgggggttctgaagtacaattggttgaatctggtggaggattggtgcaaccgggcaaaagcttgaaactgtcctgcgaagctagtggttttaccttttctgggtacggaatgcattgggttcgacaagctccaggaagggggctcgaatctgttgcttatataacaagctcaagcatcaacattaaatatgcagacgcggtaaaaggacgatttacggtcagtagggataacgctaaaaacctgctgttcctccagatgaatattctcaagtcagaggatactgctatgtattattgcgctcggtttgattgggataaaaattactggggacaaggaaccatggtaaccgtctcctcc

SEQ ID NO: 15 - protein, 774 aa, synthetic construct
  REGION 1..774: note = Synthetic
MEAPAQLLFLLLLWLPDTTGDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 816 aa, synthetic construct
  REGION 1..816: note = Synthetic
MEAPAQLLFLLLLWLPDTTGDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSKPTTTPAPRPPTPAPTIASQPLSLRPEACRPAAGGAVHTRGLMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - DNA, 440 bp, synthetic construct
  misc_feature 1..440: note = Synthetic
  source 1..440: mol_type = other DNA
ctgatcagaaacaagaggatctgctaatgtggcttgtgtaatagtttctctcagtggatctaaagaggtggcacttccttgtttgtgagtaaatgtagttgtattgttcataggaagcccaaaatcttctttgtttgttaccagtgactattgctatttgttttttgttaccaggaaaggctattctaaattcccttacacttcctagaattctgaacaaactttcctcatagtctctgctttgtatcaatcatcccaaaatctttgcttcagaaagagcagtttgcacctgtttgtgttttaccccacacctagcccactgttgctttgacagtcttacaccatcaaaggcagaaggtagagaggcagatttcttctagttcctcccccactcttcctttttctgaatacccatgagtctgttggcggaggatagtgct

SEQ ID NO: 18 - protein, 90 aa, synthetic construct
  REGION 1..90: note = Synthetic
EHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - DNA, 274 bp, synthetic construct
  misc_feature 1..274: note = Synthetic
  source 1..274: mol_type = other DNA
cgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 20 - protein, 20 aa, synthetic construct
  REGION 1..20: note = Synthetic
RGPTIKPCPPCKCPAPNLLG

SEQ ID NO: 21 - DNA, 60 bp, synthetic construct
  misc_feature 1..60: note = Synthetic
  source 1..60: mol_type = other DNA
agagggcccacaatcaagccctgtcctccatgcaaatgcccagcacctaacctcttgggt

SEQ ID NO: 22 - protein, 42 aa, synthetic construct
  REGION 1..42: note = Synthetic
QASSVPTAQPQAEGSLAKATTAPATTRNTGRGGEEKKKEKEK

SEQ ID NO: 23 - DNA, 126 bp, synthetic construct
  misc_feature 1..126: note = Synthetic
  source 1..126: mol_type = other DNA
caggcctcctcagtgcccactgcacaaccccaagcagagggcagcctcgccaaggcaaccacagccccagccaccacccgtaacacaggaagaggaggagaagagaagaagaaggagaaggagaaa

SEQ ID NO: 24 - protein, 64 aa, synthetic construct
  REGION 1..64: note = Synthetic
RWPESPKAQASSVPTAQPQAEGSLAKATTAPATTRNTGRGGEEKKKEKEKEEQEERETKTPECP

SEQ ID NO: 25 - DNA, 192 bp, synthetic construct
  misc_feature 1..192: note = Synthetic
  source 1..192: mol_type = other DNA
cgctggccagagtctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccg

SEQ ID NO: 26 - protein, 5 aa, synthetic construct
  REGION 1..5: note = Synthetic
GGGGS

SEQ ID NO: 27 - protein, 10 aa, synthetic construct
  REGION 1..10: note = Synthetic
GGGGSGGGGS

SEQ ID NO: 28 - protein, 15 aa, synthetic construct
  REGION 1..15: note = Synthetic
GGGGSGGGGSGGGGS

SEQ ID NO: 29 - protein, 20 aa, synthetic construct
  REGION 1..20: note = Synthetic
GGGGSGGGGSGGGGSGGGGS

SEQ ID NO: 30 - DNA, 30 bp, synthetic construct
  misc_feature 1..30: note = Synthetic
  source 1..30: mol_type = other DNA
ggagggggaggaagtggcggcgggggcagt

SEQ ID NO: 31 - DNA, 45 bp, synthetic construct
  misc_feature 1..45: note = Synthetic
  source 1..45: mol_type = other DNA
ggagggggaggaagtggcggcgggggcagtggtggaggaggatca

SEQ ID NO: 32 - DNA, 60 bp, synthetic construct
  misc_feature 1..60: note = Synthetic
  source 1..60: mol_type = other DNA
ggagggggaggaagtggcggcgggggcagtggtggaggaggatcaggcggtgggggttct

SEQ ID NO: 33 - protein, 511 aa, synthetic construct
  REGION 1..511: note = Synthetic
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 34 - DNA, 1536 bp, synthetic construct
  misc_feature 1..1536: note = Synthetic
  source 1..1536: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 35 - protein, 511 aa, synthetic construct
  REGION 1..511: note = Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 36 - DNA, 1536 bp, synthetic construct
  misc_feature 1..1536: note = Synthetic
  source 1..1536: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 37 - protein, 511 aa, synthetic construct
  REGION 1..511: note = Synthetic
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 38 - DNA, 1536 bp, synthetic construct
  misc_feature 1..1536: note = Synthetic
  source 1..1536: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 39 - protein, 834 aa, synthetic construct
  REGION 1..834: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSPKAQASSVPTAQPQAEGSLAKATTAPATTRNTGRGGEEKKKEKEKEEQEERETKTPEMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 793 aa, synthetic construct
  REGION 1..793: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSDKTHTCPPCPAPELLGGMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 796 aa, synthetic construct
  REGION 1..796: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSRGPTIKPCPPCKCPAPNLLGMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 818 aa, synthetic construct
  REGION 1..818: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSQASSVPTAQPQAEGSLAKATTAPATTRNTGRGGEEKKKEKEKMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 840 aa, synthetic construct
  REGION 1..840: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSRWPESPKAQASSVPTAQPQAEGSLAKATTAPATTRNTGRGGEEKKKEKEKEEQEERETKTPECPMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 781 aa, synthetic construct
  REGION 1..781: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 45 - protein, 786 aa, synthetic construct
  REGION 1..786: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 791 aa, synthetic construct
  REGION 1..791: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 47 - protein, 796 aa, synthetic construct
  REGION 1..796: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 48 - protein, 786 aa, synthetic construct
  REGION 1..786: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 791 aa, synthetic construct
  REGION 1..791: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 50 - protein, 796 aa, synthetic construct
  REGION 1..796: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 51 - protein, 818 aa, synthetic construct
  REGION 1..818: note = Synthetic
MEAPAQLLFLLLLWLPDTTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSKPTTTPAPRPPTPAPTIASQPLSLRPEACRPAAGGAVHTRGLMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 52 - protein, 260 aa, synthetic construct
  REGION 1..260: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 53 - DNA, 807 bp, synthetic construct
  misc_feature 1..807: note = Synthetic
  source 1..807: mol_type = other DNA
atggaggcccccgcccaactcctcttcctcctgttgctctggctgccggacaccaccggtcaagtgcagctgcagcaatcaggggccgagctcgccaggcctggcgcatcagttaaaatgtcatgtaaggcatcagggtacacctttacaagatacacaatgcactgggtgaagcagaggcccggtcagggtctggaatggatcggctatatcaatcctagcagaggttatacaaactacaatcaaaaatttaaggataaagccacattgacaactgacaagagtagttccactgcttatatgcagctgagcagcctcacgtccgaggacagtgcagtttattattgtgcgcgatattacgacgaccattattgcttggattactggggacagggcacgacgctcacagtcagttctggcggtgggggcagtggggggggtgggtctggaggcggaggctcccagatagtcttgacgcagtcaccggctataatgtcagcaagtcctggagagaaagtcacaatgacttgcagtgcgtccagtagtgttagttacatgaactggtaccagcaaaaaagtggtacaagccccaagagatggatctacgacaccagcaagctggcaagtggtgtaccagcgcattttcgaggctctgggagtgggacgtcttactctctcacgattagcggcatggaagcggaagacgcggcaacatattactgtcaacaatggtcttccaatcctttcacttttggttcaggcacaaagctcgagataaatagagctgacacggctccgggcggcggg

SEQ ID NO: 54 - protein, 267 aa, synthetic construct
  REGION 1..267: note = Synthetic
MEAPAQLLFLLLLWLPDTTGDIVLTQTPATLSLIPGERVTMTCKTSQNIGTILHWYHQKPKEAPRALIKYASQSIPGIPSRFSGSGSETDFTLSINNLEPDDIGIYYCQQSRSWPVTFGPGTKLEIKGGGGSGGGGSGGGGSGGGGSQVKLQQSGSELGKPGASVKLSCKTSGYIFTDHYISWVKQKPGESLQWIGNVYGGNGGTSYNQKFQGKATLTVDKISSTAYMELSSLTSEDSAIYYCARRPVATGHAMDYWGQGIQVTVSS

SEQ ID NO: 55 - DNA, 801 bp, synthetic construct
  misc_feature 1..801: note = Synthetic
  source 1..801: mol_type = other DNA
atggaggcacctgcacaacttctgttcttgctcttgctttggctccccgacacaacaggtgatatagtccttactcagacccctgcaacgctttccttgatcccgggagagcgagtaactatgacctgcaagactagtcagaatatcggtacaatccttcactggtaccatcagaagcccaaagaggctcctagggcacttatcaaatatgcctctcagagtattccaggtatcccttcaagattctccgggtcagggtccgagacggactttacactttcaatcaacaatttggagccggacgatataggaatatattattgccaacaatctcggagttggcccgtcacgtttgggccaggaacaaagctcgaaataaaaggaggtggaggtagcggcggagggggatccggtggtggcggttccgggggtggtggatctcaggtaaaactccagcaatctggatcagagttgggtaaaccgggagcctccgtgaagttgtcttgtaaaacgagtgggtatatattcacggatcattacatctcctgggtgaagcaaaagccaggtgaaagtcttcagtggatcggaaacgtctatggaggtaacgggggcacgtcttataaccaaaagtttcaaggcaaggcaactttgaccgtggataagatatctagcactgcctatatggagctctcctctcttacctctgaggattcagccatttattattgtgcacgaaggccggtagctactggtcatgctatggactattgggggcagggtatacaggtaactgtatcttca

SEQ ID NO: 56 - DNA, 15 bp, synthetic construct
  misc_feature 1..15: note = Synthetic
  source 1..15: mol_type = other DNA
ggagggggaggaagt

SEQ ID NO: 57 - protein, 16 aa, synthetic construct
  REGION 1..16: note = Synthetic
MKCLLYLAFLFIGVNC

SEQ ID NO: 58 - protein, 460 aa, synthetic construct
  REGION 1..460: note = Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSS

SEQ ID NO: 59 - protein, 444 aa, synthetic construct
  REGION 1..444: note = Synthetic
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSS

SEQ ID NO: 60 - protein, 495 aa, synthetic construct
  REGION 1..495: note = Synthetic
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 61 - protein, 462 aa, synthetic construct
  REGION 1..462: note = Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWK

SEQ ID NO: 62 - protein, 464 aa, synthetic construct
  REGION 1..464: note = Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSS

SEQ ID NO: 63 - protein, 467 aa, synthetic construct
  REGION 1..467: note = Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIAS

SEQ ID NO: 64 - protein, 771 aa, synthetic construct
  REGION 1..771: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 813 aa, synthetic construct
  REGION 1..813: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINKPTTTPAPRPPTPAPTIASQPLSLRPEACRPAAGGAVHTRGLMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 829 aa, synthetic construct
  REGION 1..829: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSPKAQASSVPTAQPQAEGSLAKATTAPATTRNTGRGGEEKKKEKEKEEQEERETKTPEMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 788 aa, synthetic construct
  REGION 1..788: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINDKTHTCPPCPAPELLGGMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 791 aa, synthetic construct
  REGION 1..791: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGPTIKPCPPCKCPAPNLLGMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 813 aa, synthetic construct
  REGION 1..813: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINQASSVPTAQPQAEGSLAKATTAPATTRNTGRGGEEKKKEKEKMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 835 aa, synthetic construct
  REGION 1..835: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRWPESPKAQASSVPTAQPQAEGSLAKATTAPATTRNTGRGGEEKKKEKEKEEQEERETKTPECPMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 776 aa, synthetic construct
  REGION 1..776: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 781 aa, synthetic construct
  REGION 1..781: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - protein, 786 aa, synthetic construct
  REGION 1..786: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 791 aa, synthetic construct
  REGION 1..791: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 75 - protein, 781 aa, synthetic construct
  REGION 1..781: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 76 - protein, 786 aa, synthetic construct
  REGION 1..786: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 77 - protein, 791 aa, synthetic construct
  REGION 1..791: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSGGGGSMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 78 - protein, 813 aa, synthetic construct
  REGION 1..813: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINKPTTTPAPRPPTPAPTIASQPLSLRPEACRPAAGGAVHTRGLMKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 79 - protein, 275 aa, synthetic construct
  REGION 1..275: note = Synthetic
MEAPAQLLFLLLLWLPDTTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGS

SEQ ID NO: 80 - DNA, 825 bp, synthetic construct
  misc_feature 1..825: note = Synthetic
  source 1..825: mol_type = other DNA
atggaggcccccgcccaactcctcttcctcctgttgctctggctgccggacaccaccggtcaagtgcagctgcagcaatcaggggccgagctcgccaggcctggcgcatcagttaaaatgtcatgtaaggcatcagggtacacctttacaagatacacaatgcactgggtgaagcagaggcccggtcagggtctggaatggatcggctatatcaatcctagcagaggttatacaaactacaatcaaaaatttaaggataaagccacattgacaactgacaagagtagttccactgcttatatgcagctgagcagcctcacgtccgaggacagtgcagtttattattgtgcgcgatattacgacgaccattattgcttggattactggggacagggcacgacgctcacagtcagttctggcggtgggggcagtggggggggtgggtctggaggcggaggctcccagatagtcttgacgcagtcaccggctataatgtcagcaagtcctggagagaaagtcacaatgacttgcagtgcgtccagtagtgttagttacatgaactggtaccagcaaaaaagtggtacaagccccaagagatggatctacgacaccagcaagctggcaagtggtgtaccagcgcattttcgaggctctgggagtgggacgtcttactctctcacgattagcggcatggaagcggaagacgcggcaacatattactgtcaacaatggtcttccaatcctttcacttttggttcaggcacaaagctcgagataaatggcggtgggggcagtggggggggtgggtctggaggcggaggctcc

SEQ ID NO: 81 - protein, 5 aa, synthetic construct
  REGION 1..5: note = Synthetic
EAAAK

(19) United States
(12) Patent Application Publication
(10) Pub. No.: US 2023/0167158 A1
(43) Pub. Date: Jun. 1, 2023

(54) PSEUDOTYPED VIRUSES CONFIGURED TO EXPRESS CAR IN T-CELLS

(71) Applicants: ICHILOV TECH LTD., Tel Aviv (IL); YEDA RESEARCH AND DEVELOPMENT CO. LTD., Rehovot (IL); BOARD OF REGENTS, THE UNIVERSITY OF TEXAS SYSTEM, Austin, TX (US)

(72) Inventors: Amer M NAJJAR, Katy, TX (US); Anat GLOBERSON LEVIN, Tel Aviv (IL); Tova WAKS, Petach Tikva (IL); Galit HORN, Hod Hasharon (IL)

(21) Appl. No.: 18/096,167

(22) Filed: Jan. 12, 2023

Related U.S. Application Data
(63) Continuation of application No. PCT/IL2021/050863, filed on Jul. 14, 2021.
(60) Provisional application No. 63/051,411, filed on Jul. 14, 2020.
(60) Provisional application No. 63/075,334, filed on Sep. 8, 2020.
(60) Provisional application No. 63/180,914, filed on Apr. 28, 2021.

(51) Int. Cl. C07K 14/005; C07K 16/28; C07K 14/705; A61P 35/00

(52) U.S. Cl. CPC C07K 14/005; C07K 16/2809; C07K 14/70517; A61P 35/00; C12N 2760/20023; C07K 2317/622; C07K 2319/02; C07K 2319/03; C12N 2760/20222; C12N 2760/20242; C12N 2760/20271; A61K 2039/505

(57) ABSTRACT

The present invention discloses a fusion protein comprising a vesicular stomatitis virus envelope glycoprotein (VSVG) extracellular domain or a fragment or an analog thereof linked to a polypeptide comprising an antigen binding domain specific to cluster of differentiation 3 (CD3). The application also discloses pseudotyped viruses comprising the fusion protein and pseudotyped viruses encoding for a chimeric antigen receptor (CAR) or T-cell receptor pseudotyped in which the receptor is expressed under a CD3 promoter. Pseudotyped viruses combining these properties are encompassed as well. The application further discloses use of these pseudotyped viruses and method of producing these pseudotyped viruses.

### CROSS REFERENCE TO RELATED APPLICATIONS

This application is a ByPass Continuation of PCT Patent Application No. PCT/IL2021/050863 having International filing date of Jul. 14, 2021, which claims the benefit of priority of U.S. Provisional Patent Application No. 63/051,411, filed Jul. 14, 2020, U.S. Provisional Patent Application No. 63/075,334 filed Sep. 8, 2020, and U.S. Provisional Patent Application No. 63/180,914 filed Apr. 28, 2021 all entitled “PSEUDOTYPED VIRUSES CONFIGURED TO EXPRESS CAR IN T-CELLS”, the contents of which are all incorporated herein by reference in their entirety.

This invention was made with government support under grant number 5R01EB017206 awarded by the National Institutes of Health. The government has certain rights in the invention.

### REFERENCE TO AN ELECTRONIC SEQUENCE LISTING

The contents of the electronic sequence listing (ICH-YEDA-P-013-US.xml; Size: 143,763 bytes; and Date of Creation: Jan. 24, 2023) is herein incorporated by reference in its entirety.

### FIELD OF THE INVENTION

The present invention relates to pseudotyped viruses.

### BACKGROUND OF THE INVENTION

Immunotherapy of cancer using T cells that are reprogrammed to target tumors is a promising new approach that has culminated in effective FDA-approved treatments for relapsed or refractory B cell malignancies. Conventional adoptive T cell therapy is based on ex-vivo genetic modification and expansion of T cells to enforce the expression of chimeric antigen receptors (CARs) before reinfusion into patients. However, broader and more rapid implementation of adoptive CAR T cell therapy has been hindered by the labor-intensive, time-consuming, and expensive ex-vivo T cell modification and expansion procedures.

Several approaches have been suggested to streamline the genetic modification process by specifically targeting endogenous T cells in situ. For example, Frank and Buchholz (Molecular Therapy: Methods & Clinical Development, 2019, Vol. 12) described gene delivery into B and T cells using lentiviral vectors (LVs) pseudotyped with engineered glycoproteins recognizing lymphocyte surface markers as entry receptors. Frank and Buchholz discuss different strategies for envelope glycoprotein engineering and selection of the targeting ligand and in particular use of a CD8-targeted LV. Interestingly, Frank and Buchholz note that gene transfer into primary human T cells using CD3-coupled to murine leukemia virus (MLV) and a separate VSV molecule when used for targeting LVs had a very low efficiency. This concept was first disclosed in Maurice et al. Blood. 2002 Apr. 1; 99(7):2342-50 (PMID: 11895766; DOI: 10.1182/blood.v99.7.2342).

Yang et al (Pharm Res. 2009 June; 26(6): 1432-1445. doi:10.1007/s11095-009-9853-y) tried a parallel strategy using a lentiviral vector system constructed by co-incorporating a full-length anti-CD3 antibody (OKT3) and a fusogen (Sindbis virus, SINV) into individual viral particles.

International Patent Application WO2017/182585 discloses a retroviral vector with a targeting molecule containing Paramyxoviridae envelope glycoprotein G or a transmembrane domain and second fusogenic protein.

There is an urgent need for effective and cheap approaches to genetically modify autologous T cells for improving cancer treatment.

### SUMMARY OF THE INVENTION

The present invention provides fusion proteins comprising a vesicular stomatitis virus envelope glycoprotein (VSVG) extracellular domain or a fragment or an analog thereof linked to a polypeptide comprising an antigen-binding domain specific to cluster of differentiation 3 (CD3). Pseudotyped viruses comprising the fusion protein and pseudotyped viruses encoding a chimeric antigen receptor (CAR) or T-cell receptor (TCR) expressed under a CD3 promoter are also provided. Pseudotyped viruses combining these properties are encompassed as well. Use of these pseudotyped viruses and method of producing these pseudotyped viruses are also provided.

According to a first aspect, there is provided a pseudotyped virus or virus-like particle comprising a fusion protein comprising a vesicular stomatitis virus envelope glycoprotein (VSVG) extracellular domain (ECD) or a fragment or an analog thereof capable of fusing with a cellular membrane linked to a polypeptide comprising an antigen binding domain specific to cluster of differentiation 3 (CD3).

According to another aspect, there is provided a fusion protein comprising a vesicular stomatitis virus envelope glycoprotein (VSVG) extracellular domain or a fragment or an analog thereof capable of fusing with a cellular membrane linked at its N-terminus to a polypeptide comprising an antigen binding domain specific to cluster of differentiation 3 (CD3).

According to another aspect, there is provided a nucleic acid molecule encoding a fusion protein of the invention.

According to another aspect, there is provided a pharmaceutical composition comprising a pseudotyped virus of the invention, and a pharmaceutically acceptable carrier, excipient or adjuvant.

According to another aspect, there is provided a method of treating cancer in a subject in need thereof comprising administering a therapeutically effective amount of a pseudotyped virus of the invention, or a pharmaceutical composition comprising of the invention to the subject, thereby treating cancer.

According to some embodiments, the polypeptide is a single variable fragment (scFv) of an antibody that specifically binds CD3.

According to some embodiments, the scFv is OKT3.

According to some embodiments, the ECD comprises or consists of SEQ ID NO: 59.

According to some embodiments, the pseudotyped virus comprises full-length VSVG.

According to some embodiments, the full-length VSVG comprises a signal peptide and comprises or consists of SEQ ID NO: 1 or wherein the full-length VSVG is devoid of a signal peptide and comprises or consists of SEQ ID NO: 60.

According to some embodiments, the pseudotyped virus comprises a truncated VSVG lacking an intracellular domain.

According to some embodiments, the fusion protein further comprises a transmembrane domain from a protein other than VSVG.

According to some embodiments, the pseudotyped virus or virus-like particle of the invention further comprises a truncated VSVG comprising or consisting of amino acid sequence SEQ ID NO: 18.

According to some embodiments, the VSVG comprises a mutation that decreases binding to low-density lipoprotein (LDL) receptor.

According to some embodiments, the mutation is selected from mutation of K47 of VSVG, mutation of R354 of VSVG or both, optionally wherein the K47 is mutated to A, G or Q, the R354 is mutated to A or G or wherein the VSVG analog comprises or consists of an amino acid sequence selected from SEQ ID NO: 33, 35 and 37.

According to some embodiments, the polypeptide is linked to an N-terminus of the VSVG ECD, fragment or analog thereof.

According to some embodiments, the VSVG, analog or fragment thereof and the polypeptide are linked via a linker.

According to some embodiments, the linker is a peptide linker.

According to some embodiments, the peptide linker comprises at least 10 amino acids.

According to some embodiments, the linker is selected from a CD8a stalk, an IgG hinge, an IgD hinge, an IgD linker, an IgG2a linker, a helical linker, a proline-rich linker and a GGGGS linker, wherein the GCGCS linker comprises 2 to 5 repetitions of amino acid sequence GGGGS or wherein the linker comprises or consists of an amino acid sequence selected from SEQ ID NO: 5, 7, 9, 20, 22, 24, and 26-29.

According to some embodiments, the linker is a rigid linker.

According to some embodiments, the fusion protein comprises or consists of an amino acid sequence selected from SEQ ID NO: 11, 12, 39-51 and 64-78.

According to some embodiments, the fusion protein comprises or consists of an amino acid sequence selected from SEQ ID NO: 12, 39-51 and 65-78.

According to some embodiments, the virus is selected from lentivirus, adenovirus, retrovirus, Epstein-Barr virus, herpes simplex virus 1 (HSV1), a myxoma virus, a reovirus, a poliovirus, a vesicular stomatitis virus (VSV), and a measles virus (MV).

According to some embodiments, the pseudotyped virus or virus-like particle further comprises a membranal protein of interest or a nucleic acid molecule encoding for the membranal protein of interest.

According to some embodiments, the membranal protein of interest is a chimeric antigen receptor (CAR) or a T-cell receptor.

According to some embodiments, the membranal protein of interest is a CAR and the CAR binds specifically to a tumor-associated antigen.

According to some embodiments, the tumor associated antigen is selected from ErbB2/Her2, CD19, CD20, CD22, CD30, CD33, CD38, CD40, CD123, CD133, CD138, CD5, CD7, APRIL, BCMA, CEA, MUCI, EGFR, GD2, Mesothelin, and CDK4.

According to some embodiments, the pseudotyped virus or virus-like particle of the invention comprises a nucleic acid molecule encoding the membranal protein of interest.

According to some embodiments, the pseudotyped virus or virus-like particle of the invention comprises a trimer comprising said fusion protein.

According to some embodiments, the nucleic acid molecule comprises regulatory element operably linked to an open reading frame encoding the membranal protein of interest and wherein the regulatory element induces transcription in T cells.

According to some embodiments, the regulatory element is a T cell active promoter and is selected from a CD3, CD4 and CD8 promoter.

According to some embodiments, the promoter is a CD3 promoter.

According to some embodiments, the fusion protein comprises full-length VSVG.

According to some embodiments, the full-length VSVG comprises a signal peptide and comprises or consists of SEQ ID NO: 1 or wherein the full-length VSVG is devoid of a signal peptide and comprises or consists of SEQ ID NO: 60.

According to some embodiments, the VSVG is a truncated VSVG comprising or consisting of amino acid sequence SEQ ID NO: 18.

According to some embodiments, the VSVG, analog or fragment thereof and the polypeptide are linked via a linker.

According to some embodiments, the linker is a peptide linker.

According to some embodiments, the peptide linker comprises at least 10 amino acids.

According to some embodiments, the linker is a rigid linker.

According to some embodiments, the linker is selected from a CD8a stalk, an IgG hinge, an IgD linker and a GGGGS linker, wherein the GGGGS linker comprises 2 to 5 repetitions of amino acid sequence GGGGS or wherein the linker comprises or consists of an amino acid sequence selected from SEQ ID NO: 5, 7, 9, 20, 22, 24, and 26-29.

According to some embodiments, the fusion protein of the invention comprises an amino acid sequence selected from SEQ ID NO: 11, 12, 39-51 and 64-78.

According to some embodiments, the fusion protein of the invention consists of an amino acid sequence selected from SEQ ID NO: 11, 12, 39-51 and 64-78.

According to some embodiments, the fusion protein of the invention comprises an amino acid sequence selected from SEQ ID NO: 12, 39-51 and 65-78.

According to some embodiments, the fusion protein of the invention consists of an amino acid sequence selected from SEQ ID NO: 12, 39-51 and 65-78.

According to some embodiments, the fusion protein is in a form of a homotrimer.

According to some embodiments, the pharmaceutical composition of the invention is for use in treating cancer.

According to some embodiments, the administering is systemically or intratumorally administering.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-C: Schematic illustrations of the CD3-specific pseudotype fusion proteins. (1A) Pre-(green) and post-(magenta) fusion monomer structures of VSVG are superimposed. The fusion domains of both structures are shown in yellow. Each VSVG monomer undergoes a dramatic structural transition from the pre-infusion to the post-infusion configuration where the fusion domain relocates 1600 from its original position.

Inclusion of a linker between the single chain variable fragments (scFv) and the N-terminal domain of VSVG is necessary to minimize steric hindrance during the receptor structural transition states. Furthermore, the linker distances the scFv domains from VSVG monomers to allow their unhindered trimerization at their interface sites. (1B) Anti-CD3 (aCD3) scFv containing a single chain or V_{L} and V_{H} fused by a flexible linker is fused with VSVG or truncated VSVG composed of amino acids 421-512 (VTMD). All pseudotyping receptors are anchored to the cell membrane via the transmembrane domain of VSVG.

Several linkers have been inserted between the scFv and VSVG regions to minimize steric hindrances and improve CD3-binding affinity. (1C) Composite structure of pseudotyping receptor illustrates the fusion of VSVG (orange) with the anti-CD3 scFv (V_{L} (grey) and V_{H}(blue)). A domain of CD3ε (brown) is shown engaging the scFv and a portion of the LDL receptor (pink) is also shown bound to its cognate site on VSVG.

FIG. 2: Bioluminescence image of mouse splenocytes and human peripheral mononuclear cells (PBMC) in a plate showing specific mouse lymphocyte transduction with a lentivirus pseudotyped with receptor comprised of a mouse CD3-specific scFv (derived from antibody 2C11) fused with VSVG (2C11-VSVG) and encoding firefly luciferase (ffLuc). Mouse splenocytes and human PBMCs were transfected with 2C11-VSVG and VSVG-psuedotyped lentivectors expressing ffLuc. The VSVG pseudoytpes did not exhibit cell specificity as indicated by ffLuc expression in both human and mouse cells. The 2C11-VSVG pseudotype, however, exhibited specificity for mouse splenocytes with no detectable luciferase activity in human PBMC.

FIGS. 3A-I: Structural modifications to improve CD3-binding affinity and specificity. (3A) Composite structural depiction of CD3-VSVG pseudotyping receptor illustrating the insertion of a linker sequence to minimize steric hindrances between the VSVG and CD3 scFv domains. K47 and R354 residues denoted by red arrows are optionally mutated to eliminate VSVG binding to its native receptor (the LDL receptor) to improve CD3-specificity of the chimeric pseudotyping receptor. (3B-C) Histograms showing improvement of CD3 binding through inclusion of a CD8a stalk linker in 293 cells transfected with mouse CD3-Fc and contacted with (3B) 2C11-VSVG and (3C) 2C11-CD8a-VSVG expressing virus. (3D-F) Histograms of GFP positive cells within a lymphocyte population transduced with virus containing envelope protein (3D) VSVG alone, (3E) OKT3-VSVG and (3F) OKT3-CD8a-VSVG. (3G-I) Histograms of GFP positive cells within a lymphocyte population transduced with virus containing an anti-HER2 CAR as well as (3G) VSVG alone, (3H) OKT3-VSVG and (3I) OKT3-CD8a-VSVG. Untransduced (UT) cells were used as control.

FIGS. 4A-B: CD3 promoter-driven expression of GFP in human lymphocytes. Human PBMCs were transduced with lentiviral vectors expressing GFP under the control of a (4A) CMV or (4B) human CD3 promoter. GFP expression is detected only from the CD3 promoter vector in human lymphocytes.

FIGS. 5A-C: Comparison of GFP expression following in situ transduction. MLV vs. VSVG envelope and CD3 promoter were tested for specific expression in lymphocytes. (5A-C) Representative histograms of FACS analysis of GFP expression in CD3 positive and negative cells from the blood of mice in situ transduced with (5A) PBS control, (5B) VSVG and CD3 promoter-GFP and (5C) MLV and CD3 promoter-GFP.

FIGS. 6A-B: Expression and effect of CARs directed against HER2 (4D5 antibody) and under control of the CD3 promoter after IV administration. (6A) Bioluminescence images of mice showing the CAR is expressed primarily in the spleen and bone marrow of mice after in situ transduction with MLV enveloped virus when under CD3 promoter regulation. (6B) Line graph of tumor progression after intra-tumoral injection to tghuHER2 tumor bearing mice of mixed splenocytes that were activated in vitro (yellow line) or were mixed with virus containing the Her2 CAR under the control of the CD3 promoter (purple line), or activated lymphocytes infected with GFP expressing virus (blue line) or activated lymphocytes infected with the Her2 CAR virus (red line). Mice treated with virus containing only GFP was used as a negative control. 2×10⁶ splenocytes or 100 μl of lentiparticles were administered per injection.

FIGS. 7A-B: In situ transduction with VSVG and 2C11-VSVG pseudotyped lentivirus. (7A) Bioluminescence imaging of VSVG pseudotyped lentivirus expressing ffLuc-GFP was injected intravenously and monitored by bioluminescence imaging to determine transduction pattern. Bioluminescence coinciding with the liver was detected at day 3 and intensified systemically by day 7. (7B) Bioluminescence imaging of in situ transduction with a 2C11-VSVG vector yielding a bioluminescence signal pattern corresponding to the skeletal system and consistent with localization of T cells.

### DETAILED DESCRIPTION OF THE INVENTION

The present invention is based on the surprising finding that a fusion protein comprising an N-terminal anti-CD3 scFv and a C-terminal VSVG polypeptide separated by a peptide linker can produce virus particles that not only target and infect T-cells but also have reduced binding to the native cellular target receptor of VSVG, the LDL receptor. These viruses also facilitate the generation of CAR T-cells in vivo, abrogating the need for T cell extraction and ex vivo manipulation. This in vivo CAR-T generation was effective at reducing tumor size and was indeed comparable to adoptive T cell transfer with in vitro activation of the T cells.

It was further found that a virus encoding for CAR expression under control of the CD3 promoter produced expression predominantly in lymphatic organs indicating transduction of only T-cells. This approach may be used to generate CAR T-cell as a stand-alone method or in combination with a VSVG pseudotyped virus targeting CD3. The combination of the two methods greatly reduces off-target effects and restricts delivery exclusively to T cells. The described methods may provide an efficient off-the-shelf treatment to cancer patients.

By a first aspect, there is provided a fusion protein comprising a vesicular stomatitis virus envelope glycoprotein (VSVG) or a fragment or an analog thereof and a polypeptide comprising an antigen binding domain capable of binding to cluster of differentiation 3 (CD3).

By another aspect, there is provided a pseudotyped virus or virus-like particle comprising a fusion protein comprising a vesicular stomatitis virus envelope glycoprotein (VSVG) or a fragment or an analog thereof and a polypeptide comprising an antigen binding domain capable of binding to cluster of differentiation 3 (CD3).

By another aspect, there is provided a pseudotyped virus or virus-like particle comprising a fusion protein of the invention.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “fusion protein” refers to a single polypeptide chain that contains domains or moieties from two distinct proteins that do not appear in a single polypeptide chain in nature. In some embodiments, the fusion protein is a chimeric protein. In some embodiments, the fusion protein is an artificial protein. In some embodiments, the fusion protein is not found in nature. The fusion protein may be formed by the joining of two or more peptides through a peptide bond formed between the amino-terminus of one peptide and the carboxyl-terminus of another peptide. The fusion protein may be expressed as a single polypeptide fusion protein from a nucleic acid sequence encoding the single contiguous conjugate. In some embodiments, fusion proteins are created through the joining of two or more genes that originally coded for separate proteins. Recombinant fusion proteins may be created artificially by recombinant DNA technology for use in biological research or therapeutics. “Chimeric” or “chimera” usually designate hybrid proteins made of polypeptides having different functions or physicochemical patterns. For example, a fusion protein can comprise a first part that is a CD3 binding fragment, and a second part (e.g., genetically fused to the first part) that comprises a VSVG extracellular domain (e.g., the full length VSVG). Methods of fusion protein generation, recombinant protein generation, recombinant DNA generation, and DNA fusion techniques are well known in the art, and any such method for making the chimeric molecules of the invention may be employed.

As used herein, the term “recombinant protein” refers to a protein which is coded for by a recombinant nucleic acid molecule (DNA or RNA) and is thus not naturally occurring. The term “recombinant DNA or RNA” refers to DNA or RNA molecules formed by laboratory methods of genetic recombination. Generally, this recombinant molecule is in the form of an mRNA, a vector, a plasmid or a virus, used to express the recombinant protein in a cell.

VSVG is a viral envelope glycoprotein. In some embodiments, VSVG is the envelope protein of vesicular stomatitis virus (VSV). In some embodiments, VSVG is the envelope protein of vesicular stomatitis Indiana virus. In some embodiments, vesicular stomatitis is vesicular stomatitis Indiana virus. In some embodiments, VSVG is the glycoprotein of VSV. In some embodiments, the amino acid sequence of VSVG is provided in Uniprot number P03522. According to some embodiments, the VSVG protein comprises the amino acid sequence provided in SEQ ID NO:1. In some embodiments, the VSVG protein consists of SEQ ID NO: 1. In some embodiments, the VSVG protein is encoded by the nucleic acid sequence provided in SEQ ID NO: 2. In some embodiments, VSVG comprises a signal domain (SP). In some embodiments, SEQ ID NO: 1 comprises the SP. In some embodiments, VSVG is devoid of an SP. In some embodiments, VSVG devoid of an SP. In some embodiments, the VSVG SP comprises SEQ ID NO: 57. In some embodiments, the VSVG SP consists of SEQ ID NO: 57. In some embodiments, the VSVG SP comprises amino acids 1-16 of SEQ ID NO: 1. In some embodiments, the VSVG SP consists of amino acids 1-16 of SEQ ID NO: 1. In some embodiments, VSVG is full-length VSVG. In some embodiments, full-length VSVG comprises SEQ ID NO: 1. In some embodiments, full-length VSVG consists of SEQ ID NO: 1. In some embodiments, full-length VSVG is devoid of an SP. In some embodiments, full-length VSVG devoid of an SP comprises SEQ ID NO: 60. In some embodiments, full-length VSVG devoid of an SP consists of SEQ ID NO: 60.

In some embodiments, VSVG comprises an SP, an extracellular domain (ECD), a transmembrane domain and an intracellular domain. In some embodiments, VSVG comprises an ECD a transmembrane domain and an intracellular domain. In some embodiments, the VSVG or a fragment or an analog thereof comprises an ECD of VSVG. In some embodiments, the ECD is the full ECD. In some embodiments, the ECD is a fragment of the ECD capable of fusion. The exact amino acids that constitute the transmembrane domain of VSVG is not consistently agreed upon in the literature. As such, the exact sequence of the VSVG ECD is also not agreed upon in that the C-terminal end is said sometimes to include more or fewer amino acids. In some embodiments, the ECD comprises SEQ ID NO: 58. In some embodiments, the ECD consists of SEQ ID NO: 58. In some embodiments, the ECD ends in WFSS. In some embodiments, the ECD comprises SEQ ID NO: 61. In some embodiments, the ECD consists of SEQ ID NO: 61. In some embodiments, the ECD ends in SSWK. In some embodiments, the ECD comprises SEQ ID NO: 62. In some embodiments, the ECD consists of SEQ ID NO: 62. In some embodiments, the ECD ends in WKSS. In some embodiments, the ECD comprises SEQ ID NO: 63. In some embodiments, the ECD consists of SEQ ID NO: 63. In some embodiments, the ECD ends in SSIAS. In some embodiments, the ECD is devoid of an SP. In some embodiments, the ECD devoid of an SP comprises SEQ ID NO: 59. In some embodiments, the ECD devoid of an SP consists of SEQ ID NO: 59. In some embodiments, the ECD comprises amino acids 1 to 467 of SEQ ID NO: 1. In some embodiments, the ECD consists of amino acids 1 to 467 of SEQ ID NO: 1. In some embodiments, the ECD devoid of an SP comprises amino acids 17 to 467 of SEQ ID NO: 1. In some embodiments, the ECD devoid of an SP consists of amino acids 17 to 467 of SEQ ID NO: 1. In some embodiments, the ECD comprises amino acids 1 to 460 of SEQ ID NO: 1. In some embodiments, the ECD consists of amino acids 1 to 460 of SEQ ID NO: 1. In some embodiments, the ECD devoid of an SP comprises amino acids 17 to 460 of SEQ ID NO: 1. In some embodiments, the ECD devoid of an SP consists of amino acids 17 to 460 of SEQ ID NO: 1. In some embodiments, the ECD comprises amino acids 1 to 462 of SEQ ID NO: 1. In some embodiments, the ECD consists of amino acids 1 to 462 of SEQ ID NO: 1. In some embodiments, the ECD devoid of an SP comprises amino acids 17 to 462 of SEQ ID NO: 1. In some embodiments, the ECD devoid of an SP consists of amino acids 17 to 462 of SEQ ID NO: 1. In some embodiments, the ECD comprises amino acids 1 to 464 of SEQ ID NO: 1. In some embodiments, the ECD consists of amino acids 1 to 464 of SEQ ID NO: 1. In some embodiments, the ECD devoid of an SP comprises amino acids 17 to 464 of SEQ ID NO: 1. In some embodiments, the ECD devoid of an SP consists of amino acids 17 to 464 of SEQ ID NO: 1. In some embodiments, the ECD comprises amino acids 1 to 467 of SEQ ID NO: 1. In some embodiments, the ECD consists of amino acids 1 to 467 of SEQ ID NO: 1. In some embodiments, the ECD devoid of an SP comprises amino acids 17 to 467 of SEQ ID NO: 1. In some embodiments, the ECD devoid of an SP consists of amino acids 17 to 467 of SEQ ID NO: 1.

In some embodiments, the fusion protein comprises a transmembrane domain. In some embodiments, the transmembrane domain comprises the VSVG transmembrane domain. In some embodiments, the transmembrane domain is the VSVG transmembrane domain. In some embodiments, the VSVG transmembrane domain comprises amino acids 461 to 483 of SEQ ID NO: 1. In some embodiments, the VSVG transmembrane domain consists of amino acids 461 to 483 of SEQ ID NO: 1. In some embodiments, the VSVG transmembrane domain comprises amino acids 461 to 488 of SEQ ID NO: 1. In some embodiments, the VSVG transmembrane domain consists of amino acids 461 to 488 of SEQ ID NO: 1. In some embodiments, the VSVG transmembrane domain comprises amino acids 461 to 489 of SEQ ID NO: 1. In some embodiments, the VSVG transmembrane domain consists of amino acids 461 to 489 of SEQ ID NO: 1. In some embodiments, the VSVG transmembrane domain comprises amino acids 463 to 483 of SEQ ID NO: 1. In some embodiments, the VSVG transmembrane domain consists of amino acids 463 to 483 of SEQ ID NO: 1. In some embodiments, the VSVG transmembrane domain comprises amino acids 463 to 488 of SEQ ID NO: 1. In some embodiments, the VSVG transmembrane domain consists of amino acids 463 to 488 of SEQ ID NO: 1. In some embodiments, the VSVG transmembrane domain comprises amino acids 463 to 489 of SEQ ID NO: 1. In some embodiments, the VSVG transmembrane domain consists of amino acids 463 to 489 of SEQ ID NO: 1. In some embodiments, the VSVG transmembrane domain comprises amino acids 465 to 483 of SEQ ID NO: 1. In some embodiments, the VSVG transmembrane domain consists of amino acids 465 to 483 of SEQ ID NO: 1. In some embodiments, the VSVG transmembrane domain comprises amino acids 465 to 488 of SEQ ID NO: 1. In some embodiments, the VSVG transmembrane domain consists of amino acids 465 to 488 of SEQ ID NO: 1. In some embodiments, the VSVG transmembrane domain comprises amino acids 465 to 489 of SEQ ID NO: 1. In some embodiments, the VSVG transmembrane domain consists of amino acids 465 to 489 of SEQ ID NO: 1. In some embodiments, the VSVG transmembrane domain comprises amino acids 468 to 483 of SEQ ID NO: 1. In some embodiments, the VSVG transmembrane domain consists of amino acids 468 to 483 of SEQ ID NO: 1. In some embodiments, the VSVG transmembrane domain comprises amino acids 468 to 488 of SEQ ID NO: 1. In some embodiments, the VSVG transmembrane domain consists of amino acids 468 to 488 of SEQ ID NO: 1. In some embodiments, the VSVG transmembrane domain comprises amino acids 468 to 489 of SEQ ID NO: 1. In some embodiments, the VSVG transmembrane domain consists of amino acids 468 to 489 of SEQ ID NO: 1.

In some embodiments, the transmembrane domain is a non-VSVG transmembrane domain. In some embodiments, the non-VSVG transmembrane domain is a transmembrane domain of a viral protein. In some embodiments, the viral protein is a viral membrane protein. In some embodiments, the viral protein is a viral envelope protein. In some embodiments, the viral protein is a viral protein capable of fusion. In some embodiments, the non-VSVG transmembrane domain is a mammalian transmembrane domain. In some embodiments, the mammal is a human. The term “transmembrane domain” refers to a generally hydrophobic region which crosses or bridges a lipid membrane. The transmembrane domain may be any naturally-occurring or non-naturally occurring transmembrane domain. In some embodiments, the transmembrane domain is a naturally occurring transmembrane domain.

The transmembrane domain may be a transmembrane domain of a receptor, a transmembrane protein, preferably a viral transmembrane protein, a fragment of a transmembrane protein, a transmembrane peptide or a variant thereof, such as a genetically modified transmembrane domain of a receptor, a genetically modified transmembrane protein, a genetically modified fragment of a transmembrane protein or a genetically modified transmembrane peptide. In some embodiments, the transmembrane domain is a transmembrane domain of a receptor. In some embodiment, the transmembrane domain is a viral transmembrane domain.

Examples of transmembrane domains include, but are not limited to, the transmembrane domain (TMD) of the platelet-derived growth factor receptor (PDGFR), the transmembrane domain of CD34 or the VSVG transmembrane domain. The N-terminus of the transmembrane domain is preferably fused, directly or indirectly (for example via a linker), to the C-terminus of the VSVG ECD.

In some embodiments, the fusion protein comprises an intracellular domain. In some embodiments, the intracellular domain is the VSVG intracellular domain. The term “intracellular domain” refers to the intracellular part of a transmembrane protein or a receptor.

In some embodiments, the fragment comprises the VSVG ECD and transmembrane domain. In some embodiments, the fragment consists of the VSVG ECD and transmembrane domain. In some embodiments, the fragment comprises amino acids 1 to 483 of SEQ ID NO: 1. In some embodiments, the fragment consists of amino acids 1 to 483 of SEQ ID NO: 1. In some embodiments, the fragment comprises amino acids 1 to 488 of SEQ ID NO: 1. In some embodiments, the fragment consists of amino acids 1 to 488 of SEQ ID NO: 1. In some embodiments, the fragment comprises amino acids 1 to 489 of SEQ ID NO: 1. In some embodiments, the fragment consists of amino acids 1 to 489 of SEQ ID NO: 1.

In some embodiments, the VSVG or a fragment or an analog thereof is capable of fusion. In some embodiments, capable of fusion is capable of fusion to a cellular membrane. In some embodiments, the cellular membrane is a plasma membrane. In some embodiments, the cellular membrane is an intracellular membrane. In some embodiments, the cellular membrane is an endosomal membrane. In some embodiments, the cellular membrane is a lysosomal membrane.

In some embodiments, a fragment is a fragment comprising the ECD. In some embodiments, a fragment is a fragment capable of fusion. In some embodiments, a fragment comprises at least 5, 10, 15, 20, 25, 30, 35, 40, 45, 50, 55, 60, 65, 70, 75, 80, 85, 90, 95, 100, 125, 150, 200, 250, 300, 350, 400, 450 or 500 amino acids. Each possibility represents a separate embodiment of the invention. In some embodiments, a fragment comprises at most 50, 100, 150, 200, 250, 300, 350, 400, 450 or 500 amino acids. Each possibility represents a separate embodiment of the invention.

According to another embodiments, the fusion protein comprises an analog of VSVG. The term “analog” refers to a polypeptide, peptide or protein which differs by one or more amino acid alterations (e.g., substitutions, additions or deletions of amino acid residues) from the original sequence, or which comprises an additional chemical modification that does not alter the amino acid sequence. In some embodiments, the analog is an analog comprising at least 70, 75, 80, 85, 90, 95, 97, 98 or 99% sequence identity to the original sequence. In some embodiments, the original sequence is SEQ ID NO: 1. In some embodiments, the original sequence is SEQ ID NO: 58. In some embodiments, the original sequence is SEQ ID NO: 59. In some embodiments, the original sequence is SEQ ID NO: 60. In some embodiments, the analog is an analog capable of fusion. In some embodiments, an analog still maintains the properties of the parent polypeptide, peptide or protein. According to one embodiment, the analog comprises at least one modification selected from a substitution, deletion and addition. According to some embodiments, the modification is a substitution. In some embodiments, the modification is a mutation. In some embodiments, the mutation is a substitution. According to one embodiment, the substitution is a conservative substitution. In some embodiments, the substitution decreases binding of VSVG to its canonical receptor. In some embodiments, the canonical receptor is LDLR. In some embodiments, the substitution abolishes binding of VSVG to its canonical receptor.

According to some embodiments, the VSVG or a fragment or an analog thereof is as described in any one of the aspects and embodiments of the present invention. According to some embodiments, the VSVG fragment is a truncated VSVG. According to some embodiments, the VSVG analog comprises a mutation selected from mutation at K47, R354 or both. In some embodiments, the amino acids number is with respect to SEQ ID NO: 1. It will be understood that if no SP is included in the VSVG than K47 will be K31 and R354 will be R338. According to some embodiments, the analog of VSVG comprises a mutation of K47. According to some embodiments, the analog of VSVG comprises a mutation of R354. In some embodiments, the analog of VSVG comprises mutation of K47 and R354. In some embodiments, K47 is mutated to A. In some embodiments, K47 is mutated to G. In some embodiments, K47 is mutated to Q. In some embodiments, K47 is mutated to any one of A, G and Q. In some embodiments, R354 is mutated to A. In some embodiments, R354 is mutated to G. In some embodiments, R354 is mutated to any one of A and G. In some embodiments, the mutation is selected from K47A, K47G, K47Q R354A, and R347G. According to some embodiments, the analog of VSVG comprises K47Q/R354A mutations. According to some embodiments, the analog comprises amino acid sequence selected from SEQ ID NOs: 33, 35 and 37. According to some embodiments, the analog consists of amino acid sequence selected from SEQ ID NOs: 33, 35 and 37. According to some embodiments, the analog comprises SEQ ID NO: 33. According to some embodiments, the analog consists of SEQ ID NO: 33. According to some embodiments, the analog comprises SEQ ID NO: 35. According to some embodiments, the analog consists of SEQ ID NO: 35. According to some embodiments, the analog comprises SEQ ID NO: 37. According to some embodiments, the analog consists of SEQ ID NO: 37.

According to some embodiments, the fragment of VSVG comprises the amino acids sequence of SEQ ID NO: 18. According to some embodiments, the fragment of VSVG consists of the amino acids sequence of SEQ ID NO. 18. According to some embodiments, the fusion protein comprises a fragment of VSVG, i.e., a truncated VSVG. According to some embodiments, the fragment of VSVG comprises amino acids sequence SEQ ID NO: 18, comprising 39 amino acids of the extracellular domain, 23 of the transmembrane domain, and 28 of the cytoplasmic domain of the original VSVG. It will be understood by a skilled artisan that this breakdown of the domains in not wholly agreed upon in the literature as discussed hereinabove.

According to any one of the above embodiments, the VSVG, an analog or a fragment thereof and the polypeptide are linked via a linker. The term “linker” relates to any peptide capable of connecting the VSVG, an analog or a fragment thereof and the polypeptide and subsequently reduces steric hindrance.

According to some embodiments, the polypeptide comprising an antigen binding domain capable of binding CD3. In some embodiments, the CD3 is human CD3. In some embodiments, the CD3 is mammalian CD3. In some embodiments, the CD3 is murine CD3. In some embodiments, the CD3 is CD3e. In some embodiments, the antigen biding domain specifically binds to a CD3. In some embodiments, the antigen binding domain is an antibody. In some embodiments, the antigen binding domain is a fragment of an antibody.

As used herein, the term “antibody” refers to immunoglobulins comprising two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (Fragment crystalline) domains. The antigen binding domains, Fab, include regions where the polypeptide sequence varies. The term F (ab′)2 represents two Fab′ arms linked together by disulfide bonds. The central axis of the antibody is termed the Fc fragment. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CH1). The variable domains of each pair of light and heavy chains form the antigen-binding site. The domains of the light and heavy chains have the same general structure, and each domain comprises four framework regions, whose sequences are relatively conserved, joined by three hyper-variable domains known as complementarity determining regions (CDRs). These domains contribute specificity and affinity of the antigen-binding site. The isotype of the heavy chain (gamma, alpha, delta, epsilon or mu) determines immunoglobulin class (IgG, IgA, IgD, IgE or IgM, respectively). The light chain is either of two isotypes (kappa (κ) or lambda (λ)) found in all antibody classes.

The terms “antibody fragment” as used herein refers to only a portion of an intact antibody, generally including an antigen-binding site of the intact antibody and thus retaining the ability to bind antigen. The term refers to the antibody as well as to the analog or variant of said antibody. In some embodiments, the term “fragment” and “antibody fragment” are used interchangeably. Examples of antibody fragment encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 1988, 242, 423-426; and Huston et al., PNAS (USA) 1988, 85, 5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

According to some embodiments, the antibody fragment is a single chain fragment being a composite polypeptide having antigen binding capabilities and comprising amino acid sequences homologous or analogous to the variable regions of an immunoglobulin light and heavy chain i.e., linked VH-VL or single chain Fv (scFv). The VH and VL domains in the scFv may be in any order. Thus, according to some embodiments, the scFv is VH-VL or VL-VH. In some embodiments, the scFv is configured VH-VL. In some embodiments, the scFv is configured VL-VH. In some embodiments, the VH and VL are connected by a linker. In some embodiments, the linker is a flexible linker. In some embodiments, the antigen binding domain is an scFv.

The term antibody fragment encompasses also darpins. The term “darpin”, as used herein, refers to a genetically engineered antibody mimetic protein typically exhibiting highly specific and high-affinity target protein binding. It is derived from natural ankyrin proteins and consists of at least three, usually four or five repeat motifs of these proteins.

According to some embodiments, the terms “antibody” or “antibodies” collectively refer to intact antibodies, i.e., monoclonal antibodies (mAbs), analogs and variant thereof, as well as proteolytic fragments thereof, such as the Fab or F(ab′)2 fragments and scFv.

According to some embodiments, the polypeptide is a single variable fragment (scFv) of an antibody that binds specifically to CD3. ScFvs that bind to CD3 are well known in the art and include for example OKT3 and 500A2. Any such molecule may be employed. According to some embodiments, the antibody that specifically binds human CD3 is OKT3 antibody. According to some embodiments, the scFv of OKT3 comprises or consists of the amino acid sequence of SEQ ID NO: 3. According to some embodiments, the scFv of OKT3 comprises or consists of the amino acid sequence of SEQ ID NO: 53. Thus, according to some embodiments, the fusion protein comprising a vesicular stomatitis virus envelope glycoprotein (VSVG) or a fragment or an analog thereof linked to an scFv. According to one embodiment, the fusion protein comprises VSVG fused to the scFv OKT3 and comprises the amino acid sequence of SEQ ID NO: 11. According to one embodiment, the fusion protein comprises VSVG fused to the scFv OKT3 and comprises the amino acid sequence of SEQ ID NO: 64. According to one embodiment, the fusion protein comprises VSVG fused to the scFv OKT3 and consists of the amino acid sequence of SEQ ID NO: 11. According to one embodiment, the fusion protein comprises VSVG fused to the scFv OKT3 and consists of the amino acid sequence of SEQ ID NO: 64.

As described above, the VH and VL domains in the scFv may be in any order. Thus, according to some embodiments, the scFv of OKT3 is a VL-VH ordered scFv having SEQ ID NO: 3 or 56. According to other embodiments, the scFv of OKT3 is a VL-VH ordered scFv SEQ ID NO: 3. According to other embodiments, the scFv of OKT3 is a VL-VH ordered scFv SEQ ID NO: 56. According to other embodiments, the scFv of OKT3 is a VH-VL ordered scFv SEQ ID NO: 52 or 79. According to other embodiments, the scFv of OKT3 is a VH-VL ordered scFv SEQ ID NO: 52. According to other embodiments, the scFv of OKT3 is a VH-VL ordered scFv SEQ ID NO: 79. According to some embodiments, SEQ ID NO: 79 is encoded by SEQ ID NO: 80. Thus, according to any one of the embodiments and aspects of the preset invention it is contemplated that an amino acid sequence corresponding to SEQ ID NO: 3 within the fusion protein may be substituted with amino acid sequence SEQ ID NO: 52. Each of such sequences in which SEQ ID NO: 3 is substituted with SEQ ID NO: 52 present a separate embodiment. Thus, according to any one of the embodiments and aspects of the preset invention it is contemplated that an amino acid sequence corresponding to SEQ ID NO: 56 within the fusion protein may be substituted with amino acid sequence SEQ ID NO: 79. Each of such sequences in which SEQ ID NO: 56 is substituted with SEQ ID NO: 79 present a separate embodiment. According to some embodiments, the VH and VL domains (in either order) of scFv of OKT3 are linked by a spacer comprising 1 to 6 repetition of the amino acid sequence GGGGS. In some embodiments, the spacer comprises 1 repetition of GGGGS. In some embodiments, the spacer comprises 2 repetitions of GGGGS. In some embodiments, the spacer comprises 3 repetitions of GGGGS. In some embodiments, the spacer comprises 4 repetitions of GGGGS. In some embodiments, the spacer comprises 5 repetitions of GGGGS. In some embodiments, the spacer comprises 6 repetitions of GGGGS.

According to other embodiments, the antibody that specifically binds CD3 is selected from SK7, UCHT1 and CD3-12. According to some embodiments, the polypeptide is a scFv of any one of SK7, UCHT1 and CD3-12.

The terms “binds specifically” or “specific for” with respect to an antigen-binding domain of an antibody, of a fragment thereof or of a CAR refers to an antigen-binding domain which recognizes and binds to a specific antigen but does not substantially recognize or bind other molecules. The term encompasses that the antigen-binding domain binds to its antigen with high affinity and binds other antigens with low affinity. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

In some embodiments, the VSVG or a fragment or analog thereof is linked to the polypeptide. In some embodiments, linked is linked by a linker. In some embodiments, the linker is a polypeptide linker. In some embodiments, the linker is a peptide bond. In some embodiments, the linker is a non-amino acid linker. In some embodiments, the linker comprises at least one amino acid. In some embodiments, the linker comprises a plurality of amino acids. In some embodiments, the linker comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. Each possibility represents a separate embodiment of the invention. In some embodiments, the linker comprises at least 10 amino acids. In some embodiments, the linker comprises at least 16 amino acids. In some embodiments, the linker comprises at least 40 amino acids. In some embodiments, the linker comprises at least 42 amino acids. In some embodiments, the linker comprises at least 55 amino acids. In some embodiments, the linker comprises at least 58 amino acids. In some embodiments, the linker comprises at most 10, 15, 20, 25, 30, 35, 40, 45, 50, 55, 60, 65, 70, 75, 80, 85, 90, 95, 100, 105 110, 115, 120, 125, 130, 135, 140, 145, 150, 160, 170, 180, 190, or 200 amino acids. Each possibility represents a separate embodiment of the invention. In some embodiments, the linker is a flexible linker. In some embodiments, the linker comprises a signal peptide of VSVG. In some embodiments, the linker length is the amino acid length in addition to the signal peptide.

Thus, according to some embodiments, the fusion protein has a structure of a (polypeptide)-(linker)-(VSVG an analog or fragment thereof). According to some embodiments, the polypeptide is an scFv of an antibody that specifically binds to CD3, e.g., scFv of OKT3 antibody.

In some embodiments, the linker is a rigid linker. Rigid linkers are well known in the art and have a stiff/non-flexible structure. Rigid linkers can have helical or non-helical confirmations. Non-helical linkers tend to be proline-rich, as this residue contributes to the rigidity due to its cyclic side chain, that restricts rotational freedom. Further, the lack of an amide hydrogen eliminates secondary hydrogen bond interactions with neighboring residues. For example, the linker between the lipoyl and E3 binding domains of pyruvate dehydrogenase is characteristically rigid due to its proline-rich sequence and can serve as effectively linker between scFv and VSVG. Amino acid sequences that adopt α-helical conformations can form rigid linkers due to the secondary hydrogen bonds that stabilize and closely pack the structure. Moreover, these α-helical conformations form rapidly, minimizing their interaction with neighboring domains during the folding process.

In some embodiments, a rigid linker is a helical linker. In some embodiments, helical is alpha-helical. In some embodiments, a rigid linker is selected from a helical linker and proline-rich linker. In some embodiments, proline-rich comprises at least 10, 15, 20, 25, 30, 35, 40, 45, or 50% proline. Each possibility represents a separate embodiment of the invention. In some embodiments, proline-rich comprises at least 50% proline. In some embodiments, proline-rich comprises at least 10% proline. In some embodiments, proline-rich comprises at least 25% proline. Examples of rigid linkers found in naturally occurring proteins include, but are not limited to, the IgG hinge, the IgD hinge, the CD8a stalk, the IgG2a linker, and the IgD linker. In some embodiments, the rigid linker is selected from a proline-rich linker, an alpha-helical linker, an IgD hinge, an IgG hinge, a CD8a stalk, an IgG2a linker, and an IgD linker.

In some embodiments, the rigid linker is a proline-rich linker. In some embodiments, a proline-rich linker comprises or consists of the amino acid sequence (XP)n. In some embodiments, n is an integer. In some embodiments, n is an integer from 1-10, 1-9, 1-8, 1-7, 1-6, 1-5, 1-4, 2-10, 2-9, 2-8, 2-7, 2-6, 2-5, 2-4, 3-10, 3-9, 3-8, 3-7, 3-6, 3-5, 3-4, 4-10, 4-9, 4-8, 4-7, 4-6, 4-5, 5-10, 5-9, 5-8, 5-7 or 5-6. Each possibility represents a separate embodiment of the invention. In some embodiments, n is an integer from 1-10. In some embodiments, X is any amino acid. In some embodiments, X is any amino acid other than proline. In some embodiments, X is selected from A, K and E. In some embodiments, X is A. In some embodiments, X is K. In some embodiments, X is E.

In some embodiments, the rigid linker is a helical linker. In some embodiments, the helical linker is an alpha-helical linker. In some embodiments, an alpha-helical linker is an α-helical linker. In some embodiments, a helical linker comprises the amino acid sequence EAAAK (SEQ ID NO: 81). In some embodiments, a helical linker consists of SEQ ID NO: 81. In some embodiments, the helical linker comprises (EAAAK)n wherein n is an integer number of repeats of SEQ ID NO: 81. In some embodiments, n is an integer from 1-10, 1-9, 1-8, 1-7, 1-6, 1-5, 1-4, 2-10, 2-9, 2-8, 2-7, 2-6, 2-5, 2-4, 3-10, 3-9, 3-8, 3-7, 3-6, 3-5, 3-4, 4-10, 4-9, 4-8, 4-7, 4-6, 4-5, 5-10, 5-9, 5-8, 5-7 or 5-6. Each possibility represents a separate embodiment of the invention. In some embodiments, n is an integer from 1-10.

In some embodiments, the rigid linker is derived from IgD. In some embodiments, the rigid linker is derived from the IgD hinge region. In some embodiments, a linker derived from IgD or the IgD hinge is an IgD linker. In some embodiments, the rigid linker is the IgD hinge. In some embodiments, the IgD hinge comprises SEQ ID NO: 7. In some embodiments, the IgD hinge consists of SEQ ID NO: 7. In some embodiments, the IgD linker comprises SEQ ID NO: 22. In some embodiments, the IgD linker consists of SEQ ID NO: 22. In some embodiments, the IgD linker comprises SEQ ID NO: 24. In some embodiments, the IgD linker consists of SEQ ID NO: 24.

In some embodiments, the rigid linker is derived from IgG. In some embodiments, the rigid linker is derived from the IgG hinge. In some embodiments, the rigid linker is the IgG hinge. In some embodiments, IgG is any one of IgG1, IgG2, IgG3 and IgG4. In some embodiments, IgG is IgG1. In some embodiments, the IgG1 hinge comprises SEQ ID NO: 9. In some embodiments, the IgG hinge consists of SEQ ID NO: 9. In some embodiments, the IgG is IgG2. In some embodiments, IgG2 is IgG2a. In some embodiments, the IgG2a linker comprises SEQ ID NO: 20. In some embodiments, the IgG2a linker consists of SEQ ID NO: 20.

In some embodiments, the rigid linker is derived from CD8. In some embodiments, CD8 is CD8a. In some embodiments, the rigid linker is derived from the CD8 hinge. In some embodiments, the CD8a linker comprises SEQ ID NO: 5. In some embodiments, the CD8a linker consists of SEQ ID NO: 5.

According to some embodiment, the linker comprises an amino acid sequence of CD8a stalk. According to one embodiment, the CD8a stalk has amino acid sequence SEQ ID NO: 5. According to some embodiment, the linker comprises amino acid sequence of an IgG hinge. In some embodiments, the IgG is IgG1. In some embodiments, the IgG is IgG2. In some embodiments, IgG2 is IgG2a. In some embodiments, the IgG is IgG 3. In some embodiments, the IgG is IgG 4. In some embodiments, the linker is an IgD linker. In some embodiments, the linker is an IgD-based linker. In some embodiments, the IgD linker is an IgD hinge. According to some embodiment, the IgD hinge comprises or consists of the amino acid sequence of SEQ ID NO: 7. In some embodiment, the IgD comprises or consists of the amino acid sequence of SEQ ID NO: 22. In some embodiment, the IgD comprises or consists of the amino acid sequence of SEQ ID NO: 24. In some embodiments, the IgG1 hinge comprises or consists of SEQ ID NO: 9. In some embodiments, the IgG2a hinge comprises or consists of SEQ ID NO: 20. In some embodiments, the flexible linker is a glycine-serine (GS) linker. According to one embodiment, the linker comprises or consists of 2-5 repeats of the GGGGS sequence (SEQ ID NO: 26). In some embodiments, the GS linker comprises or consists of SEQ ID NO: 27. In some embodiments, the GS linker comprises or consists of SEQ ID NO: 28. In some embodiments, the GS linker comprises or consists of SEQ ID NO: 29. According to some embodiments, the linker has an amino acid sequence selected from SEQ ID NO: 5, 7, 9, 20, 22, 24, and 26-29.

According to one embodiment, the fusion protein comprises amino acid sequence SEQ ID NO: 11. According to one embodiment, the fusion protein consists amino acid sequence SEQ ID NO: 11. According to one embodiment, the fusion protein comprises amino acid sequence SEQ ID NO: 12. According to one embodiment, the fusion protein consists amino acid sequence SEQ ID NO: 12. According to one embodiment, the fusion protein comprises amino acid sequence SEQ ID NO: 39. According to one embodiment, the fusion protein consists amino acid sequence SEQ ID NO: 39. According to one embodiment, the fusion protein comprises amino acid sequence SEQ ID NO: 40. According to one embodiment, the fusion protein consists amino acid sequence SEQ ID NO: 40. According to one embodiment, the fusion protein comprises amino acid sequence SEQ ID NO: 41. According to one embodiment, the fusion protein consists amino acid sequence SEQ ID NO: 41. According to one embodiment, the fusion protein comprises amino acid sequence SEQ ID NO: 42. According to one embodiment, the fusion protein consists amino acid sequence SEQ ID NO: 42. According to one embodiment, the fusion protein comprises amino acid sequence SEQ ID NO: 43. According to one embodiment, the fusion protein consists amino acid sequence SEQ ID NO: 43. According to one embodiment, the fusion protein comprises amino acid sequence SEQ ID NO: 44. According to one embodiment, the fusion protein consists amino acid sequence SEQ ID NO: 44. According to one embodiment, the fusion protein comprises amino acid sequence SEQ ID NO: 45. According to one embodiment, the fusion protein consists amino acid sequence SEQ ID NO: 45. According to one embodiment, the fusion protein comprises amino acid sequence SEQ ID NO: 46. According to one embodiment, the fusion protein consists amino acid sequence SEQ ID NO: 46. According to one embodiment, the fusion protein comprises amino acid sequence SEQ ID NO: 47. According to one embodiment, the fusion protein consists amino acid sequence SEQ ID NO: 47. According to one embodiment, the fusion protein comprises amino acid sequence SEQ ID NO: 48. According to one embodiment, the fusion protein consists amino acid sequence SEQ ID NO: 48. According to one embodiment, the fusion protein comprises amino acid sequence SEQ ID NO: 49. According to one embodiment, the fusion protein consists amino acid sequence SEQ ID NO: 49. According to one embodiment, the fusion protein comprises amino acid sequence SEQ ID NO: 50. According to one embodiment, the fusion protein consists amino acid sequence SEQ ID NO: 50. According to one embodiment, the fusion protein comprises amino acid sequence SEQ ID NO: 51. According to one embodiment, the fusion protein consists amino acid sequence SEQ ID NO: 51. According to other embodiments, the fusion protein comprises amino acid sequence selected from SEQ ID Nos: 39-51. According to other embodiments, the fusion protein consists of an amino acid sequence selected from SEQ ID Nos: 39-51. According to other embodiments, the fusion protein comprises amino acid sequence selected from SEQ ID Nos: 12 and 39-51. According to other embodiments, the fusion protein consists of amino acid sequence selected from SEQ ID Nos: 12 and 39-51. According to other embodiments, the fusion protein comprises amino acid sequence selected from SEQ ID Nos: 11, 12 and 39-51. According to other embodiments, the fusion protein consists of amino acid sequence selected from SEQ ID Nos: 11, 12 and 39-51.

According to one embodiment, the fusion protein comprises amino acid sequence SEQ ID NO: 64. According to one embodiment, the fusion protein consists amino acid sequence SEQ ID NO: 64. According to one embodiment, the fusion protein comprises amino acid sequence SEQ ID NO: 65. According to one embodiment, the fusion protein consists amino acid sequence SEQ ID NO: 65. According to one embodiment, the fusion protein comprises amino acid sequence SEQ ID NO: 66. According to one embodiment, the fusion protein consists amino acid sequence SEQ ID NO: 66. According to one embodiment, the fusion protein comprises amino acid sequence SEQ ID NO: 67. According to one embodiment, the fusion protein consists amino acid sequence SEQ ID NO: 67. According to one embodiment, the fusion protein comprises amino acid sequence SEQ ID NO: 68. According to one embodiment, the fusion protein consists amino acid sequence SEQ ID NO: 68. According to one embodiment, the fusion protein comprises amino acid sequence SEQ ID NO: 69. According to one embodiment, the fusion protein consists amino acid sequence SEQ ID NO: 69. According to one embodiment, the fusion protein comprises amino acid sequence SEQ ID NO: 70. According to one embodiment, the fusion protein consists amino acid sequence SEQ ID NO: 70. According to one embodiment, the fusion protein comprises amino acid sequence SEQ ID NO: 71. According to one embodiment, the fusion protein consists amino acid sequence SEQ ID NO: 71. According to one embodiment, the fusion protein comprises amino acid sequence SEQ ID NO: 72. According to one embodiment, the fusion protein consists amino acid sequence SEQ ID NO: 72. According to one embodiment, the fusion protein comprises amino acid sequence SEQ ID NO: 73. According to one embodiment, the fusion protein consists amino acid sequence SEQ ID NO: 73. According to one embodiment, the fusion protein comprises amino acid sequence SEQ ID NO: 74. According to one embodiment, the fusion protein consists amino acid sequence SEQ ID NO: 74. According to one embodiment, the fusion protein comprises amino acid sequence SEQ ID NO: 75. According to one embodiment, the fusion protein consists amino acid sequence SEQ ID NO: 75. According to one embodiment, the fusion protein comprises amino acid sequence SEQ ID NO: 76. According to one embodiment, the fusion protein consists amino acid sequence SEQ ID NO: 76. According to one embodiment, the fusion protein comprises amino acid sequence SEQ ID NO: 77. According to one embodiment, the fusion protein consists amino acid sequence SEQ ID NO: 77. According to one embodiment, the fusion protein comprises amino acid sequence SEQ ID NO: 78. According to one embodiment, the fusion protein consists amino acid sequence SEQ ID NO: 78. According to other embodiments, the fusion protein comprises amino acid sequence selected from SEQ ID Nos: 66-78. According to other embodiments, the fusion protein consists of an amino acid sequence selected from SEQ ID Nos: 66-78. According to other embodiments, the fusion protein comprises amino acid sequence selected from SEQ ID Nos: 65-78. According to other embodiments, the fusion protein consists of amino acid sequence selected from SEQ ID Nos: 65-78. According to other embodiments, the fusion protein comprises amino acid sequence selected from SEQ ID Nos: 64-78. According to other embodiments, the fusion protein consists of amino acid sequence selected from SEQ ID Nos: 64-78. According to other embodiments, the fusion protein comprises an amino acid sequence selected from SEQ ID Nos: 39-51 and 66-78. According to other embodiments, the fusion protein consists of an amino acid sequence selected from SEQ ID Nos: 39-51 and 66-78. According to other embodiments, the fusion protein comprises amino acid sequence selected from SEQ ID Nos: 12, 39-51 and 65-78. According to other embodiments, the fusion protein consists of amino acid sequence selected from SEQ ID Nos: 12, 39-51 and 65-78. According to other embodiments, the fusion protein comprises amino acid sequence selected from SEQ ID Nos: 11-12, 39-51 and 64-78. According to other embodiments, the fusion protein consists of amino acid sequence selected from SEQ ID Nos: 11-12, 39-51 and 64-78.

In some embodiments, the polypeptide is linked to the N-terminus of the VSVG or fragment or analog thereof. In some embodiments, the C-terminus of the polypeptide is linked to the N-terminus of the VSVG or fragment or analog thereof. In some embodiments, the linker linked the C-terminus of the polypeptide to the N-terminus of the VSVG or fragment or analog thereof. In some embodiments, an SP is linked to the N-terminus of the polypeptide. In some embodiments, the SP is the SP of VSVG. It will be understood that the SP will allow the fusion protein to be translated into the ER and expressed on a plasma membrane. In some embodiments, the fusion protein is devoid of an N-terminal SP as it enters a viral particle and does not need to enter the ER.

In some embodiments, the fusion protein is in the form or a trimer. In some embodiments, the trimer is a homotrimer. In some embodiments, the trimer is a heterotrimer. In some embodiments, all three peptides of the trimer are fusion proteins of the invention. In some embodiments, at least two of the peptides of the trimer are fusion proteins of the invention. In some embodiments, at least one of the peptides of the trimer are fusion proteins of the invention. In some embodiments, the timer is a heterotrimer comprising two different fusion proteins of the invention. In some embodiments, one of the two different fusion proteins is a fusion protein comprising a truncated VSVG extracellular domain.

According to another aspect, there is provided a trimeric protein complex comprising a fusion protein of the invention.

The term “pseudotyped virus” as used herein refers to a virus or viral particle comprising a heterologous envelope protein and that was not present in the original envelope of the virus, i.e., derived from at least another virus than the original virus and/or being an engineered envelope glycoprotein, for example a chimeric and/or mutated envelope protein such as a glycoprotein. A pseudotyped virus can be generated by a complementation cell line comprising polynucleotides sufficient for packaging of the pseudotyped virus. In some embodiments, a pseudotyped virus is generated by expressing an envelope protein in a cell. In some embodiments, a pseudotyped virus is generated by expressing a gag-pol protein in a cell. In some embodiments, a pseudotyped virus is generated by expressing a reverse transcriptase in a cell. In some embodiments, the cell is a cell of a cell line. In some embodiments, the cell is a cell of a cell line. In some embodiments, the cell line is a packaging cell line. Packaging cell lines are cell lines that have high expression and are robust in culture. Cells such as 293 and 293T can be used among many others known in the art.

In some embodiments, the expressing is by expressing a nucleic acid molecule encoding the virus protein. In some embodiments, the nucleic acid molecule is a vector. In some embodiments, the vector is an expression vector. In some embodiments, the expression vector is configured to express in the packaging cell line. In some embodiments, a separate vector contains a coding sequence encoding each viral protein. In some embodiments, a vector contains at least two coding sequences each encoding a viral protein.

The term “expression” as used herein refers to the biosynthesis of a gene product, including the transcription and/or translation of that gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene/protein within a cell is well known to one skilled in the art. It can be carried out by, among many methods, transfection, viral infection, or direct alteration of the cell's genome. In some embodiments, the gene is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the promoter is configured for expression in a cell. In some embodiments, the promoter is configured for expression in a cell of a packaging cell line. In some embodiments, the promoter is configured for expression in a T cell. In some embodiments, the promoter is a CD3 promoter.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the pseudotyped virus is a virus. In some embodiments, the pseudotyped virus is a virion. In some embodiment, the pseudotyped virus is a virus-like particle (VLP). In some embodiments, the virus is a retrovirus. In some embodiments, the virus is a lentivirus. In some embodiments, the VLP is a retrovirus-like particle. In some embodiments, the VLP is a lentivirus-like particle. As used herein, a “viral-like particle” refers to a particle comprising viral proteins, but lacking virus genetic information. In some embodiments, the viral protein is an envelope protein. In some embodiments, the viral protein is a Gag protein. In some embodiments, the viral protein is a polymerase protein.

According to any one of the above embodiments, the virus is a virus having a lipid bilayer envelope. In some embodiments, the virus is a DNA virus. In some embodiments, the virus is an RNA virus. In some embodiments, the virus is selected form an RNA and a DNA virus. According to some embodiments, the virus is selected from lentivirus, adenovirus, Epstein-Barr virus, retrovirus, herpes simplex virus 1 (HSV1), a myxoma virus, a reovirus, a poliovirus, a vesicular stomatitis virus (VSV), and a measles virus (MV). In one particular embodiment, the vector is lentivirus. In some embodiments, the virus is a retrovirus. In some embodiments, the virus is an adenovirus. Retrovirus is a member of the Retroviridae family. According to some embodiments, the retrovirus may be an Oncovirus, Lentivirus or Spumavirus. According to any one of the above embodiments, the virus is a Coronavirus. According to some embodiments, the Coronavirus is selected from SARS such as SARS-CoV and SARS-CoV-2, and MERS-CoV.

According to some embodiments, the virus suitable for use in particular embodiments contemplated herein include, but are not limited to an adenovirus, a herpes simplex virus 1 (HSV1), a myxoma virus, a reovirus, a poliovirus, a vesicular stomatitis virus (VSV), a measles virus (MV), a lassa virus (LASV), or a Newcastle disease virus (NDV). Any virus can be attenuated by the methods disclosed herein. The virus can be a dsDNA virus (e.g. Adenoviruses, Herpesviruses, Poxviruses), a single stranded “plus” sense DNA virus (e.g., Parvoviruses) a double stranded RNA virus (e.g., Reoviruses), a single stranded+sense RNA virus (e.g. Picornaviruses, Togaviruses), a single stranded “minus” sense RNA virus (e.g. Orthomyxoviruses, Rhabdoviruses), a single stranded+sense RNA virus with a DNA intermediate (e.g. Retroviruses), or a double stranded reverse transcribing virus (e.g. Hepadnaviruses). In certain non-limiting embodiments of the present invention, the virus is poliovirus (PV), rhinovirus, influenza virus including avian flu (e.g. H5N1 subtype of influenza A virus), severe acute respiratory syndrome (SARS) coronavirus, Human Immunodeficiency Virus (HIV), Hepatitis B Virus (HBV), Hepatitis C Virus (HCV), infectious bronchitis virus, ebolavirus, Marburg virus, dengue fever virus (Flavivirus serotypes), West Nile disease virus, Epstein-Barr virus (EBV), yellow fever virus, Ebola (ebolavirus), chickenpox (varicella-zoster virus), measles (a paramyxovirus), mumps (a paramyxovirus), rabies (Lyssavirus), human papillomavirus, Kaposi's sarcoma-associated herpesvirus, Herpes Simplex Virus (HSV Type 1), or genital herpes (HSV Type 2). According to some embodiments, the vector is a virus selected from lentivirus, adenovirus, modified adenovirus and retrovirus. The Vesicular Stomatitis Virus (VSV) is a species of the genus Vesiculovirus within the family Rhabdoviridae within the order Mononegavirales. The genome of VSV encodes for the G protein that is responsible for binding and entry of the virus into the target cell. It is a homotrimer that induces clathrin-mediated endocytosis in the endosome once the receptor has been bound on the cell surface. In the endosome, the pH shift induces a conformational change of the homotrimer inducing irreversible fusion of the viral and cellular membrane. In one particular embodiment, the vector is lentivirus.

According to some embodiments, the pseudotyped virus is lentivirus comprising the fusion protein of the present invention.

According to some embodiments, the pseudotyped virus or virus-like particle further comprises a molecule of interest. In some embodiments, the molecule of interest is a protein of interest. In some embodiments, the molecule of interest is a nucleic acid molecule of interest. In some embodiments, the nucleic acid molecule is a vector. In some embodiments, the nucleic acid molecule encodes the protein of interest. In some embodiments, the protein of interest is a membranal protein. In some embodiments, the membranal protein is a receptor. In some embodiments, the nucleic acid molecule of interest is a regulatory nucleic acid. In some embodiments, the regulatory nucleic acid is a regulatory RNA. Regulatory RNAs are well known in the art and include, but are not limited to, siRNAs, shRNAs, lncRNAs, miRNAs and the like. In some embodiments, the regulatory nucleic acid molecule is a DNA that encodes a regulatory RNA. In some embodiments, the regulatory nucleic acid molecule is an antisense oligonucleotide (ASO). Antisense oligonucleotides are well known in the art and are used to target a variety or transcripts and/or treat a variety of conditions. In some embodiments, the ASO comprises a modified backbone. In some embodiments, modified is chemically modified.

In some embodiments, the the pseudotyped virus or virus-like particle further comprises a truncated VSVG. In some embodiments, the truncated VSVG is a truncated VSVG described hereinabove. In some embodiments, the truncated VSVG comprises SEQ ID NO: 18. In some embodiments, the truncated VSVG consists of SEQ ID NO: 18.

According to any one of the above embodiments, the pseudotyped virus of the present invention encodes for a T-cell receptor. In some embodiments, the protein of interest is a T-cell receptor. In some embodiments, the molecule of interest is a nucleic acid molecule encoding a T-cell receptor.

According to any one of the above embodiments, the pseudotyped virus of the present invention encodes for a chimeric antigen receptor (CAR). In some embodiments, the protein of interest is a CAR. In some embodiments, the molecule of interest is a nucleic acid molecule encoding a CAR. In some embodiments, the molecule of interest increases T cell cytotoxicity. In some embodiments, the molecule of interest increases specific T cell killing. In some embodiments, specific T cell killing is specific killing of a cancer cell. In some embodiments, the cancer is a tumor. In some embodiments, the CAR binds to a tumor-specific antigen. In some embodiments, the binds are binds specifically.

As used herein, the terms “chimeric antigen receptor” or “CAR” are used interchangeably and refer to an engineered recombinant polypeptide or receptor which can be grafted onto cells and comprises at least (1) an extracellular domain comprising an antigen-binding region, e.g., a single chain variable fragment of an antibody or a whole antibody, (2) a transmembrane domain to anchor the CAR into a cell, and (3) one or more cytoplasmic signaling domains (also referred to herein as “an intracellular signaling domains”). In some embodiments, the CAR comprises an ECD comprising an antigen binding region. In some embodiments, the antigen-binding region binds to a tumor-specific antigen. In some embodiments, the extracellular domain comprises an antigen binding domain (ABD) and optionally a spacer or hinge region. The antigen binding domain of the CAR targets a specific antigen. The targeting regions may comprise full length heavy chain, Fab fragments, or single chain variable fragment (scFvs). The antigen binding domain can be derived from the same species or a different species for or in which the CAR will be used in. In one embodiment, the antigen binding domain is an scFv.

In some embodiments, the extracellular domain comprises a spacer region. In some embodiments, the spacer is a hinge region. In some embodiments, an extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, constant domains such as CH2 region or CH3 region of antibodies, accessory proteins, artificial spacer sequences or combinations thereof. In some embodiments, the hinge is a stalk domain. In some embodiments, the hinge is from an immune receptor. Immune receptors are well known in the art and include for example CD8, CD4 and CD28. In some embodiments, the immune receptor is CD8. In some embodiments, the immune receptor is CD28.

In some embodiments, the CAR comprises a transmembrane domain. In some embodiments, the transmembrane domain is a CAR transmembrane domain. In some embodiments, the transmembrane domain is a transmembrane domain of an immune receptor.

As used herein, the terms “antigen binding portion” and “antigen binding domain” are used herein interchangeable and refer to one or more fragments of an antibody that retain the ability to specifically bind to an antigen. It has been shown that the antigen binding function of an antibody can be performed by fragments of a full-length antibody.

Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb, which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). Such single chain antibodies are also intended to be encompassed within the term “antigen binding portion” of an antibody.

In one embodiment of the invention, the CAR includes a transmembrane domain being a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment of the invention, the CAR comprises a costimulatory domain, e.g., a costimulatory domain comprising a functional signaling domain of a protein selected from the group consisting of OX40, CD2, CD27, CD28, CD5, ICAM-1, LFA-1 (CD11a/CD18), ICOS (CD278), and 4-1BB (CD137). In certain embodiments of the invention, the CAR comprises an scFv. According to some embodiments, the CAR binds specifically to a tumor associated antigen. The term “tumor antigen” as used herein includes both tumors associated antigens (TAAs) and tumor specific antigens (TSAs). A tumor associated antigen means an antigen that is expressed on the surface of a tumor cell in higher amounts than is observed on normal cells or an antigen that is expressed on normal cells during fetal development. The term “tumor specific antigen” refers an antigen that is unique to tumor cells and is not expressed on normal cells. The term tumor antigen includes TAAs or TSAs that have been already identified and those that have yet to be identified and includes fragments, epitopes and any and all modifications to the tumor antigens.

According to one embodiment, the CAR binds specifically to a tumor associated antigen. According to some embodiments, the tumor associated antigen is selected from AFP, ALK, APRIL, B7H3, BAGE protein, BCMA, BIRC5, BIRC7, β-catenin, -8 brc-abl, BRCA1, BORIS, CA9, CA125, carbonic anhydrase IX, caspasel, CALR, CCR5, CD5, CD7, CD19, CD20, CD22, CD30, CD33, CD38, CD40, CD123, CD133, CD138, CDK4, CEA, Claudin 18.2, cyclin-B1, CYP1B1, EGFR, EGFRvIII, ErbB2/Her2, ErbB3, ErbB4, ETV6-AML, EpCAM, EphA2, Fra-1, FOLR1, GAGE, GD2, GD3, GloboH, phosphatidylinositol proteoglycan-3, GM3, gp100, Her2, HLA/B-raf-kinases, HLA/k-ras, HLA/MAGE-A3, hTERT, IL13R a2. LMP2 k-Light, LewisY, MAGE, MART-1, Mesothelin, ML-IAP, MOv-γ, Mucd, Muc2, Muc3, Muc4, Muc5, CA-125, MUM1, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA, RAGE protein, Ras, RGS5, Rho, ROR1, SART-1, SART-3, STEAP1, STEAP2, TAG-72, TGF-β, TMPRSS2, soup-antigen, TRP-1, TRP-2, tyrosinase, urea soluble protein-3 and 5T4.

In some embodiments, the pseudotyped virus encodes for a CAR that binds specifically to a tumor associated antigen is selected from CD19, CD20, CD22, CD30, CD33, CD38, CD40, CD123, CD133, CD138, CDK4, and ErbB2/Her2. In some embodiments, the pseudotyped virus encodes for a CAR that binds specifically to a tumor associated antigen is selected from CD19, CD20, CD22, CD30, CD33, CD38, CD40, CD123, CD133, CD138, CDK4, CD5, CD7, APRIL, BCMA, CEA, MUCI, EGFR, GD2, Mesothelin, and ErbB2/Her2.

In some embodiments, the molecule of interest is a nucleic acid molecule encoding the CAR. In some embodiments, the nucleic acid molecule comprises an open reading frame encoding the molecule of interest. In some embodiments, open reading frame is operatively linked to a regulatory element. In some embodiments, the regulatory element is a promoter. The terms “operably linked” and “operatively linked” are used interchangeably and are intended to mean that the nucleotide sequence of interest is linked to the regulatory element or elements in a manner that allows for expression of the nucleotide sequence (e.g. in an in vitro transcription/translation system or in a host cell when the vector is introduced into the host cell). In some embodiments, the regulatory element is a cell-specific element. In some embodiments, the cell is a T cell. In some embodiments, the regulatory element induces transcription in T cells. In some embodiments, the regulatory element is configured to induce transcription in T cells. In some embodiments, the regulatory element is an activator. In some embodiments, the promoter is active in T cells. In some embodiments, the promoter is specific to T cells. Examples of promoters active in T cells include the CD3 promoter, the CD4 promoter and the CD8 promoter, among many others. In some embodiments, the promoter is the CD3 promoter. According to some embodiments, the cell-specific promoter is selected from a CD3, CD4 and CD8 promoter.

According to another aspect, the present invention provides a pseudotyped virus comprising a nucleic acid encoding for a receptor selected from a chimeric antigen receptor (CAR) and a T-cell receptor, wherein the nucleic acid encoding for the receptor is operably linked to a cell-specific promoter. Any definitions and embodiments of the previous aspects apply herein as well. According to some embodiments, the cell-specific promoter is selected from a CD3, CD4 and CD8 promoter. According to one embodiment, the promoter is CD3 promoter. According to some embodiments, the CD3 promoter has nucleic acid sequence SEQ ID NO: 17. According to some embodiments, the receptor is CAR as described in any of the aspects and embodiments, of the present invention. According to some embodiments, the CAR specifically binds to a tumor associated antigen selected from CD19, CD20, CD22, CD30, CD33, CD38, CD40, CD123, CD133, CD138, CDK4, and ErbB2/Her2. According to some embodiments, the CAR specifically binds to a tumor associated antigen selected from CD19, CD20, CD22, CD30, CD33, CD38, CD40, CD123, CD133, CD138, CDK4, CD5, CD7, APRIL, BCMA, CEA, MUCI, EGFR, GD2, Mesothelin, and ErbB2/Her2.

According to some embodiments, the polypeptide is a single variable fragment (scFv) of an antibody that specifically binds CD3. According to some embodiments, the polypeptide is scFv of OKT3. According to some embodiments, the scFv of OKT3 has amino acid sequence SEQ ID NO: 3. According to some embodiments, the scFv of OKT3 has amino acid sequence SEQ ID NO: 52.

According to some embodiments, the fusion protein comprises the VSVG, an analog or a fragment thereof and the polypeptide linked via a linker. According to some embodiments, the linker is as defined hereinabove. According to one embodiment, the linker comprises at least 10 amino acids. According to some embodiments, the linker has amino acid sequence of CD8a stalk. According to some embodiments, the linker has amino acid sequence of IgG hinge or IgD link or the linker is GCGC linker. According to some embodiments, the linker has amino acid sequence selected from SEQ ID NO: 5, 7, 9, 20, 22, 24, and 26-29.

According to some embodiments, the fusion protein comprises amino acid sequence SEQ ID NO: 11. According to some embodiments, the fusion protein comprises amino acid sequence SEQ ID NO: 64. According to other embodiments, the fusion protein comprises amino acid sequence SEQ ID NO: 12. According to other embodiments, the fusion protein comprises amino acid sequence SEQ ID NO: 65. According to other embodiments, the fusion protein comprises amino acid sequence selected from SEQ ID NOs: 39-51. According to other embodiments, the fusion protein comprises amino acid sequence selected from SEQ ID NOs: 66-78.

In some embodiments, the pseudotyped virus comprises a trimer of a fusion protein of the invention. In some embodiments, the pseudotyped virus comprises a timer comprising a fusion protein of the invention. In some embodiments, the pseudotyped virus comprises a trimeric form of the fusion protein of the invention. In some embodiments, the trimer is a homotrimer. In some embodiments, the trimer is a heterotrimer. In some embodiments, the trimer is a timer or trimeric complex of the invention.

According to some embodiments, the present invention provides use of the fusion protein of the present invention for preparation of a pseudotyped virus.

According to some embodiments, the present invention provides a virus comprising the fusion protein of the present invention.

According to another aspect, there is provided a nucleic acid molecule encoding a fusion protein of the invention.

According to another aspect, the present invention provides a pharmaceutical composition comprising the pseudotyped virus or virus-like particle of the present invention.

In some embodiments, the composition further comprises a pharmaceutically acceptable carrier, excipient or adjuvant.

According to some embodiments, the present invention provides a provides a pharmaceutical composition comprising a pseudotyped virus comprising an nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3). According to some embodiments, the fusion protein comprises amino acid sequence SEQ ID NO: 11. According to some embodiments, the fusion protein comprises amino acid sequence SEQ ID NO: 64. According to other embodiments, the fusion protein comprises amino acid sequence SEQ ID NO: 12. According to other embodiments, the fusion protein comprises amino acid sequence SEQ ID NO: 65.

According to other embodiments, the fusion protein comprises amino acid sequence selected from SEQ ID NOs: 39-51. According to other embodiments, the fusion protein comprises amino acid sequence selected from SEQ ID NOs: 66-78.

According to some embodiments, the present invention provides a provides a pharmaceutical composition comprising a pseudotyped virus comprising an nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3), and wherein the nucleic acid encoding for a receptor is operably linked to a cell-specific promoter. According to one embodiment, the cell-specific promoter is selected from CD3, CD4 and CD8 promoter. According to another embodiment, the cell-specific promoter is CD3 promoter.

According to some embodiments, the present invention provides a provides a pharmaceutical composition comprising a pseudotyped virus comprising a nucleic acid encoding for a receptor selected from a chimeric antigen receptor (CAR) and T-cell receptor, wherein the nucleic acid encoding for the receptor is operably linked to a cell-specific promoter. According to one embodiment, the cell-specific promoter is selected from CD3, CD4 and CD8 promoter. According to another embodiment, the cell-specific promoter is CD3 promoter. According to some embodiments, the CD3 promoter has nucleic acid sequence SEQ ID NO: 17.

The term “pharmaceutical composition” as used herein refers to a composition comprising at least one pseudotyped virus as disclosed herein formulated together with one or more pharmaceutically acceptable carriers and optionally excipients.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules. According to some embodiments, the pharmaceutical composition is a solution of injection.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffins.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerine and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject. The carrier may comprise, in total, from about 0.1% to about 99.99999% by weight of the pharmaceutical compositions presented herein.

According to some embodiments, the pharmaceutical composition of the present invention is administered systemically. According to some embodiments, the composition is administered parenterally. According to other embodiments, the pharmaceutical composition of the present invention is administered locally, e.g., intratumorraly. In some embodiments, the pharmaceutical composition is formulated for systemic administration. In some embodiments, the pharmaceutical composition is formulated for intratumoral administration. In some embodiments, the pharmaceutical composition is formulated for administration to a subject.

The pharmaceutical composition of the present invention may be administered by any known of administration. The term “administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administrations, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. according to some embodiments, the composition is parenterally administered.

The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

According to some embodiments, the pharmaceutical composition of the present invention is for use in treating cancer.

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

The term “cancer” comprises cancerous diseases or a tumor being treated or prevented that is selected from the group comprising, but not limited to, mammary carcinomas, melanoma, skin neoplasms, lymphoma, leukemia, gastrointestinal tumors, including colon carcinomas, stomach carcinomas, pancreas carcinomas, colon cancer, and small intestine cancer, ovarian carcinomas, cervical carcinomas, lung cancer, prostate cancer, kidney cell carcinomas and/or liver metastases.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer expresses an antigen targeted by the molecule of interest. In some embodiments, the cancer expresses an antigen bound by the molecule of interest. In some embodiments, the cancer expresses an antigen targeted by the CAR. n some embodiments, the cancer expresses an antigen bound by the CAR.

According to some embodiments, the pharmaceutical composition of the present inventor is co-administered with another treatment of cancer.

According to some embodiments, upon use, T-cells of the subject express the CAR encoded by the pseudotyped virus.

According to another aspect, the present invention provides a method of treating cancer in a subject in need thereof comprising administering the pseudotyped virus of the present invention, or the pharmaceutical composition of the current invention to the subject, thereby treating cancer.

According to some embodiments, the method comprises systemically or intratumorally administering the virus or composition. In some embodiments, the method comprises systemic administration. In some embodiments, the method comprises intratumoral administration. According to some embodiments, administering the pseudotyped virus comprises infecting T-cells and/or expressing the receptor in T-cells.

According to one embodiment, the cancer is as described in any one of the above embodiments. According to some embodiments, the cancer is selected from leukemia, lymphoma, melanoma, neuroendocrine tumor, carcinoma and sarcoma. In some embodiments, the cancer is a solid cancer. In some embodiments, the cancer is a tumor. In some embodiments, the cancer is a hematopoietic cancer. In some embodiments, treating comprises infecting T cells with the pseudotyped virus.

In some embodiments, the method comprises administering an effective amount of the virus or composition. In some embodiments, an effective amount is a therapeutically effective amount. In some embodiments, an effective amount is an amount sufficient to treat at least one symptom. In some embodiments, an effective amount is an amount sufficient to retard tumor growth. In some embodiments, an effective amount is an amount sufficient to shrink a tumor.

In some embodiments, the method further comprises providing a sample comprising T cells. In some embodiments, the sample is a blood sample. In some embodiments, the blood is peripheral blood. In some embodiments, T cells are isolated form the sample. In some embodiments, T cells are not isolated. In some embodiments, the T cells are contacted with the pseudotyped virus or the composition of the invention. In some embodiments, the sample is contacted. In some embodiments, the contacting is under conditions sufficient for infection of the T cells. In some embodiments, infection is infection by the pseudotyped virus. In some embodiments, the conditions are in culture. In some embodiments, the method comprises administering the infected T cells to the subject.

In some embodiments, the method is an in vivo method. In some embodiments, the method is an ex vivo method. In some embodiments, the method is an in vitro method. In some embodiments, the composition is for use in a method of the invention. In some embodiments, the pseudotyped virus is for use in a method of the invention. In some embodiments, the fusion protein is for use in a method of the invention.

According to another aspect, the present invention provides a method for in situ transducing T-cell with a nucleic acid encoding for to a receptor selected from a chimeric antigen receptor (CAR) and T-cell receptor, wherein the method comprises administering to a subject the pseudotyped virus of the present invention.

By another aspect, there is provided a method for expressing a molecule of interest in a T cell, the method comprising contacting the T cell with the pseudotyped virus of the invention or composition of the invention, thereby expressing a molecule of interest in a T cell.

In some embodiments, the contacting is performed in vitro. In some embodiments, the contacting is performed in situ. In some embodiments, the contacting is performed in a subject. In some embodiments, the subject is a subject in need thereof.

According to some embodiments, the pseudotyped virus comprises a nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3). According to some other embodiments, the pseudotyped virus comprises a nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3), and wherein the nucleic acid encoding for a receptor is operably linked to a cell-specific promoter. According to some embodiments, the pseudotyped virus comprises a nucleic acid encoding for a receptor selected from a chimeric antigen receptor (CAR) and T-cell receptor, wherein the nucleic acid encoding for the receptor is operably linked to a cell-specific promoter. According to one embodiment, the cell-specific promoter is selected from CD3, CD4 and CD8 promoter. According to another embodiment, the cell-specific promoter is CD3 promoter. According to some embodiments, the CD3 promoter has nucleic acid sequence SEQ ID NO: 17. According to some embodiments, the fusion protein comprises amino acid sequence SEQ ID NO: 11. According to other embodiments, the fusion protein comprises amino acid sequence SEQ ID NO: 12. According to other embodiments, the fusion protein comprises amino acid sequence selected from SEQ ID NOs: 39-51. According to some embodiments, the fusion protein comprises amino acid sequence SEQ ID NO: 64. According to other embodiments, the fusion protein comprises amino acid sequence SEQ ID NO: 65. According to other embodiments, the fusion protein comprises amino acid sequence selected from SEQ ID NOs: 66-78.

According to another aspect, the present invention provides a method for expressing a chimeric antigen receptor in T-cells comprising contacting T-cells with the pseudotyped virus of the present invention, thereby transducing the T-cells with a nucleic acid encoding the CAR. According to some embodiments, the expression is performed in situ and contacting T-cell with pseudotyped virus comprises administering the pseudotyped virus to a subject.

According to some embodiments, the present invention provides a population of T-cells expressing a chimeric antigen receptor or a T-cell receptor, the T-cells are obtained by contacting or infecting the T-cells with the pseudotyped virus of the present invention.

According to some embodiments, the pseudotyped virus comprises an nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3). According to some other embodiments, the pseudotyped virus comprises a nucleic acid encoding for a receptor selected from a chimeric antigen receptor (CAR) and T-cell receptor, wherein the pseudotyped virus comprises a fusion protein comprising a vesicular stomatitis virus envelope glycoprotein (VSVG) or a fragment or an analog thereof linked to a polypeptide comprising an antigen binding domain specifically binding to a cluster of differentiation 3 (CD3), and wherein the nucleic acid encoding for a receptor is operably linked to a cell-specific promoter. According to some embodiments, the pseudotyped virus comprises a nucleic acid encoding for a receptor selected from a chimeric antigen receptor (CAR) and T-cell receptor, wherein the nucleic acid encoding for the receptor is operably linked to a cell-specific promoter. According to one embodiment, the cell-specific promoter is selected from CD3, CD4 and CD8 promoter. According to another embodiment, the cell-specific promoter is CD3 promoter. According to some embodiments, the CD3 promoter has nucleic acid sequence SEQ ID NO: 17. According to some embodiments, the fusion protein comprises amino acid sequence SEQ ID NO: 11. According to other embodiments, the fusion protein comprises amino acid sequence SEQ ID NO: 12. According to other embodiments, the fusion protein comprises amino acid sequence selected from SEQ ID NOs: 39-51. According to some embodiments, the fusion protein comprises amino acid sequence SEQ ID NO: 64. According to other embodiments, the fusion protein comprises amino acid sequence SEQ ID NO: 65. According to other embodiments, the fusion protein comprises amino acid sequence selected from SEQ ID NOs: 66-78.

According to any one of the above embodiments, all definitions and embodiments related to pseudotyped virus, chimeric antigen receptor (CAR), T-cell receptor, nucleic acid, fusion protein linker, promoter etc. apply herein as well as form separate embodiments.

By another aspect, there is provided a T cell produced by a method of the invention.

By another aspect, there is provided a pharmaceutical composition comprises a T cell produced by a method of the invention.

The terms “comprising”, “comprise(s)”, “include(s)”, “having”, “has” and “contain(s),” are used herein interchangeably and have the meaning of “consisting at least in part of′. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Sequence Table

| SEQ | | |
|---|---|---|
| ID | Sequence | |
| No: | name | Sequence |
| 1 | VSVG | MKCLLYLAFLFIGVNCKFTIVFPHNQKGNWKNVPSNYHYCPSSS |
| | (prot) | DLNWHNDLIGTALQVKMPKSHKAIQADGWMCHASKWVTTCDF |
| | | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 2 | VSVG | atgaagtgccttttgtacttagcctttttattcattggggtgaattgcaagttcaccatagtttttccacacaac |
| | (DNA) | caaaaaggaaactggaaaaatgttccttctaattaccattattgcccgtcaagctcagatttaaattggcat |
| | | aatgacttaataggcacagccttacaagtcaaaatgcccaagagtcacaaggctattcaagcagacggt |
| | | tggatgtgtcatgcttccaaatgggtcactacttgtgatttccgctggtatggaccgaagtatataacacat |
| | | tccatccgatccttcactccatctgtagaacaatgcaaggaaagcattgaacaaacgaaacaaggaact |
| | | tggctgaatccaggcttccctcctcaaagttgtggatatgcaactgtgacggatgccgaagcagtgattg |
| | | tccaggtgactcctcaccatgtgctggttgatgaatacacaggagaatgggttgattcacagttcatcaa |
| | | cggaaaatgcagcaattacatatgccccactgtccataactctacaacctggcattctgactataaggtc |
| | | aaagggctatgtgattctaacctcatttccatggacatcaccttcttctcagaggacggagagctatcatc |
| | | cctgggaaaggagggcacagggttcagaagtaactactttgcttatgaaactggaggcaaggcctgca |
| | | aaatgcaatactgcaagcattggggagtcagactcccatcaggtgtctggttcgagatggctgataagg |
| | | atctctttgctgcagccagattccctgaatgcccagaagggtcaagtatctctgctccatctcagacctca |
| | | gtggatgtaagtctaattcaggacgttgagaggatcttggattattccctctgccaagaaacctggagca |
| | | aaatcagagcgggtcttccaatctctccagtggatctcagctatcttgctcctaaaaacccaggaaccgg |
| | | tcctgctttcaccataatcaatggtaccctaaaatactttgagaccagatacatcagagtcgatattgctgc |
| | | tccaatcctctcaagaatggtcggaatgatcagtggaactaccacagaaagggaactgtgggatgact |
| | | gggcaccatatgaagacgtggaaattggacccaatggagttctgaggaccagttcaggatataagtttc |
| | | ctttatacatgattggacatggtatgttggactccgatcttcatcttagctcaaaggctcaggtgttcgaac |
| | | atcctcacattcaagacgctgcttcgcaacttcctgatgatgagagtttattttttggtgatactgggctatc |
| | | caaaaatccaatcgagcttgtagaaggttggttcagtagttggaaaagctctattgcctcttttttctttatca |
| | | tagggttaatcattggactattcttggttctccgagttggtatccatctttgcattaaattaaagcacaccaa |
| | | gaaaagacagatttatacagacatagagatgaaccgacttggaaagtaa |
| 3 | OKT3 | MEAPAQLLFLLLLWLPDTTGQIVLTQSPAIMSASPGEKVTMTCSA |
| | VL-VH | SSSVSYMNWYQQKSGTSPKRWIYDTSKLASGVPAHFRGSGSGTS |
| | GGGGS | YSLTISGMEAEDAATYYCQQWSSNPFTFGSGTKLEINGGGGSGG |
| | x4 | GGSGGGGSGGGGSQVQLQQSGAELARPGASVKMSCKASGYTF |
| | (prot) | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSS |
| 4 | OKT3 | ATGGAGGCACCGGCGCAGCTTCTGTTTCTGCTGCTTCTCTGGC |
| | VL-VH | TGCCGGACACAACAGGCCAAATCGTGCTCACACAGTCCCCTG |
| | GGGGS | CAATCATGAGTGCCTCTCCGGGCGAGAAGGTAACCATGACCT |
| | x4 | GCAGTGCTTCATCTTCAGTTTCATATATGAACTGGTATCAACA |
| | (DNA) | GAAGTCTGGTACGTCACCAAAGCGGTGGATTTATGACACCTC |
| | | CAAATTGGCTAGCGGTGTGCCTGCGCATTTTCGGGGGAGTGG |
| | | ATCTGGGACATCATATAGTCTGACTATAAGCGGAATGGAAGC |
| | | TGAGGACGCGGCAACTTACTATTGCCAACAATGGAGTAGTAA |
| | | CCCATTCACTTTCGGGAGCGGCACTAAGCTGGAGATCAACGG |
| | | CGGGGGAGGGTCCGGAGGTGGTGGTAGTGGTGGCGGGGGAT |
| | | CCGGTGGAGGGGGCAGTCAAGTGCAATTGCAGCAATCAGGAG |
| | | CAGAACTTGCACGGCCCGGTGCTTCCGTGAAAATGAGCTGTA |
| | | AAGCTTCAGGCTACACTTTTACTCGATACACGATGCACTGGGT |
| | | GAAACAGCGGCCAGGGCAGGGCCTGGAGTGGATTGGGTATAT |
| | | TAACCCGTCACGAGGATACACAAATTACAATCAGAAATTTAA |
| | | AGACAAAGCCACGCTTACAACTGATAAGAGTTCTTCCACGGC |
| | | TTATATGCAACTGTCATCCTTGACTTCCGAGGATTCAGCAGTG |
| | | TATTATTGTGCGCGGTACTATGATGACCACTATTGCCTTGATT |
| | | ATTGGGGACAGGGAACGACACTTACCGTAAGCTCT |
| 5 | CD8a | KPTTTPAPRPPTPAPTIASQPLSLRPEACRPAAGGAVHTRGL |
| | (prot) | |
| 6 | CD8a | aagcccaccaccacccctgcccctagacctccaaccccagcccctacaatcgccagccagcccctga |
| | (DNA) | gcctgaggcccgaagcctgtagacctgccgctggcggagccgtgcacaccagaggcctg |
| 7 | IgD | SPKAQASSVPTAQPQAEGSLAKATTAPATTRNTGRGGEEKKKEK |
| | Hinge | EKEEQEERETKTPE |
| | (prot) | |
| 8 | IgD | TCTCCAAAGGCACAGGCCTCCTCAGTGCCCACTGCACAACCC |
| | Hinge | CAAGCAGAGGGCAGCCTCGCCAAGGCAACCACAGCCCCAGCC |
| | (DNA) | ACCACCCGTAACACAGGAAGAGGAGGAGAAGAGAAGAAGAA |
| | | GGAGAAGGAGAAAGAGGAACAAGAAGAGAGAGAGACAAAG |
| | | ACACCAGAG |
| 9 | IgGI | DKTHTCPPCPAPELLGG |
| | hinge | |
| | (prot) | |
| 10 | IgGI | GACAAAACTCACACATGCCCACCGTGCCCAGCACCTGAACTC |
| | hinge | CTGGGGGGA |
| | (DNA) | |
| 11 | OKT3 | MEAPAQLLFLLLLWLPDTTGQIVLTQSPAIMSASPGEKVTMTCSA |
| | (VL-VH) | SSSVSYMNWYQQKSGTSPKRWIYDTSKLASGVPAHFRGSGSGTS |
| | VSVG- | YSLTISGMEAEDAATYYCQQWSSNPFTFGSGTKLEINGGGGSGG |
| | | GGSGGGGSGGGGSQVQLQQSGAELARPGASVKMSCKASGYTF |
| | | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSMKCLLYLAFLFIGVNCKFTIVFPHNQKGNWKNVPSNYHYC |
| | | PSSSDLNWHNDLIGTALQVKMPKSHKAIQADGWMCHASKWVT |
| | | TCDFRWYGPKYITHSIRSFTPSVEQCKESIEQTKQGTWLNPGFPP |
| | | QSCGYATVTDAEAVIVQVTPHHVLVDEYTGEWVDSQFINGKCS |
| | | NYICPTVHNSTTWHSDYKVKGLCDSNLISMDITFFSEDGELSSLG |
| | | KEGTGFRSNYFAYETGGKACKMQYCKHWGVRLPSGVWFEMAD |
| | | KDLFAAARFPECPEGSSISAPSQTSVDVSLIQDVERILDYSLCQET |
| | | WSKIRAGLPISPVDLSYLAPKNPGTGPAFTIINGTLKYFETRYIRV |
| | | DIAAPILSRMVGMISGTTTERELWDDWAPYEDVEIGPNGVLRTSS |
| | | GYKFPLYMIGHGMLDSDLHLSSKAQVFEHPHIQDAASQLPDDES |
| | | LFFGDTGLSKNPIELVEGWFSSWKSSIASFFFIIGLIIGLFLVLRVGI |
| | | HLCIKLKHTKKRQIYTDIEMNRLGK |
| 12 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | CD8a- | GGSGGGGSGGGGSQVQLQQSGAELARPGASVKMSCKASGYTF |
| | VSVG | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSKPTTTPAPRPPTPAPTIASQPLSLRPEACRPAAGGAVHT |
| | | RGLMKCLLYLAFLFIGVNCKFTIVFPHNQKGNWKNVPSNYHYC |
| | | PSSSDLNWHNDLIGTALQVKMPKSHKAIQADGWMCHASKWVT |
| | | TCDFRWYGPKYITHSIRSFTPSVEQCKESIEQTKQGTWLNPGFPP |
| | | QSCGYATVTDAEAVIVQVTPHHVLVDEYTGEWVDSQFINGKCS |
| | | NYICPTVHNSTTWHSDYKVKGLCDSNLISMDITFFSEDGELSSLG |
| | | KEGTGFRSNYFAYETGGKACKMQYCKHWGVRLPSGVWFEMAD |
| | | KDLFAAARFPECPEGSSISAPSQTSVDVSLIQDVERILDYSLCQET |
| | | WSKIRAGLPISPVDLSYLAPKNPGTGPAFTIINGTLKYFETRYIRV |
| | | DIAAPILSRMVGMISGTTTERELWDDWAPYEDVEIGPNGVLRTSS |
| | | GYKFPLYMIGHGMLDSDLHLSSKAQVFEHPHIQDAASQLPDDES |
| | | LFFGDTGLSKNPIELVEGWFSSWKSSIASFFFIIGLIIGLFLVLRVGI |
| | | HLCIKLKHTKKRQIYTDIEMNRLGK |
| 13 | 2C11 | MEAPAQLLFLLLLWLPDTTGDIQMTQSPSSLPASLGDRVTINCQA |
| | (prot) | SQDISNYLNWYQQKPGKAPKLLIYYTNKLADGVPSRFSGSGSGR |
| | | DSSFTISSLESEDIGSYYCQQYYNYPWTFGPGTKLEIKGGGGSGG |
| | | GGSGGGGSGGGGSEVQLVESGGGLVQPGKSLKLSCEASGFTFSG |
| | | YGMHWVRQAPGRGLESVAYITSSSINIKYADAVKGRFTVSRDNA |
| | | KNLLFLQMNILKSEDTAMYYCARFDWDKNYWGQGTMVTVSS |
| 14 | 2C11 | atggaggcacctgcacaacttctgttcttgctcttgctttggctccccgacacaacaggtgacatacagat |
| | (DNA) | gacacagagtccttctagtctcccagccagcctcggagacagggttacgataaattgtcaggccagtca |
| | | ggacatatctaactatcttaattggtaccagcagaaaccgggtaaggcaccgaagttgctgatctattac |
| | | actaacaagttggcggacggtgtgccatctagattctccggctcaggtagtgggcgcgacagcagtttt |
| | | actatctctagccttgaatctgaagatattgggtcatactactgccaacaatactacaactacccgtggac |
| | | gttcggacctgggacaaagcttgagataaagggagggggaggaagtggcggcgggggcagtggtg |
| | | gaggaggatcaggcggtgggggttctgaagtacaattggttgaatctggtggaggattggtgcaaccg |
| | | ggcaaaagcttgaaactgtcctgcgaagctagtggttttaccttttctgggtacggaatgcattgggttcg |
| | | acaagctccaggaagggggctcgaatctgttgcttatataacaagctcaagcatcaacattaaatatgca |
| | | gacgcggtaaaaggacgatttacggtcagtagggataacgctaaaaacctgctgttcctccagatgaat |
| | | attctcaagtcagaggatactgctatgtattattgcgctcggtttgattgggataaaaattactggggacaa |
| | | ggaaccatggtaaccgtctcctcc |
| 15 | 2C11- | MEAPAQLLFLLLLWLPDTTGDIQMTQSPSSLPASLGDRVTINCQA |
| | VSVG | SQDISNYLNWYQQKPGKAPKLLIYYTNKLADGVPSRFSGSGSGR |
| | | DSSFTISSLESEDIGSYYCQQYYNYPWTFGPGTKLEIKGGGGSGG |
| | | GGSGGGGSGGGGSEVQLVESGGGLVQPGKSLKLSCEASGFTFS |
| | | GYGMHWVRQAPGRGLESVAYITSSSINIKYADAVKGRFTVSRDN |
| | | AKNLLFLQMNILKSEDTAMYYCARFDWDKNYWGQGTMVTVSS |
| | | MKCLLYLAFLFIGVNCKFTIVFPHNQKGNWKNVPSNYHYCPSSS |
| | | DLNWHNDLIGTALQVKMPKSHKAIQADGWMCHASKWVTTCDF |
| | | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 16 | 2C11- | MEAPAQLLFLLLLWLPDTTGDIQMTQSPSSLPASLGDRVTINCQA |
| | CD8a- | SQDISNYLNWYQQKPGKAPKLLIYYTNKLADGVPSRFSGSGSGR |
| | VSVG | DSSFTISSLESEDIGSYYCQQYYNYPWTFGPGTKLEIKGGGGSGG |
| | | GGSGGGGSGGGGSEVQLVESGGGLVQPGKSLKLSCEASGFTFSG |
| | | YGMHWVRQAPGRGLESVAYITSSSINIKYADAVKGRFTVSRDNA |
| | | KNLLFLQMNILKSEDTAMYYCARFDWDKNYWGQGTMVTVSSK |
| | | PTTTPAPRPPTPAPTIASQPLSLRPEACRPAAGGAVHTRGLMK |
| | | CLLYLAFLFIGVNCKFTIVFPHNQKGNWKNVPSNYHYCPSSSDL |
| | | NWHNDLIGTALQVKMPKSHKAIQADGWMCHASKWVTTCDFR |
| | | WYGPKYITHSIRSFTPSVEQCKESIEQTKQGTWLNPGFPPQSCGY |
| | | ATVTDAEAVIVQVTPHHVLVDEYTGEWVDSQFINGKCSNYICPT |
| | | VHNSTTWHSDYKVKGLCDSNLISMDITFFSEDGELSSLGKEGTGF |
| | | RSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 17 | CD3 | ctgatcagaaacaagaggatctgctaatgtggcttgtgtaatagtttctctcagtggatctaaagaggtgg |
| | promoter | cacttccttgtttgtgagtaaatgtagttgtattgttcataggaagcccaaaatcttctttgtttgttaccagtg |
| | | actattgctatttgttttttgttaccaggaaaggctattctaaattcccttacacttcctagaattctgaacaaa |
| | | ctttcctcatagtctctgctttgtatcaatcatcccaaaatctttgcttcagaaagagcagtttgcacctgtttg |
| | | tgttttaccccacacctagcccactgttgctttgacagtcttacaccatcaaaggcagaaggtagagagg |
| | | cagatttcttctagttcctcccccactcttcctttttctgaatacccatgagtctgttggcggaggatagtgct |
| 18 | Truncated | EHPHIQDAASQLPDDESLFFGDTGLSKNPIELVEGWFSSWKSSIAS |
| | VSVG | FFFIIGLIIGLFLVLRVGIHLCIKLKHTKKRQIYTDIEMNRLGK |
| | (prot) | |
| 19 | Truncated | cgaacatcctcacattcaagacgctgcttcgcaacttcctgatgatgagagtttattttttggtgatactgg |
| | VSVG | gctatccaaaaatccaatcgagcttgtagaaggttggttcagtagttggaaaagctctattgcctcttttttc |
| | (DNA) | tttatcatagggttaatcattggactattcttggttctccgagttggtatccatctttgcattaaattaaagcac |
| | | accaagaaaagacagatttatacagacatagagatgaaccgacttggaaagtaa |
| 20 | IgG2a | RGPTIKPCPPCKCPAPNLLG |
| | Linker | |
| | Prot | |
| 21 | IgG2a | agagggcccacaatcaagccctgtcctccatgcaaatgcccagcacctaacctcttgggt |
| | Linker | |
| | DNA | |
| 22 | IgD | QASSVPTAQPQAEGSLAKATTAPATTRNTGRGGEEKKKEKEK |
| | Linker | |
| | (short) | |
| | Prot | |
| 23 | IgD | caggcctcctcagtgcccactgcacaaccccaagcagagggcagcctcgccaaggcaaccacagc |
| | Linker | cccagccaccacccgtaacacaggaagaggaggagaagagaagaagaaggagaaggagaaa |
| | (short) | |
| | DNA | |
| 24 | IgD | RWPESPKAQASSVPTAQPQAEGSLAKATTAPATTRNTGRGGEEK |
| | linker | KKEKEKEEQEERETKTPECP |
| | (Long | |
| | #2) prot | |
| 25 | IgD | cgctggccagagtctccaaaggcacaggcctcctcagtgcccactgcacaaccccaagcagagggc |
| | linker | agcctcgccaaggcaaccacagccccagccaccacccgtaacacaggaagaggaggagaagaga |
| | (Long | agaagaaggagaaggagaaagaggaacaagaagagagagagacaaagacaccagagtgtccg |
| | #2) DNA | |
| 26 | Linker | GGGGS |
| 27 | Linker | GGGGSGGGGS |
| 28 | Linker | GGGGSGGGGSGGGGS |
| 29 | Linker | GGGGSGGGGSGGGGSGGGGS |
| 30 | Linker | Ggagggggaggaagtggcggcgggggcagt |
| | DNA | |
| 31 | Linker | Ggagggggaggaagtggcggcgggggcagtggtggaggaggatca |
| | DNA | |
| 32 | Linker | ggagggggaggaagtggcggcgggggcagtggtggaggaggatcaggcggtgggggttct |
| | DNA | |
| 33 | VSVG | MKCLLYLAFLFIGVNCKFTIVFPHNQKGNWKNVPSNYHYCPSSS |
| | (K47Q) | DLNWHNDLIGTALQVKMPQSHKAIQADGWMCHASKWVTTCDF |
| | prot | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 34 | VSVG | atgaagtgccttttgtacttagcctttttattcattggggtgaattgcaagttcaccatagtttttccacacaac |
| | (K47Q) | caaaaaggaaactggaaaaatgttccttctaattaccattattgcccgtcaagctcagatttaaattggcat |
| | DNA | aatgacttaataggcacagccttacaagtcaaaatgccccagagtcacaaggctattcaagcagacggt |
| | | tggatgtgtcatgcttccaaatgggtcactacttgtgatttccgctggtatggaccgaagtatataacacat |
| | | tccatccgatccttcactccatctgtagaacaatgcaaggaaagcattgaacaaacgaaacaaggaact |
| | | tggctgaatccaggcttccctcctcaaagttgtggatatgcaactgtgacggatgccgaagcagtgattg |
| | | tccaggtgactcctcaccatgtgctggttgatgaatacacaggagaatgggttgattcacagttcatcaa |
| | | cggaaaatgcagcaattacatatgccccactgtccataactctacaacctggcattctgactataaggtc |
| | | aaagggctatgtgattctaacctcatttccatggacatcaccttcttctcagaggacggagagctatcatc |
| | | cctgggaaaggagggcacagggttcagaagtaactactttgcttatgaaactggaggcaaggcctgca |
| | | aaatgcaatactgcaagcattggggagtcagactcccatcaggtgtctggttcgagatggctgataagg |
| | | atctctttgctgcagccagattccctgaatgcccagaagggtcaagtatctctgctccatctcagacctca |
| | | gtggatgtaagtctaattcaggacgttgagaggatcttggattattccctctgccaagaaacctggagca |
| | | aaatcagagcgggtcttccaatctctccagtggatctcagctatcttgctcctaaaaacccaggaaccgg |
| | | tcctgctttcaccataatcaatggtaccctaaaatactttgagaccagatacatcagagtcgatattgctgc |
| | | tccaatcctctcaagaatggtcggaatgatcagtggaactaccacagaaagggaactgtgggatgact |
| | | gggcaccatatgaagacgtggaaattggacccaatggagttctgaggaccagttcaggatataagtttc |
| | | ctttatacatgattggacatggtatgttggactccgatcttcatcttagctcaaaggctcaggtgttcgaac |
| | | atcctcacattcaagacgctgcttcgcaacttcctgatgatgagagtttattttttggtgatactgggctatc |
| | | caaaaatccaatcgagcttgtagaaggttggttcagtagttggaaaagctctattgcctcttttttctttatca |
| | | tagggttaatcattggactattcttggttctccgagttggtatccatctttgcattaaattaaagcacaccaa |
| | | gaaaagacagatttatacagacatagagatgaaccgacttggaaagtaa |
| 35 | VSVG | MKCLLYLAFLFIGVNCKFTIVFPHNQKGNWKNVPSNYHYCPSSS |
| | (R354A) | DLNWHNDLIGTALQVKMPKSHKAIQADGWMCHASKWVTTCDF |
| | prot | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTEAELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 36 | VSVG | atgaagtgccttttgtacttagcctttttattcattggggtgaattgcaagttcaccatagtttttccacacaac |
| | (R354A) | caaaaaggaaactggaaaaatgttccttctaattaccattattgcccgtcaagctcagatttaaattggcat |
| | DNA | aatgacttaataggcacagccttacaagtcaaaatgcccaagagtcacaaggctattcaagcagacggt |
| | | tggatgtgtcatgcttccaaatgggtcactacttgtgatttccgctggtatggaccgaagtatataacacat |
| | | tccatccgatccttcactccatctgtagaacaatgcaaggaaagcattgaacaaacgaaacaaggaact |
| | | tggctgaatccaggcttccctcctcaaagttgtggatatgcaactgtgacggatgccgaagcagtgattg |
| | | tccaggtgactcctcaccatgtgctggttgatgaatacacaggagaatgggttgattcacagttcatcaa |
| | | cggaaaatgcagcaattacatatgccccactgtccataactctacaacctggcattctgactataaggtc |
| | | aaagggctatgtgattctaacctcatttccatggacatcaccttcttctcagaggacggagagctatcatc |
| | | cctgggaaaggagggcacagggttcagaagtaactactttgcttatgaaactggaggcaaggcctgca |
| | | aaatgcaatactgcaagcattggggagtcagactcccatcaggtgtctggttcgagatggctgataagg |
| | | atctctttgctgcagccagattccctgaatgcccagaagggtcaagtatctctgctccatctcagacctca |
| | | gtggatgtaagtctaattcaggacgttgagaggatcttggattattccctctgccaagaaacctggagca |
| | | aaatcagagcgggtcttccaatctctccagtggatctcagctatcttgctcctaaaaacccaggaaccgg |
| | | tcctgctttcaccataatcaatggtaccctaaaatactttgagaccagatacatcagagtcgatattgctgc |
| | | tccaatcctctcaagaatggtcggaatgatcagtggaactaccacagaagcggaactgtgggatgact |
| | | gggcaccatatgaagacgtggaaattggacccaatggagttctgaggaccagttcaggatataagtttc |
| | | ctttatacatgattggacatggtatgttggactccgatcttcatcttagctcaaaggctcaggtgttcgaac |
| | | atcctcacattcaagacgctgcttcgcaacttcctgatgatgagagtttattttttggtgatactgggctatc |
| | | caaaaatccaatcgagcttgtagaaggttggttcagtagttggaaaagctctattgcctcttttttctttatca |
| | | tagggttaatcattggactattcttggttctccgagttggtatccatctttgcattaaattaaagcacaccaa |
| | | gaaaagacagatttatacagacatagagatgaaccgacttggaaagtaa |
| 37 | VSVG | MKCLLYLAFLFIGVNCKFTIVFPHNQKGNWKNVPSNYHYCPSSS |
| | (K47Q/ | DLNWHNDLIGTALQVKMPQSHKAIQADGWMCHASKWVTTCDF |
| | R354A | RWYGPKYITHSIRSFTPSVEQCKESIEQTKQGTWLNPGFPPQSCG |
| | prot | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTEAELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 38 | VSVG | atgaagtgccttttgtacttagcctttttattcattggggtgaattgcaagttcaccatagtttttccacacaac |
| | (K47Q/ | caaaaaggaaactggaaaaatgttccttctaattaccattattgcccgtcaagctcagatttaaattggcat |
| | R354A | aatgacttaataggcacagccttacaagtcaaaatgccccagagtcacaaggctattcaagcagacggt |
| | DNA | tggatgtgtcatgcttccaaatgggtcactacttgtgatttccgctggtatggaccgaagtatataacacat |
| | | tccatccgatccttcactccatctgtagaacaatgcaaggaaagcattgaacaaacgaaacaaggaact |
| | | tggctgaatccaggcttccctcctcaaagttgtggatatgcaactgtgacggatgccgaagcagtgattg |
| | | tccaggtgactcctcaccatgtgctggttgatgaatacacaggagaatgggttgattcacagttcatcaa |
| | | cggaaaatgcagcaattacatatgccccactgtccataactctacaacctggcattctgactataaggtc |
| | | aaagggctatgtgattctaacctcatttccatggacatcaccttcttctcagaggacggagagctatcatc |
| | | cctgggaaaggagggcacagggttcagaagtaactactttgcttatgaaactggaggcaaggcctgca |
| | | aaatgcaatactgcaagcattggggagtcagactcccatcaggtgtctggttcgagatggctgataagg |
| | | atctctttgctgcagccagattccctgaatgcccagaagggtcaagtatctctgctccatctcagacctca |
| | | gtggatgtaagtctaattcaggacgttgagaggatcttggattattccctctgccaagaaacctggagca |
| | | aaatcagagcgggtcttccaatctctccagtggatctcagctatcttgctcctaaaaacccaggaaccgg |
| | | tcctgctttcaccataatcaatggtaccctaaaatactttgagaccagatacatcagagtcgatattgctgc |
| | | tccaatcctctcaagaatggtcggaatgatcagtggaactaccacagaagcggaactgtgggatgact |
| | | gggcaccatatgaagacgtggaaattggacccaatggagttctgaggaccagttcaggatataagtttc |
| | | ctttatacatgattggacatggtatgttggactccgatcttcatcttagctcaaaggctcaggtgttcgaac |
| | | atcctcacattcaagacgctgcttcgcaacttcctgatgatgagagtttattttttggtgatactgggctatc |
| | | caaaaatccaatcgagcttgtagaaggttggttcagtagttggaaaagctctattgcctcttttttctttatca |
| | | tagggttaatcattggactattcttggttctccgagttggtatccatctttgcattaaattaaagcacaccaa |
| | | gaaaagacagatttatacagacatagagatgaaccgacttggaaagtaa |
| 39 | IgD | MEAPAQLLFLLLLWLPDTTGQIVLTQSPAIMSASPGEKVTMTCSA |
| | Hinge- | SSSVSYMNWYQQKSGTSPKRWIYDTSKLASGVPAHFRGSGSGTS |
| | VSVG | YSLTISGMEAEDAATYYCQQWSSNPFTFGSGTKLEINGGGGSGG |
| | | GGSGGGGSGGGGSQVQLQQSGAELARPGASVKMSCKASGYTF |
| | | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSSPKAQASSVPTAQPQAEGSLAKATTAPATTRNTGRGGE |
| | | EKKKEKEKEEQEERETKTPEMKCLLYLAFLFIGVNCKFTIVFP |
| | | HNQKGNWKNVPSNYHYCPSSSDLNWHNDLIGTALQVKMPKSH |
| | | KAIQADGWMCHASKWVTTCDFRWYGPKYITHSIRSFTPSVEQC |
| | | KESIEQTKQGTWLNPGFPPQSCGYATVTDAEAVIVQVTPHHVLV |
| | | DEYTGEWVDSQFINGKCSNYICPTVHNSTTWHSDYKVKGLCDS |
| | | NLISMDITFFSEDGELSSLGKEGTGFRSNYFAYETGGKACKMQY |
| | | CKHWGVRLPSGVWFEMADKDLFAAARFPECPEGSSISAPSQTSV |
| | | DVSLIQDVERILDYSLCQETWSKIRAGLPISPVDLSYLAPKNPGTG |
| | | PAFTIINGTLKYFETRYIRVDIAAPILSRMVGMISGTTTERELWDD |
| | | WAPYEDVEIGPNGVLRTSSGYKFPLYMIGHGMLDSDLHLSSKAQ |
| | | VFEHPHIQDAASQLPDDESLFFGDTGLSKNPIELVEGWFSSWKSSI |
| | | ASFFFIIGLIIGLFLVLRVGIHLCIKLKHTKKRQIYTDIEMNRLGK |
| 40 | OKT3- | MEAPAQLLFLLLLWLPDTTGQIVLTQSPAIMSASPGEKVTMTCSA |
| | IgG1 | SSSVSYMNWYQQKSGTSPKRWIYDTSKLASGVPAHFRGSGSGTS |
| | hinge- | YSLTISGMEAEDAATYYCQQWSSNPFTFGSGTKLEINGGGGSGG |
| | VSVG | GGSGGGGSGGGGSQVQLQQSGAELARPGASVKMSCKASGYTF |
| | | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSDKTHTCPPCPAPELLGGMKCLLYLAFLFIGVNCKFTIVFP |
| | | HNQKGNWKNVPSNYHYCPSSSDLNWHNDLIGTALQVKMPKSH |
| | | KAIQADGWMCHASKWVTTCDFRWYGPKYITHSIRSFTPSVEQC |
| | | KESIEQTKQGTWLNPGFPPQSCGYATVTDAEAVIVQVTPHHVLV |
| | | DEYTGEWVDSQFINGKCSNYICPTVHNSTTWHSDYKVKGLCDS |
| | | NLISMDITFFSEDGELSSLGKEGTGFRSNYFAYETGGKACKMQY |
| | | CKHWGVRLPSGVWFEMADKDLFAAARFPECPEGSSISAPSQTSV |
| | | DVSLIQDVERILDYSLCQETWSKIRAGLPISPVDLSYLAPKNPGTG |
| | | PAFTIINGTLKYFETRYIRVDIAAPILSRMVGMISGTTTERELWDD |
| | | WAPYEDVEIGPNGVLRTSSGYKFPLYMIGHGMLDSDLHLSSKAQ |
| | | VFEHPHIQDAASQLPDDESLFFGDTGLSKNPIELVEGWFSSWKSSI |
| | | ASFFFIIGLIIGLFLVLRVGIHLCIKLKHTKKRQIYTDIEMNRLGK |
| 41 | OKT3 | MEAPAQLLFLLLLWLPDTTGQIVLTQSPAIMSASPGEKVTMTCSA |
| | (VL-VH) | SSSVSYMNWYQQKSGTSPKRWIYDTSKLASGVPAHFRGSGSGTS |
| | IgG2a | YSLTISGMEAEDAATYYCQQWSSNPFTFGSGTKLEINGGGGSGG |
| | Linker-- | GGSGGGGSGGGGSQVQLQQSGAELARPGASVKMSCKASGYTF |
| | VSVG | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSRGPTIKPCPPCKCPAPNLLGMKCLLYLAFLFIGVNCKFTI |
| | | VFPHNQKGNWKNVPSNYHYCPSSSDLNWHNDLIGTALQVKMP |
| | | KSHKAIQADGWMCHASKWVTTCDFRWYGPKYITHSIRSFTPSVE |
| | | QCKESIEQTKQGTWLNPGFPPQSCGYATVTDAEAVIVQVTPHHV |
| | | LVDEYTGEWVDSQFINGKCSNYICPTVHNSTTWHSDYKVKGLC |
| | | DSNLISMDITFFSEDGELSSLGKEGTGFRSNYFAYETGGKACKMQ |
| | | YCKHWGVRLPSGVWFEMADKDLFAAARFPECPEGSSISAPSQTS |
| | | VDVSLIQDVERILDYSLCQETWSKIRAGLPISPVDLSYLAPKNPGT |
| | | GPAFTIINGTLKYFETRYIRVDIAAPILSRMVGMISGTTTERELWD |
| | | DWAPYEDVEIGPNGVLRTSSGYKFPLYMIGHGMLDSDLHLSSKA |
| | | QVFEHPHIQDAASQLPDDESLFFGDTGLSKNPIELVEGWFSSWKS |
| | | SIASFFFIIGLIIGLFLVLRVGIHLCIKLKHTKKRQIYTDIEMNRLGK |
| 42 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | IgD | GGSGGGGSGGGGSQVQLQQSGAELARPGASVKMSCKASGYTF |
| | Linker | TRYTMHWVKQRPGQGLEWIGYINPSRGYTNYNQKFKDKATLTT |
| | (short)- | DKSSSTAYMQLSSLTSEDSAVYYCARYYDDHYCLDYWGQGTTL |
| | VSVG- | TVSSQASSVPTAQPQAEGSLAKATTAPATTRNTGRGGEEKKKEK |
| | | EKMKCLLYLAFLFIGVNCKFTIVFPHNQKGNWKNVPSNYHYCPS |
| | | SSDLNWHNDLIGTALQVKMPKSHKAIQADGWMCHASKWVTTC |
| | | DFRWYGPKYITHSIRSFTPSVEQCKESIEQTKQGTWLNPGFPPQSC |
| | | GYATVTDAEAVIVQVTPHHVLVDEYTGEWVDSQFINGKCSNYIC |
| | | PTVHNSTTWHSDYKVKGLCDSNLISMDITFFSEDGELSSLGKEGT |
| | | GFRSNYFAYETGGKACKMQYCKHWGVRLPSGVWFEMADKDLF |
| | | AAARFPECPEGSSISAPSQTSVDVSLIQDVERILDYSLCQETWSKI |
| | | RAGLPISPVDLSYLAPKNPGTGPAFTIINGTLKYFETRYIRVDIAAP |
| | | ILSRMVGMISGTTTERELWDDWAPYEDVEIGPNGVLRTSSGYKF |
| | | PLYMIGHGMLDSDLHLSSKAQVFEHPHIQDAASQLPDDESLFFG |
| | | DTGLSKNPIELVEGWFSSWKSSIASFFFIIGLIIGLFLVLRVGIHLCI |
| | | KLKHTKKRQIYTDIEMNRLGK |
| 43 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | IgD | GGSGGGGSGGGGSQVQLQQSGAELARPGASVKMSCKASGYTF |
| | linker | TRYTMHWVKQRPGQGLEWIGYINPSRGYTNYNQKFKDKATLTT |
| | (Long | DKSSSTAYMQLSSLTSEDSAVYYCARYYDDHYCLDYWGQGTTL |
| | #2) prot- | TVSSRWPESPKAQASSVPTAQPQAEGSLAKATTAPATTRNTGRG |
| | VSVG- | GEEKKKEKEKEEQEERETKTPECPMKCLLYLAFLFIGVNCKFTIV |
| | | FPHNQKGNWKNVPSNYHYCPSSSDLNWHNDLIGTALQVKMPKS |
| | | HKAIQADGWMCHASKWVTTCDFRWYGPKYITHSIRSFTPSVEQ |
| | | CKESIEQTKQGTWLNPGFPPQSCGYATVTDAEAVIVQVTPHHVL |
| | | VDEYTGEWVDSQFINGKCSNYICPTVHNSTTWHSDYKVKGLCD |
| | | SNLISMDITFFSEDGELSSLGKEGTGFRSNYFAYETGGKACKMQY |
| | | CKHWGVRLPSGVWFEMADKDLFAAARFPECPEGSSISAPSQTSV |
| | | DVSLIQDVERILDYSLCQETWSKIRAGLPISPVDLSYLAPKNPGTG |
| | | PAFTIINGTLKYFETRYIRVDIAAPILSRMVGMISGTTTERELWDD |
| | | WAPYEDVEIGPNGVLRTSSGYKFPLYMIGHGMLDSDLHLSSKAQ |
| | | VFEHPHIQDAASQLPDDESLFFGDTGLSKNPIELVEGWFSSWKSSI |
| | | ASFFFIIGLIIGLFLVLRVGIHLCIKLKHTKKRQIYTDIEMNRLGK |
| 44 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x1-- | TRYTMHWVKQRPGQGLEWIGYINPSRGYTNYNQKFKDKATLTT |
| | VSVG | DKSSSTAYMQLSSLTSEDSAVYYCARYYDDHYCLDYWGQGTTL |
| | | TVSSGGGGSMKCLLYLAFLFIGVNCKFTIVFPHNQKGNWKNVPS |
| | | NYHYCPSSSDLNWHNDLIGTALQVKMPKSHKAIQADGWMCHA |
| | | SKWVTTCDFRWYGPKYITHSIRSFTPSVEQCKESIEQTKQGTWLN |
| | | PGFPPQSCGYATVTDAEAVIVQVTPHHVLVDEYTGEWVDSQFIN |
| | | GKCSNYICPTVHNSTTWHSDYKVKGLCDSNLISMDITFFSEDGEL |
| | | SSLGKEGTGFRSNYFAYETGGKACKMQYCKHWGVRLPSGVWF |
| | | EMADKDLFAAARFPECPEGSSISAPSQTSVDVSLIQDVERILDYSL |
| | | CQETWSKIRAGLPISPVDLSYLAPKNPGTGPAFTIINGTLKYFETR |
| | | YIRVDIAAPILSRMVGMISGTTTERELWDDWAPYEDVEIGPNGVL |
| | | RTSSGYKFPLYMIGHGMLDSDLHLSSKAQVFEHPHIQDAASQLP |
| | | DDESLFFGDTGLSKNPIELVEGWFSSWKSSIASFFFIIGLIIGLFLVL |
| | | RVGIHLCIKLKHTKKRQIYTDIEMNRLGK |
| 45 | OKT3 | MEAPAQLLFLLLLWLPDTTGQIVLTQSPAIMSASPGEKVTMTCSA |
| | VL-VH)- | SSSVSYMNWYQQKSGTSPKRWIYDTSKLASGVPAHFRGSGSGTS |
| | GGGGS | YSLTISGMEAEDAATYYCQQWSSNPFTFGSGTKLEINGGGGSGG |
| | x2-- | GGSGGGGSGGGGSQVQLQQSGAELARPGASVKMSCKASGYTF |
| | VSVG | TRYTMHWVKQRPGQGLEWIGYINPSRGYTNYNQKFKDKATLTT |
| | | DKSSSTAYMQLSSLTSEDSAVYYCARYYDDHYCLDYWGQGTTL |
| | | TVSSGGGGSGGGGSMKCLLYLAFLFIGVNCKFTIVFPHNQKGN |
| | | WKNVPSNYHYCPSSSDLNWHNDLIGTALQVKMPKSHKAIQADG |
| | | WMCHASKWVTTCDFRWYGPKYITHSIRSFTPSVEQCKESIEQTK |
| | | QGTWLNPGFPPQSCGYATVTDAEAVIVQVTPHHVLVDEYTGEW |
| | | VDSQFINGKCSNYICPTVHNSTTWHSDYKVKGLCDSNLISMDITF |
| | | FSEDGELSSLGKEGTGFRSNYFAYETGGKACKMQYCKHWGVRL |
| | | PSGVWFEMADKDLFAAARFPECPEGSSISAPSQTSVDVSLIQDVE |
| | | RILDYSLCQETWSKIRAGLPISPVDLSYLAPKNPGTGPAFTIINGTL |
| | | KYFETRYIRVDIAAPILSRMVGMISGTTTERELWDDWAPYEDVEI |
| | | GPNGVLRTSSGYKFPLYMIGHGMLDSDLHLSSKAQVFEHPHIQD |
| | | AASQLPDDESLFFGDTGLSKNPIELVEGWFSSWKSSIASFFFIIGLII |
| | | GLFLVLRVGIHLCIKLKHTKKRQIYTDIEMNRLGK |
| 46 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x3 linker- | TRYTMHWVKQRPGQGLEWIGYINPSRGYTNYNQKFKDKATLTT |
| | VSVG- | DKSSSTAYMQLSSLTSEDSAVYYCARYYDDHYCLDYWGQGTTL |
| | | TVSSGGGGSGGGGSGGGGSMKCLLYLAFLFIGVNCKFTIVFPH |
| | | NQKGNWKNVPSNYHYCPSSSDLNWHNDLIGTALQVKMPKSHK |
| | | AIQADGWMCHASKWVTTCDFRWYGPKYITHSIRSFTPSVEQCKE |
| | | SIEQTKQGTWLNPGFPPQSCGYATVTDAEAVIVQVTPHHVLVDE |
| | | YTGEWVDSQFINGKCSNYICPTVHNSTTWHSDYKVKGLCDSNLI |
| | | SMDITFFSEDGELSSLGKEGTGFRSNYFAYETGGKACKMQYCKH |
| | | WGVRLPSGVWFEMADKDLFAAARFPECPEGSSISAPSQTSVDVS |
| | | LIQDVERILDYSLCQETWSKIRAGLPISPVDLSYLAPKNPGTGPAF |
| | | TIINGTLKYFETRYIRVDIAAPILSRMVGMISGTTTERELWDDWA |
| | | PYEDVEIGPNGVLRTSSGYKFPLYMIGHGMLDSDLHLSSKAQVF |
| | | EHPHIQDAASQLPDDESLFFGDTGLSKNPIELVEGWFSSWKSSIAS |
| | | FFFIIGLIIGLFLVLRVGIHLCIKLKHTKKRQIYTDIEMNRLGK |
| 47 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x4 linker-- | TRYTMHWVKQRPGQGLEWIGYINPSRGYTNYNQKFKDKATLTT |
| | VSVG | DKSSSTAYMQLSSLTSEDSAVYYCARYYDDHYCLDYWGQGTTL |
| | | TVSSGGGGSGGGGSGGGGSGGGGSMKCLLYLAFLFIGVNCKF |
| | | TIVFPHNQKGNWKNVPSNYHYCPSSSDLNWHNDLIGTALQVKM |
| | | PKSHKAIQADGWMCHASKWVTTCDFRWYGPKYITHSIRSFTPSV |
| | | EQCKESIEQTKQGTWLNPGFPPQSCGYATVTDAEAVIVQVTPHH |
| | | VLVDEYTGEWVDSQFINGKCSNYICPTVHNSTTWHSDYKVKGL |
| | | CDSNLISMDITFFSEDGELSSLGKEGTGFRSNYFAYETGGKACKM |
| | | QYCKHWGVRLPSGVWFEMADKDLFAAARFPECPEGSSISAPSQT |
| | | SVDVSLIQDVERILDYSLCQETWSKIRAGLPISPVDLSYLAPKNPG |
| | | TGPAFTIINGTLKYFETRYIRVDIAAPILSRMVGMISGTTTERELW |
| | | DDWAPYEDVEIGPNGVLRTSSGYKFPLYMIGHGMLDSDLHLSSK |
| | | AQVFEHPHIQDAASQLPDDESLFFGDTGLSKNPIELVEGWFSSWK |
| | | SSIASFFFIIGLIIGLFLVLRVGIHLCIKLKHTKKRQIYTDIEMNRLG |
| | | K |
| 48 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x2 linker- | TRYTMHWVKQRPGQGLEWIGYINPSRGYTNYNQKFKDKATLTT |
| | VSVG | DKSSSTAYMQLSSLTSEDSAVYYCARYYDDHYCLDYWGQGTTL |
| | (K47Q/ | TVSSGGGGSGGGGSMKCLLYLAFLFIGVNCKFTIVFPHNQKGN |
| | R354A) | WKNVPSNYHYCPSSSDLNWHNDLIGTALQVKMPQSHKAIQADG |
| | | WMCHASKWVTTCDFRWYGPKYITHSIRSFTPSVEQCKESIEQTK |
| | | QGTWLNPGFPPQSCGYATVTDAEAVIVQVTPHHVLVDEYTGEW |
| | | VDSQFINGKCSNYICPTVHNSTTWHSDYKVKGLCDSNLISMDITF |
| | | FSEDGELSSLGKEGTGFRSNYFAYETGGKACKMQYCKHWGVRL |
| | | PSGVWFEMADKDLFAAARFPECPEGSSISAPSQTSVDVSLIQDVE |
| | | RILDYSLCQETWSKIRAGLPISPVDLSYLAPKNPGTGPAFTIINGTL |
| | | KYFETRYIRVDIAAPILSRMVGMISGTTTEAELWDDWAPYEDVEI |
| | | GPNGVLRTSSGYKFPLYMIGHGMLDSDLHLSSKAQVFEHPHIQD |
| | | AASQLPDDESLFFGDTGLSKNPIELVEGWFSSWKSSIASFFFIIGLII |
| | | GLFLVLRVGIHLCIKLKHTKKRQIYTDIEMNRLGK |
| 49 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x3 linker-- | TRYTMHWVKQRPGQGLEWIGYINPSRGYTNYNQKFKDKATLTT |
| | VSVG | DKSSSTAYMQLSSLTSEDSAVYYCARYYDDHYCLDYWGQGTTL |
| | (K47Q/ | TVSSGGGGSGGGGSGGGGSMKCLLYLAFLFIGVNCKFTIVFPH |
| | R354A) | NQKGNWKNVPSNYHYCPSSSDLNWHNDLIGTALQVKMPQSHK |
| | | AIQADGWMCHASKWVTTCDFRWYGPKYITHSIRSFTPSVEQCKE |
| | | SIEQTKQGTWLNPGFPPQSCGYATVTDAEAVIVQVTPHHVLVDE |
| | | YTGEWVDSQFINGKCSNYICPTVHNSTTWHSDYKVKGLCDSNLI |
| | | SMDITFFSEDGELSSLGKEGTGFRSNYFAYETGGKACKMQYCKH |
| | | WGVRLPSGVWFEMADKDLFAAARFPECPEGSSISAPSQTSVDVS |
| | | LIQDVERILDYSLCQETWSKIRAGLPISPVDLSYLAPKNPGTGPAF |
| | | TIINGTLKYFETRYIRVDIAAPILSRMVGMISGTTTEAELWDDWA |
| | | PYEDVEIGPNGVLRTSSGYKFPLYMIGHGMLDSDLHLSSKAQVF |
| | | EHPHIQDAASQLPDDESLFFGDTGLSKNPIELVEGWFSSWKSSIAS |
| | | FFFIIGLIIGLFLVLRVGIHLCIKLKHTKKRQIYTDIEMNRLGK |
| 50 | OKT3 | MEAPAQLLFLLLLWLPDTTGQIVLTQSPAIMSASPGEKVTMTCSA |
| | (VL- | SSSVSYMNWYQQKSGTSPKRWIYDTSKLASGVPAHFRGSGSGTS |
| | VH)- | YSLTISGMEAEDAATYYCQQWSSNPFTFGSGTKLEINGGGGSGG |
| | GGGGS | GGSGGGGSGGGGSQVQLQQSGAELARPGASVKMSCKASGYTF |
| | x4 linker-- | TRYTMHWVKQRPGQGLEWIGYINPSRGYTNYNQKFKDKATLTT |
| | VSVG | DKSSSTAYMQLSSLTSEDSAVYYCARYYDDHYCLDYWGQGTTL |
| | (K47Q/ | TVSSGGGGSGGGGSGGGGSGGGGSMKCLLYLAFLFIGVNCKF |
| | R354A) | TIVFPHNQKGNWKNVPSNYHYCPSSSDLNWHNDLIGTALQVKM |
| | | PQSHKAIQADGWMCHASKWVTTCDFRWYGPKYITHSIRSFTPSV |
| | | EQCKESIEQTKQGTWLNPGFPPQSCGYATVTDAEAVIVQVTPHH |
| | | VLVDEYTGEWVDSQFINGKCSNYICPTVHNSTTWHSDYKVKGL |
| | | CDSNLISMDITFFSEDGELSSLGKEGTGFRSNYFAYETGGKACKM |
| | | QYCKHWGVRLPSGVWFEMADKDLFAAARFPECPEGSSISAPSQT |
| | | SVDVSLIQDVERILDYSLCQETWSKIRAGLPISPVDLSYLAPKNPG |
| | | TGPAFTIINGTLKYFETRYIRVDIAAPILSRMVGMISGTTTEAELW |
| | | DDWAPYEDVEIGPNGVLRTSSGYKFPLYMIGHGMLDSDLHLSSK |
| | | AQVFEHPHIQDAASQLPDDESLFFGDTGLSKNPIELVEGWFSSWK |
| | | SSIASFFFIIGLIIGLFLVLRVGIHLCIKLKHTKKRQIYTDIEMNRLG |
| | | K |
| 51 | OKT3 | MEAPAQLLFLLLLWLPDTTGQIVLTQSPAIMSASPGEKVTMTCSA |
| | (VL-VH)- - | SSSVSYMNWYQQKSGTSPKRWIYDTSKLASGVPAHFRGSGSGTS |
| | CD8a- | YSLTISGMEAEDAATYYCQQWSSNPFTFGSGTKLEINGGGGSGG |
| | VSVG | GGSGGGGSGGGGSQVQLQQSGAELARPGASVKMSCKASGYTF |
| | (K47Q/ | TRYTMHWVKQRPGQGLEWIGYINPSRGYTNYNQKFKDKATLTT |
| | R354A) | DKSSSTAYMQLSSLTSEDSAVYYCARYYDDHYCLDYWGQGTTL |
| | | TVSSKPTTTPAPRPPTPAPTIASQPLSLRPEACRPAAGGAVHTRGL |
| | | MKCLLYLAFLFIGVNCKFTIVFPHNQKGNWKNVPSNYHYCPSSS |
| | | DLNWHNDLIGTALQVKMPQSHKAIQADGWMCHASKWVTTCDF |
| | | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTEAELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 52 | OKT3 | MEAPAQLLFLLLLWLPDTTGQVQLQQSGAELARPGASVKMSCK |
| | V_{H}-V_{L} | ASGYTFTRYTMHWVKQRPGQGLEWIGYINPSRGYTNYNQKFKD |
| | prot | KATLTTDKSSSTAYMQLSSLTSEDSAVYYCARYYDDHYCLDYW |
| | | GQGTTLTVSSGGGGSGGGGSGGGGSQIVLTQSPAIMSASPGEK |
| | | VTMTCSASSSVSYMNWYQQKSGTSPKRWIYDTSKLASGVPAHF |
| | | RGSGSGTSYSLTISGMEAEDAATYYCQQWSSNPFTFGSGTKLEIN |
| 53 | OKT3 | atggaggcccccgcccaactcctcttcctcctgttgctctggctgccggacaccaccggtcaagtgcag |
| | V_{H}-V_{L} | ctgcagcaatcaggggccgagctcgccaggcctggcgcatcagttaaaatgtcatgtaaggcatcag |
| | DNA | ggtacacctttacaagatacacaatgcactgggtgaagcagaggcccggtcagggtctggaatggatc |
| | | ggctatatcaatcctagcagaggttatacaaactacaatcaaaaatttaaggataaagccacattgacaa |
| | | ctgacaagagtagttccactgcttatatgcagctgagcagcctcacgtccgaggacagtgcagtttatta |
| | | ttgtgcgcgatattacgacgaccattattgcttggattactggggacagggcacgacgctcacagtcagt |
| | | tctggcggtgggggcagtggggggggtgggtctggaggcggaggctcccagatagtcttgacgcag |
| | | tcaccggctataatgtcagcaagtcctggagagaaagtcacaatgacttgcagtgcgtccagtagtgtta |
| | | gttacatgaactggtaccagcaaaaaagtggtacaagccccaagagatggatctacgacaccagcaa |
| | | gctggcaagtggtgtaccagcgcattttcgaggctctgggagtgggacgtcttactctctcacgattagc |
| | | ggcatggaagcggaagacgcggcaacatattactgtcaacaatggtcttccaatcctttcacttttggttc |
| | | aggcacaaagctcgagataaatagagctgacacggctccgggcggcggg |
| 54 | 500A2 | MEAPAQLLFLLLLWLPDTTGDIVLTQTPATLSLIPGERVTMTCKT |
| | scFv | SQNIGTILHWYHQKPKEAPRALIKYASQSIPGIPSRFSGSGSETDFT |
| | Mouse | LSINNLEPDDIGIYYCQQSRSWPVTFGPGTKLEIKGGGGSGGGGS |
| | prot | GGGGSGGGGSQVKLQQSGSELGKPGASVKLSCKTSGYIFTDHYI |
| | | SWVKQKPGESLQWIGNVYGGNGGTSYNQKFQGKATLTVDKISS |
| | | TAYMELSSLTSEDSAIYYCARRPVATGHAMDYWGQGIQVTVSS |
| 55 | 500A2 | Atggaggcacctgcacaacttctgttcttgctcttgctttggctccccgacacaacaggtgatatagtcct |
| | scFv | tactcagacccctgcaacgctttccttgatcccgggagagcgagtaactatgacctgcaagactagtca |
| | Mouse | gaatatcggtacaatccttcactggtaccatcagaagcccaaagaggctcctagggcacttatcaaatat |
| | prot | gcctctcagagtattccaggtatcccttcaagattctccgggtcagggtccgagacggactttacactttc |
| | | aatcaacaatttggagccggacgatataggaatatattattgccaacaatctcggagttggcccgtcacg |
| | | tttgggccaggaacaaagctcgaaataaaaggaggtggaggtagcggcggagggggatccggtggt |
| | | ggcggttccgggggtggtggatctcaggtaaaactccagcaatctggatcagagttgggtaaaccgg |
| | | gagcctccgtgaagttgtcttgtaaaacgagtgggtatatattcacggatcattacatctcctgggtgaag |
| | | caaaagccaggtgaaagtcttcagtggatcggaaacgtctatggaggtaacgggggcacgtcttataa |
| | | ccaaaagtttcaaggcaaggcaactttgaccgtggataagatatctagcactgcctatatggagctctcc |
| | | tctcttacctctgaggattcagccatttattattgtgcacgaaggccggtagctactggtcatgctatggac |
| | | tattgggggcagggtatacaggtaactgtatcttca |
| 56 | Linker | Ggagggggaggaagt |
| | DNA | |
| 57 | VSVG | MKCLLYLAFLFIGVNC |
| | signal | |
| | peptide | |
| | (prot) | |
| 58 | VSVG | MKCLLYLAFLFIGVNCKFTIVFPHNQKGNWKNVPSNYHYCPSSS |
| | extra- | DLNWHNDLIGTALQVKMPKSHKAIQADGWMCHASKWVTTCDF |
| | cellular | RWYGPKYITHSIRSFTPSVEQCKESIEQTKQGTWLNPGFPPQSCG |
| | domain | YATVTDAEAVIVQVTPHHVLVDEYTGEWVDSQFINGKCSNYICP |
| | (prot) | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSS |
| 59 | VSVG | KFTIVFPHNQKGNWKNVPSNYHYCPSSSDLNWHNDLIGTALQV |
| | extra- | KMPKSHKAIQADGWMCHASKWVTTCDFRWYGPKYITHSIRSFT |
| | cellular | PSVEQCKESIEQTKQGTWLNPGFPPQSCGYATVTDAEAVIVQVTP |
| | domain | HHVLVDEYTGEWVDSQFINGKCSNYICPTVHNSTTWHSDYKVK |
| | no SP | GLCDSNLISMDITFFSEDGELSSLGKEGTGFRSNYFAYETGGKAC |
| | (prot) | KMQYCKHWGVRLPSGVWFEMADKDLFAAARFPECPEGSSISAP |
| | | SQTSVDVSLIQDVERILDYSLCQETWSKIRAGLPISPVDLSYLAPK |
| | | NPGTGPAFTIINGTLKYFETRYIRVDIAAPILSRMVGMISGTTTERE |
| | | LWDDWAPYEDVEIGPNGVLRTSSGYKFPLYMIGHGMLDSDLHL |
| | | SSKAQVFEHPHIQDAASQLPDDESLFFGDTGLSKNPIELVEGWFS |
| | | S |
| 60 | VSVG | KFTIVFPHNQKGNWKNVPSNYHYCPSSSDLNWHNDLIGTALQV |
| | no SP | KMPKSHKAIQADGWMCHASKWVTTCDFRWYGPKYITHSIRSFT |
| | (prot) | PSVEQCKESIEQTKQGTWLNPGFPPQSCGYATVTDAEAVIVQVTP |
| | | HHVLVDEYTGEWVDSQFINGKCSNYICPTVHNSTTWHSDYKVK |
| | | GLCDSNLISMDITFFSEDGELSSLGKEGTGFRSNYFAYETGGKAC |
| | | KMQYCKHWGVRLPSGVWFEMADKDLFAAARFPECPEGSSISAP |
| | | SQTSVDVSLIQDVERILDYSLCQETWSKIRAGLPISPVDLSYLAPK |
| | | NPGTGPAFTIINGTLKYFETRYIRVDIAAPILSRMVGMISGTTTERE |
| | | LWDDWAPYEDVEIGPNGVLRTSSGYKFPLYMIGHGMLDSDLHL |
| | | SSKAQVFEHPHIQDAASQLPDDESLFFGDTGLSKNPIELVEGWFS |
| | | SWKSSIASFFFIIGLIIGLFLVLRVGIHLCIKLKHTKKRQIYTDIEMN |
| | | RLGK |
| 61 | VSVG | MKCLLYLAFLFIGVNCKFTIVFPHNQKGNWKNVPSNYHYCPSSS |
| | extra- | DLNWHNDLIGTALQVKMPKSHKAIQADGWMCHASKWVTTCDF |
| | cellular | RWYGPKYITHSIRSFTPSVEQCKESIEQTKQGTWLNPGFPPQSCG |
| | domain | YATVTDAEAVIVQVTPHHVLVDEYTGEWVDSQFINGKCSNYICP |
| | (alt1) | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWK |
| 62 | VSVG | MKCLLYLAFLFIGVNCKFTIVFPHNQKGNWKNVPSNYHYCPSSS |
| | extra- | DLNWHNDLIGTALQVKMPKSHKAIQADGWMCHASKWVTTCDF |
| | cellular | RWYGPKYITHSIRSFTPSVEQCKESIEQTKQGTWLNPGFPPQSCG |
| | domain | YATVTDAEAVIVQVTPHHVLVDEYTGEWVDSQFINGKCSNYICP |
| | (alt2) | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSS |
| 63 | VSVG | MKCLLYLAFLFIGVNCKFTIVFPHNQKGNWKNVPSNYHYCPSSS |
| | extra- | DLNWHNDLIGTALQVKMPKSHKAIQADGWMCHASKWVTTCDF |
| | cellular | RWYGPKYITHSIRSFTPSVEQCKESIEQTKQGTWLNPGFPPQSCG |
| | domain | YATVTDAEAVIVQVTPHHVLVDEYTGEWVDSQFINGKCSNYICP |
| | (alt3) | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIAS |
| 64 | OKT3 | MEAPAQLLFLLLLWLPDTTGQVQLQQSGAELARPGASVKMSCK |
| | (VH-VL) | ASGYTFTRYTMHWVKQRPGQGLEWIGYINPSRGYTNYNQKFKD |
| | VSVG- | KATLTTDKSSSTAYMQLSSLTSEDSAVYYCARYYDDHYCLDYW |
| | | GQGTTLTVSSGGGGSGGGGSGGGGSQIVLTQSPAIMSASPGEK |
| | | VTMTCSASSSVSYMNWYQQKSGTSPKRWIYDTSKLASGVPAHF |
| | | RGSGSGTSYSLTISGMEAEDAATYYCQQWSSNPFTFGSGTKLEIN |
| | | MKCLLYLAFLFIGVNCKFTIVFPHNQKGNWKNVPSNYHYCPSSS |
| | | DLNWHNDLIGTALQVKMPKSHKAIQADGWMCHASKWVTTCDF |
| | | RWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 65 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | CD8a- | GQGTTLTVSSGGGGSGGGGSGGGGSQIVLTQSPAIMSASPGEK |
| | VSVG | VTMTCSASSSVSYMNWYQQKSGTSPKRWIYDTSKLASGVPAHF |
| | | RGSGSGTSYSLTISGMEAEDAATYYCQQWSSNPFTFGSGTKLEIN |
| | | KPTTTPAPRPPTPAPTIASQPLSLRPEACRPAAGGAVHTRGLM |
| | | KCLLYLAFLFIGVNCKFTIVFPHNQKGNWKNVPSNYHYCPSSSD |
| | | LNWHNDLIGTALQVKMPKSHKAIQADGWMCHASKWVTTCDFR |
| | | WYGPKYITHSIRSFTPSVEQCKESIEQTKQGTWLNPGFPPQSCGY |
| | | ATVTDAEAVIVQVTPHHVLVDEYTGEWVDSQFINGKCSNYICPT |
| | | VHNSTTWHSDYKVKGLCDSNLISMDITFFSEDGELSSLGKEGTGF |
| | | RSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 66 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | IgD | GQGTTLTVSSGGGGSGGGGSGGGGSQIVLTQSPAIMSASPGEK |
| | Hinge- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | | SPKAQASSVPTAQPQAEGSLAKATTAPATTRNTGRGGEEKK |
| | | KEKEKEEQEERETKTPEMKCLLYLAFLFIGVNCKFTIVFPHNQ |
| | | KGNWKNVPSNYHYCPSSSDLNWHNDLIGTALQVKMPKSHKAIQ |
| | | ADGWMCHASKWVTTCDFRWYGPKYITHSIRSFTPSVEQCKESIE |
| | | QTKQGTWLNPGFPPQSCGYATVTDAEAVIVQVTPHHVLVDEYT |
| | | GEWVDSQFINGKCSNYICPTVHNSTTWHSDYKVKGLCDSNLISM |
| | | DITFFSEDGELSSLGKEGTGFRSNYFAYETGGKACKMQYCKHW |
| | | GVRLPSGVWFEMADKDLFAAARFPECPEGSSISAPSQTSVDVSLI |
| | | QDVERILDYSLCQETWSKIRAGLPISPVDLSYLAPKNPGTGPAFTI |
| | | INGTLKYFETRYIRVDIAAPILSRMVGMISGTTTERELWDDWAPY |
| | | EDVEIGPNGVLRTSSGYKFPLYMIGHGMLDSDLHLSSKAQVFEH |
| | | PHIQDAASQLPDDESLFFGDTGLSKNPIELVEGWFSSWKSSIASFF |
| | | FIIGLIIGLFLVLRVGIHLCIKLKHTKKRQIYTDIEMNRLGK |
| 67 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | IgG1 | GQGTTLTVSSGGGGSGGGGSGGGGSQIVLTQSPAIMSASPGEK |
| | hinge- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | | DKTHTCPPCPAPELLGGMKCLLYLAFLFIGVNCKFTIVFPHNQK |
| | | GNWKNVPSNYHYCPSSSDLNWHNDLIGTALQVKMPKSHKAIQA |
| | | DGWMCHASKWVTTCDFRWYGPKYITHSIRSFTPSVEQCKESIEQ |
| | | TKQGTWLNPGFPPQSCGYATVTDAEAVIVQVTPHHVLVDEYTG |
| | | EWVDSQFINGKCSNYICPTVHNSTTWHSDYKVKGLCDSNLISMD |
| | | ITFFSEDGELSSLGKEGTGFRSNYFAYETGGKACKMQYCKHWG |
| | | VRLPSGVWFEMADKDLFAAARFPECPEGSSISAPSQTSVDVSLIQ |
| | | DVERILDYSLCQETWSKIRAGLPISPVDLSYLAPKNPGTGPAFTII |
| | | NGTLKYFETRYIRVDIAAPILSRMVGMISGTTTERELWDDWAPY |
| | | EDVEIGPNGVLRTSSGYKFPLYMIGHGMLDSDLHLSSKAQVFEH |
| | | PHIQDAASQLPDDESLFFGDTGLSKNPIELVEGWFSSWKSSIASFF |
| | | FIIGLIIGLFLVLRVGIHLCIKLKHTKKRQIYTDIEMNRLGK |
| 68 | OKT3 | MEAPAQLLFLLLLWLPDTTGQVQLQQSGAELARPGASVKMSCK |
| | (VH-VL) | ASGYTFTRYTMHWVKQRPGQGLEWIGYINPSRGYTNYNQKFKD |
| | IgG2a | KATLTTDKSSSTAYMQLSSLTSEDSAVYYCARYYDDHYCLDYW |
| | Linker-- | GQGTTLTVSSGGGGSGGGGSGGGGSQIVLTQSPAIMSASPGEK |
| | VSVG | VTMTCSASSSVSYMNWYQQKSGTSPKRWIYDTSKLASGVPAHF |
| | | RGSGSGTSYSLTISGMEAEDAATYYCQQWSSNPFTFGSGTKLEIN |
| | | RGPTIKPCPPCKCPAPNLLGMKCLLYLAFLFIGVNCKFTIVFPH |
| | | NQKGNWKNVPSNYHYCPSSSDLNWHNDLIGTALQVKMPKSHK |
| | | AIQADGWMCHASKWVTTCDFRWYGPKYITHSIRSFTPSVEQCKE |
| | | SIEQTKQGTWLNPGFPPQSCGYATVTDAEAVIVQVTPHHVLVDE |
| | | YTGEWVDSQFINGKCSNYICPTVHNSTTWHSDYKVKGLCDSNLI |
| | | SMDITFFSEDGELSSLGKEGTGFRSNYFAYETGGKACKMQYCKH |
| | | WGVRLPSGVWFEMADKDLFAAARFPECPEGSSISAPSQTSVDVS |
| | | LIQDVERILDYSLCQETWSKIRAGLPISPVDLSYLAPKNPGTGPAF |
| | | TIINGTLKYFETRYIRVDIAAPILSRMVGMISGTTTERELWDDWA |
| | | PYEDVEIGPNGVLRTSSGYKFPLYMIGHGMLDSDLHLSSKAQVF |
| | | EHPHIQDAASQLPDDESLFFGDTGLSKNPIELVEGWFSSWKSSIAS |
| | | FFFIIGLIIGLFLVLRVGIHLCIKLKHTKKRQIYTDIEMNRLGK |
| 69 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | IgD | GQGTTLTVSSGGGGSGGGGSGGGGSQIVLTQSPAIMSASPGEK |
| | Linker | VTMTCSASSSVSYMNWYQQKSGTSPKRWIYDTSKLASGVPAHF |
| | (short)- | RGSGSGTSYSLTISGMEAEDAATYYCQQWSSNPFTFGSGTKLEIN |
| | VSVG- | QASSVPTAQPQAEGSLAKATTAPATTRNTGRGGEEKKKEKE |
| | | KMKCLLYLAFLFIGVNCKFTIVFPHNQKGNWKNVPSNYHYCPSS |
| | | SDLNWHNDLIGTALQVKMPKSHKAIQADGWMCHASKWVTTCD |
| | | FRWYGPKYITHSIRSFTPSVEQCKESIEQTKQGTWLNPGFPPQSCG |
| | | YATVTDAEAVIVQVTPHHVLVDEYTGEWVDSQFINGKCSNYICP |
| | | TVHNSTTWHSDYKVKGLCDSNLISMDITFFSEDGELSSLGKEGTG |
| | | FRSNYFAYETGGKACKMQYCKHWGVRLPSGVWFEMADKDLFA |
| | | AARFPECPEGSSISAPSQTSVDVSLIQDVERILDYSLCQETWSKIR |
| | | AGLPISPVDLSYLAPKNPGTGPAFTIINGTLKYFETRYIRVDIAAPI |
| | | LSRMVGMISGTTTERELWDDWAPYEDVEIGPNGVLRTSSGYKFP |
| | | LYMIGHGMLDSDLHLSSKAQVFEHPHIQDAASQLPDDESLFFGD |
| | | TGLSKNPIELVEGWFSSWKSSIASFFFIIGLIIGLFLVLRVGIHLCIK |
| | | LKHTKKRQIYTDIEMNRLGK |
| 70 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | IgD | GQGTTLTVSSGGGGSGGGGSGGGGSQIVLTQSPAIMSASPGEK |
| | linker | VTMTCSASSSVSYMNWYQQKSGTSPKRWIYDTSKLASGVPAHF |
| | (Long | RGSGSGTSYSLTISGMEAEDAATYYCQQWSSNPFTFGSGTKLEIN |
| | #2) prot- | RWPESPKAQASSVPTAQPQAEGSLAKATTAPATTRNTGRGG |
| | VSVG- | EEKKKEKEKEEQEERETKTPECPMKCLLYLAFLFIGVNCKFTI |
| | | VFPHNQKGNWKNVPSNYHYCPSSSDLNWHNDLIGTALQVKMP |
| | | KSHKAIQADGWMCHASKWVTTCDFRWYGPKYITHSIRSFTPSVE |
| | | QCKESIEQTKQGTWLNPGFPPQSCGYATVTDAEAVIVQVTPHHV |
| | | LVDEYTGEWVDSQFINGKCSNYICPTVHNSTTWHSDYKVKGLC |
| | | DSNLISMDITFFSEDGELSSLGKEGTGFRSNYFAYETGGKACKMQ |
| | | YCKHWGVRLPSGVWFEMADKDLFAAARFPECPEGSSISAPSQTS |
| | | VDVSLIQDVERILDYSLCQETWSKIRAGLPISPVDLSYLAPKNPGT |
| | | GPAFTIINGTLKYFETRYIRVDIAAPILSRMVGMISGTTTERELWD |
| | | DWAPYEDVEIGPNGVLRTSSGYKFPLYMIGHGMLDSDLHLSSKA |
| | | QVFEHPHIQDAASQLPDDESLFFGDTGLSKNPIELVEGWFSSWKS |
| | | SIASFFFIIGLIIGLFLVLRVGIHLCIKLKHTKKRQIYTDIEMNRLGK |
| 71 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x1-- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | | GGGGSMKCLLYLAFLFIGVNCKFTIVFPHNQKGNWKNVPSNYH |
| | | YCPSSSDLNWHNDLIGTALQVKMPKSHKAIQADGWMCHASKW |
| | | VTTCDFRWYGPKYITHSIRSFTPSVEQCKESIEQTKQGTWLNPGF |
| | | PPQSCGYATVTDAEAVIVQVTPHHVLVDEYTGEWVDSQFINGKC |
| | | SNYICPTVHNSTTWHSDYKVKGLCDSNLISMDITFFSEDGELSSL |
| | | GKEGTGFRSNYFAYETGGKACKMQYCKHWGVRLPSGVWFEMA |
| | | DKDLFAAARFPECPEGSSISAPSQTSVDVSLIQDVERILDYSLCQE |
| | | TWSKIRAGLPISPVDLSYLAPKNPGTGPAFTIINGTLKYFETRYIR |
| | | VDIAAPILSRMVGMISGTTTERELWDDWAPYEDVEIGPNGVLRT |
| | | SSGYKFPLYMIGHGMLDSDLHLSSKAQVFEHPHIQDAASQLPDD |
| | | ESLFFGDTGLSKNPIELVEGWFSSWKSSIASFFFIIGLIIGLFLVLRV |
| | | GIHLCIKLKHTKKRQIYTDIEMNRLGK |
| 72 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x2- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | | GGGGSGGGGSMKCLLYLAFLFIGVNCKFTIVFPHNQKGNWKN |
| | | VPSNYHYCPSSSDLNWHNDLIGTALQVI<MPI<SHI<AIQADGWMC |
| | | HASKWVTTCDFRWYGPKYITHSIRSFTPSVEQCKESIEQTKQGT |
| | | WLNPGFPPQSCGYATVTDAEAVIVQVTPHHVLVDEYTGEWVDS |
| | | QFINGKCSNYICPTVHNSTTWHSDYKVKGLCDSNLISMDITFFSE |
| | | DGELSSLGKEGTGFRSNYFAYETGGKACKMQYCKHWGVRLPSG |
| | | VWFEMADKDLFAAARFPECPEGSSISAPSQTSVDVSLIQDVERIL |
| | | DYSLCQETWSKIRAGLPISPVDLSYLAPKNPGTGPAFTIINGTLKY |
| | | FETRYIRVDIAAPILSRMVGMISGTTTERELWDDWAPYEDVEIGP |
| | | NGVLRTSSGYKFPLYMIGHGMLDSDLHLSSKAQVFEHPHIQDAA |
| | | SQLPDDESLFFGDTGLSKNPIELVEGWFSSWKSSIASFFFIIGLIIGL |
| | | FLVLRVGIHLCIKLKHTKKRQIYTDIEMNRLGK |
| 73 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x3 linker- | VTMTCSASSSVSYMNWYQQKSGTSPKRWIYDTSKLASGVPAHF |
| | VSVG- | RGSGSGTSYSLTISGMEAEDAATYYCQQWSSNPFTFGSGTKLEIN |
| | | GGGGSGGGGSGGGGSMKCLLYLAFLFIGVNCKFTIVFPHNQKG |
| | | NWKNVPSNYHYCPSSSDLNWHNDLIGTALQVKMPKSHKAIQAD |
| | | GWMCHASKWVTTCDFRWYGPKYITHSIRSFTPSVEQCKESIEQT |
| | | KQGTWLNPGFPPQSCGYATVTDAEAVIVQVTPHHVLVDEYTGE |
| | | WVDSQFINGKCSNYICPTVHNSTTWHSDYKVKGLCDSNLISMDI |
| | | TFFSEDGELSSLGKEGTGFRSNYFAYETGGKACKMQYCKHWGV |
| | | RLPSGVWFEMADKDLFAAARFPECPEGSSISAPSQTSVDVSLIQD |
| | | VERILDYSLCQETWSKIRAGLPISPVDLSYLAPKNPGTGPAFTIIN |
| | | GTLKYFETRYIRVDIAAPILSRMVGMISGTTTERELWDDWAPYE |
| | | DVEIGPNGVLRTSSGYKFPLYMIGHGMLDSDLHLSSKAQVFEHP |
| | | HIQDAASQLPDDESLFFGDTGLSKNPIELVEGWFSSWKSSIASFFF |
| | | IIGLIIGLFLVLRVGIHLCIKLKHTKKRQIYTDIEMNRLGK |
| 74 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x4 linker-- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | | GGGGSGGGGSGGGGSGGGGSMKCLLYLAFLFIGVNCKFTIVFP |
| | | HNQKGNWKNVPSNYHYCPSSSDLNWHNDLIGTALQVKMPKSH |
| | | KAIQADGWMCHASKWVTTCDFRWYGPKYITHSIRSFTPSVEQC |
| | | KESIEQTKQGTWLNPGFPPQSCGYATVTDAEAVIVQVTPHHVLV |
| | | DEYTGEWVDSQFINGKCSNYICPTVHNSTTWHSDYKVKGLCDS |
| | | NLISMDITFFSEDGELSSLGKEGTGFRSNYFAYETGGKACKMQY |
| | | CKHWGVRLPSGVWFEMADKDLFAAARFPECPEGSSISAPSQTSV |
| | | DVSLIQDVERILDYSLCQETWSKIRAGLPISPVDLSYLAPKNPGTG |
| | | PAFTIINGTLKYFETRYIRVDIAAPILSRMVGMISGTTTERELWDD |
| | | WAPYEDVEIGPNGVLRTSSGYKFPLYMIGHGMLDSDLHLSSKAQ |
| | | VFEHPHIQDAASQLPDDESLFFGDTGLSKNPIELVEGWFSSWKSSI |
| | | ASFFFIIGLIIGLFLVLRVGIHLCIKLKHTKKRQIYTDIEMNRLGK |
| 75 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x2 linker- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | (K47Q/ | GGGGSGGGGSMKCLLYLAFLFIGVNCKFTIVFPHNQKGNWKN |
| | R354A) | VPSNYHYCPSSSDLNWHNDLIGTALQVKMPQSHKAIQADGWMC |
| | | HASKWVTTCDFRWYGPKYITHSIRSFTPSVEQCKESIEQTKQGT |
| | | WLNPGFPPQSCGYATVTDAEAVIVQVTPHHVLVDEYTGEWVDS |
| | | QFINGKCSNYICPTVHNSTTWHSDYKVKGLCDSNLISMDITFFSE |
| | | DGELSSLGKEGTGFRSNYFAYETGGKACKMQYCKHWGVRLPSG |
| | | VWFEMADKDLFAAARFPECPEGSSISAPSQTSVDVSLIQDVERIL |
| | | DYSLCQETWSKIRAGLPISPVDLSYLAPKNPGTGPAFTIINGTLKY |
| | | FETRYIRVDIAAPILSRMVGMISGTTTEAELWDDWAPYEDVEIGP |
| | | NGVLRTSSGYKFPLYMIGHGMLDSDLHLSSKAQVFEHPHIQDAA |
| | | SQLPDDESLFFGDTGLSKNPIELVEGWFSSWKSSIASFFFIIGLIIGL |
| | | FLVLRVGIHLCIKLKHTKKRQIYTDIEMNRLGK |
| 76 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x3 linker-- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | (K47Q/ | GGGGSGGGGSGGGGSMKCLLYLAFLFIGVNCKFTIVFPHNQKG |
| | R354A) | NWKNVPSNYHYCPSSSDLNWHNDLIGTALQVKMPQSHKAIQAD |
| | | GWMCHASKWVTTCDFRWYGPKYITHSIRSFTPSVEQCKESIEQT |
| | | KQGTWLNPGFPPQSCGYATVTDAEAVIVQVTPHHVLVDEYTGE |
| | | WVDSQFINGKCSNYICPTVHNSTTWHSDYKVKGLCDSNLISMDI |
| | | TFFSEDGELSSLGKEGTGFRSNYFAYETGGKACKMQYCKHWGV |
| | | RLPSGVWFEMADKDLFAAARFPECPEGSSISAPSQTSVDVSLIQD |
| | | VERILDYSLCQETWSKIRAGLPISPVDLSYLAPKNPGTGPAFTIIN |
| | | GTLKYFETRYIRVDIAAPILSRMVGMISGTTTEAELWDDWAPYE |
| | | DVEIGPNGVLRTSSGYKFPLYMIGHGMLDSDLHLSSKAQVFEHP |
| | | HIQDAASQLPDDESLFFGDTGLSKNPIELVEGWFSSWKSSIASFFF |
| | | IIGLIIGLFLVLRVGIHLCIKLKHTKKRQIYTDIEMNRLGK |
| 77 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- | KATLTTDKSSSTAYMQLSSLTSEDSAVYYCARYYDDHYCLDYW |
| | GGGGS | GQGTTLTVSSGGGGSGGGGSGGGGSQIVLTQSPAIMSASPGEK |
| | x4 linker-- | VTMTCSASSSVSYMNWYQQKSGTSPKRWIYDTSKLASGVPAHF |
| | VSVG | RGSGSGTSYSLTISGMEAEDAATYYCQQWSSNPFTFGSGTKLEIN |
| | (K47Q/ | GGGGSGGGGSGGGGSGGGGSMKCLLYLAFLFIGVNCKFTIVFP |
| | R354A) | HNQKGNWKNVPSNYHYCPSSSDLNWHNDLIGTALQVKMPQSH |
| | | KAIQADGWMCHASKWVTTCDFRWYGPKYITHSIRSFTPSVEQC |
| | | KESIEQTKQGTWLNPGFPPQSCGYATVTDAEAVIVQVTPHHVLV |
| | | DEYTGEWVDSQFINGKCSNYICPTVHNSTTWHSDYKVKGLCDS |
| | | NLISMDITFFSEDGELSSLGKEGTGFRSNYFAYETGGKACKMQY |
| | | CKHWGVRLPSGVWFEMADKDLFAAARFPECPEGSSISAPSQTSV |
| | | DVSLIQDVERILDYSLCQETWSKIRAGLPISPVDLSYLAPKNPGTG |
| | | PAFTIINGTLKYFETRYIRVDIAAPILSRMVGMISGTTTEAELWDD |
| | | WAPYEDVEIGPNGVLRTSSGYKFPLYMIGHGMLDSDLHLSSKAQ |
| | | VFEHPHIQDAASQLPDDESLFFGDTGLSKNPIELVEGWFSSWKSSI |
| | | ASFFFIIGLIIGLFLVLRVGIHLCIKLKHTKKRQIYTDIEMNRLGK |
| 78 | OKT3 | MEAPAQLLFLLLLWLPDTTGQVQLQQSGAELARPGASVKMSCK |
| | (VH- | ASGYTFTRYTMHWVKQRPGQGLEWIGYINPSRGYTNYNQKFKD |
| | VL)- - | KATLTTDKSSSTAYMQLSSLTSEDSAVYYCARYYDDHYCLDYW |
| | CD8a- | GQGTTLTVSSGGGGSGGGGSGGGGSQIVLTQSPAIMSASPGEK |
| | VSVG | VTMTCSASSSVSYMNWYQQKSGTSPKRWIYDTSKLASGVPAHF |
| | (K47Q/ | RGSGSGTSYSLTISGMEAEDAATYYCQQWSSNPFTFGSGTKLEIN |
| | R354A) | KPTTTPAPRPPTPAPTIASQPLSLRPEACRPAAGGAVHTRGLMKC |
| | | LLYLAFLFIGVNCKFTIVFPHNQKGNWKNVPSNYHYCPSSSDLN |
| | | WHNDLIGTALQVKMPQSHKAIQADGWMCHASKWVTTCDFRW |
| | | YGPKYITHSIRSFTPSVEQCKESIEQTKQGTWLNPGFPPQSCGYAT |
| | | VTDAEAVIVQVTPHHVLVDEYTGEWVDSQFINGKCSNYICPTVH |
| | | NSTTWHSDYKVKGLCDSNLISMDITFFSEDGELSSLGKEGTGFRS |
| | | NYFAYETGGKACKMQYCKHWGVRLPSGVWFEMADKDLFAAA |
| | | RFPECPEGSSISAPSQTSVDVSLIQDVERILDYSLCQETWSKIRAGL |
| | | PISPVDLSYLAPKNPGTGPAFTIINGTLKYFETRYIRVDIAAPILSR |
| | | MVGMISGTTTEAELWDDWAPYEDVEIGPNGVLRTSSGYKFPLY |
| | | MIGHGMLDSDLHLSSKAQVFEHPHIQDAASQLPDDESLFFGDTG |
| | | LSKNPIELVEGWFSSWKSSIASFFFIIGLIIGLFLVLRVGIHLCIKLK |
| | | HTKKRQIYTDIEMNRLGK |
| 79 | OKT3 | MEAPAQLLFLLLLWLPDTTGQVQLQQSGAELARPGASVKMSCK |
| | (VH-VL) | ASGYTFTRYTMHWVKQRPGQGLEWIGYINPSRGYTNYNQKFKD |
| | GGGGS | KATLTTDKSSSTAYMQLSSLTSEDSAVYYCARYYDDHYCLDYW |
| | x3 | GQGTTLTVSSGGGGSGGGGSGGGGSQIVLTQSPAIMSASPGEK |
| | Prot | VTMTCSASSSVSYMNWYQQKSGTSPKRWIYDTSKLASGVPAHF |
| | | RGSGSGTSYSLTISGMEAEDAATYYCQQWSSNPFTFGSGTKLEIN |
| | | GGGGSGGGGSGGGGS |
| 80 | OKT3 | atggaggcccccgcccaactcctcttcctcctgttgctctggctgccggacaccaccggtcaagtgcag |
| | (VH-VL) | ctgcagcaatcaggggccgagctcgccaggcctggcgcatcagttaaaatgtcatgtaaggcatcag |
| | GGGGS | ggtacacctttacaagatacacaatgcactgggtgaagcagaggcccggtcagggtctggaatggatc |
| | x3 | ggctatatcaatcctagcagaggttatacaaactacaatcaaaaatttaaggataaagccacattgacaa |
| | DNA | ctgacaagagtagttccactgcttatatgcagctgagcagcctcacgtccgaggacagtgcagtttatta |
| | | ttgtgcgcgatattacgacgaccattattgcttggattactggggacagggcacgacgctcacagtcagt |
| | | tctggcggtgggggcagtggggggggtgggtctggaggcggaggctcccagatagtcttgacgcag |
| | | tcaccggctataatgtcagcaagtcctggagagaaagtcacaatgacttgcagtgcgtccagtagtgtta |
| | | gttacatgaactggtaccagcaaaaaagtggtacaagccccaagagatggatctacgacaccagcaa |
| | | gctggcaagtggtgtaccagcgcattttcgaggctctgggagtgggacgtcttactctctcacgattagc |
| | | ggcatggaagcggaagacgcggcaacatattactgtcaacaatggtcttccaatcctttcacttttggttc |
| | | aggcacaaagctcgagataaatggcggtgggggcagtggggggggtgggtctggaggcggagg |
| | | ctcc |
| 81 | α-helical | (EAAAK)n |
| | linker | |

Having now generally described the invention, the same will be more readily understood through reference to the following examples, which are provided by way of illustration and are not intended to be limiting of the present invention.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1. Construction of CD3-Specific Lentiviral Pseudotypes for Targeted Delivery of erbB-2-CAR into T Cells

Significant time and effort have been invested to design and construct novel lentiviral pseudotyping receptors that recognize mouse or human CD3 and facilitate entry and fusion of the viral particles to T cells. By fusing the viral membrane with the endosome membrane of the T cell, the contents of the viral particle are brought into the cytoplasm of the T cell.

The vesicular stomatitis virus glycoprotein (VSVG) is a Class III viral fusion receptor. Both pre- and post-fusion states of the receptor are trimeric. Each receptor monomer consists of three distinct domains: fusion domain (FD), Pleckstrin homology domain (PHD), and central domain (CD). Interaction of residues within the central domains of each receptor facilitates formation of the of the trimer complex. Residues within the central domain also designate its specificity by facilitating binding of VSVG to the cellular LDL receptor.

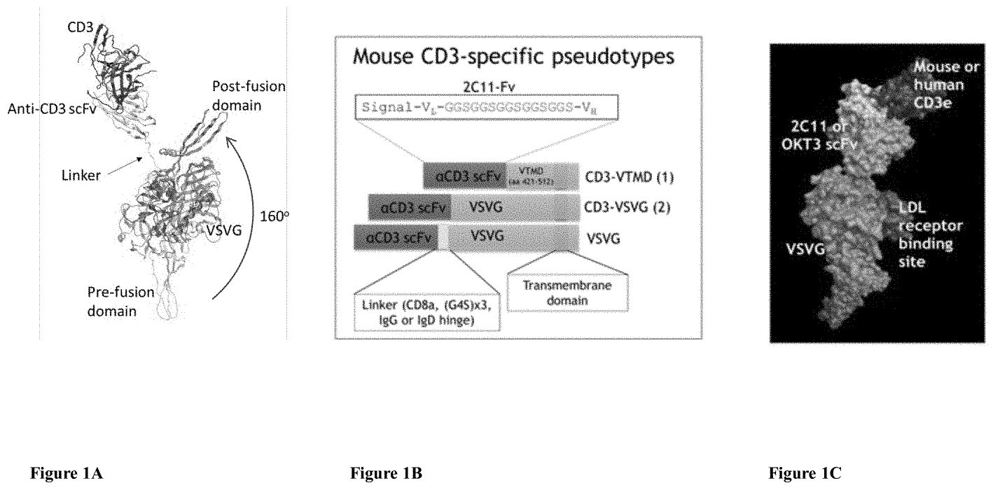

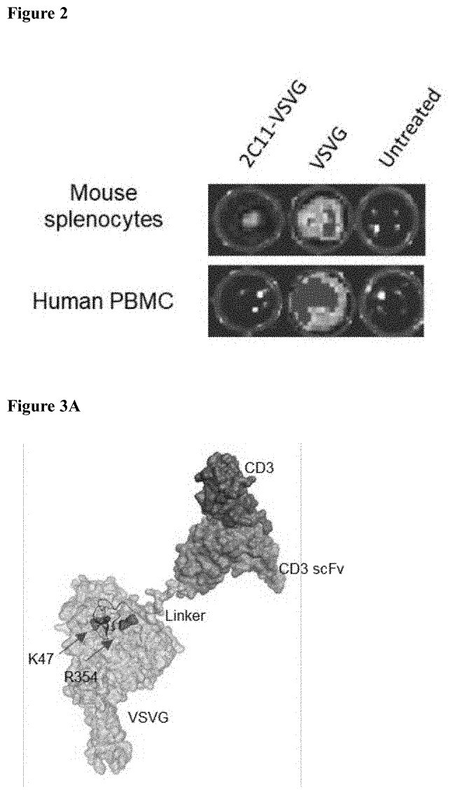

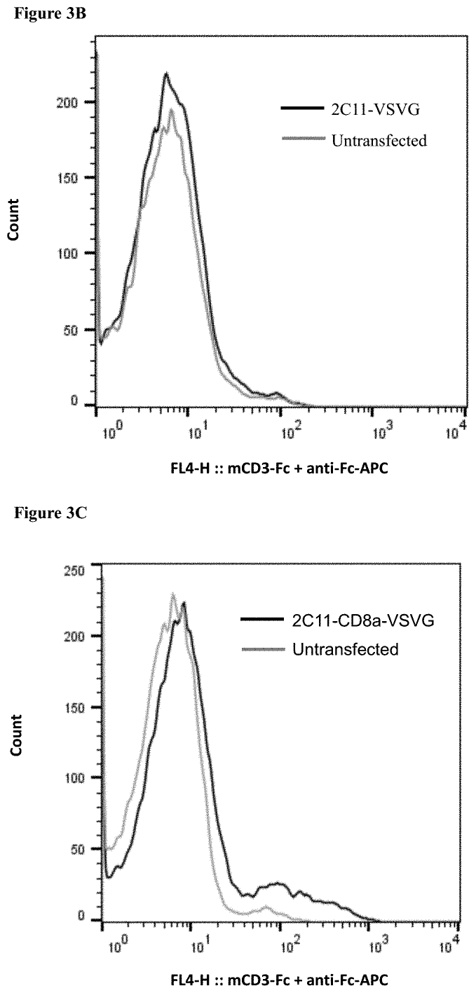

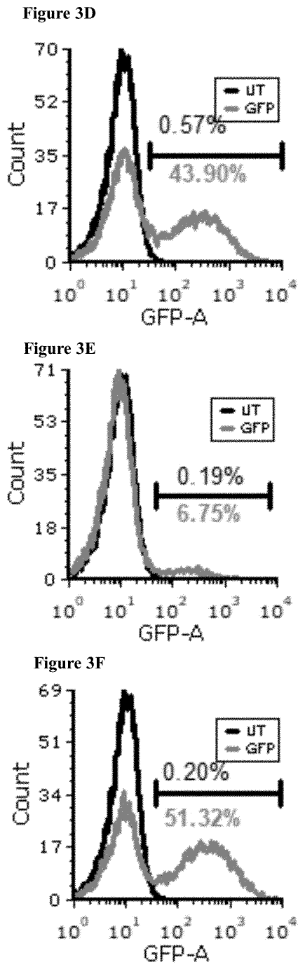

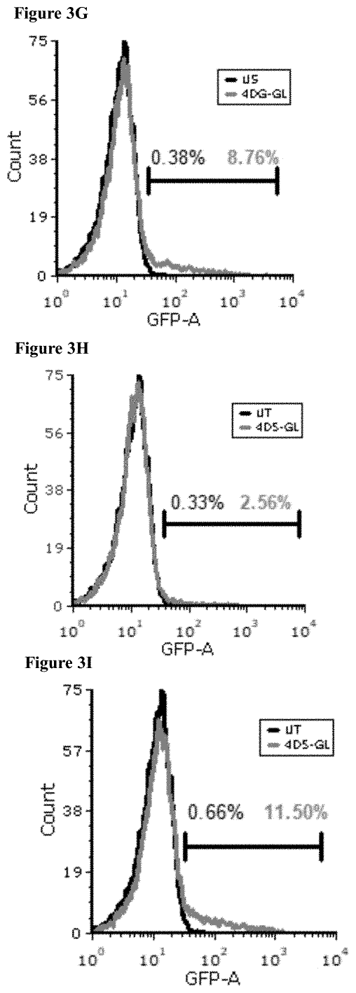

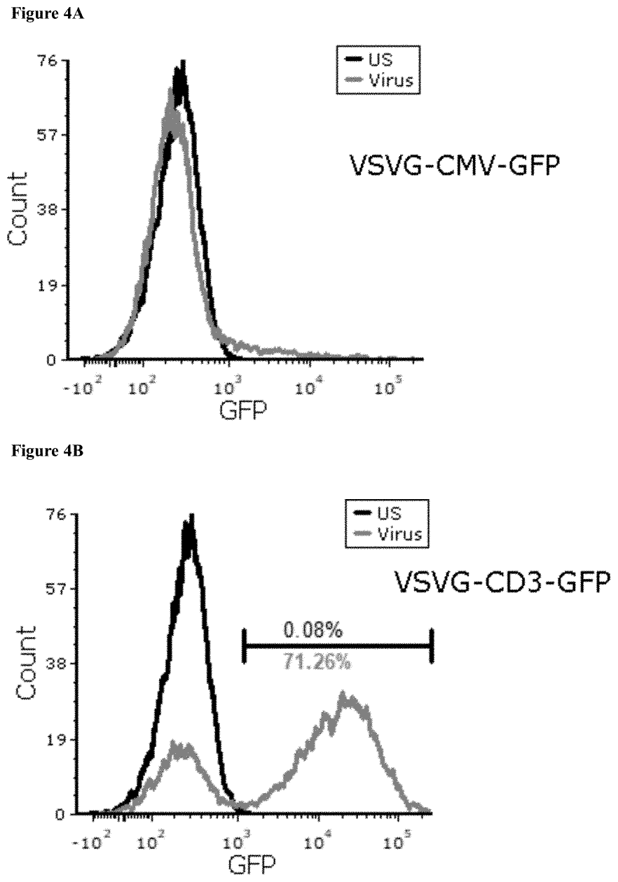

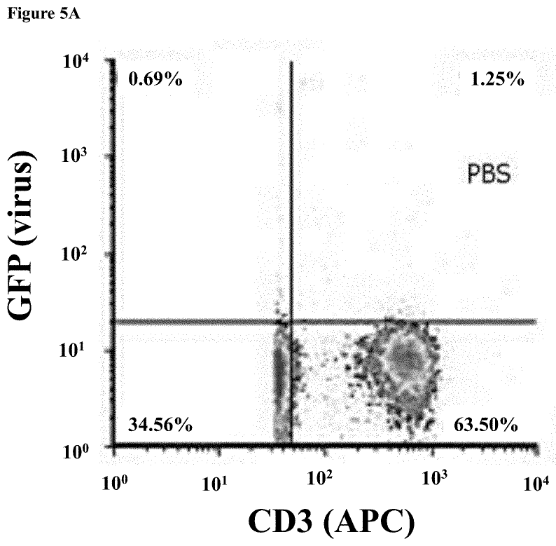

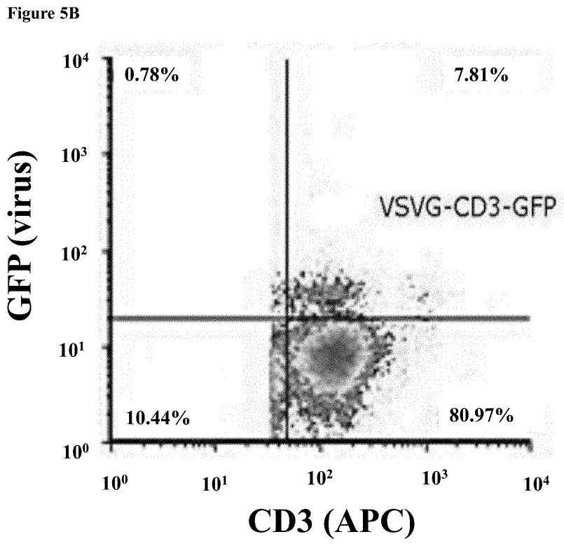

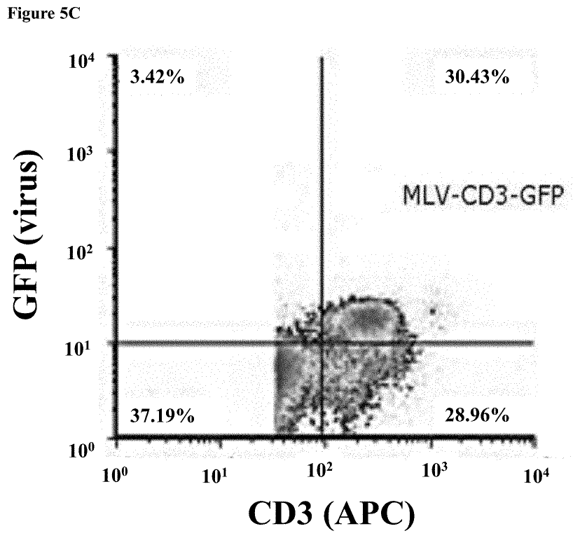

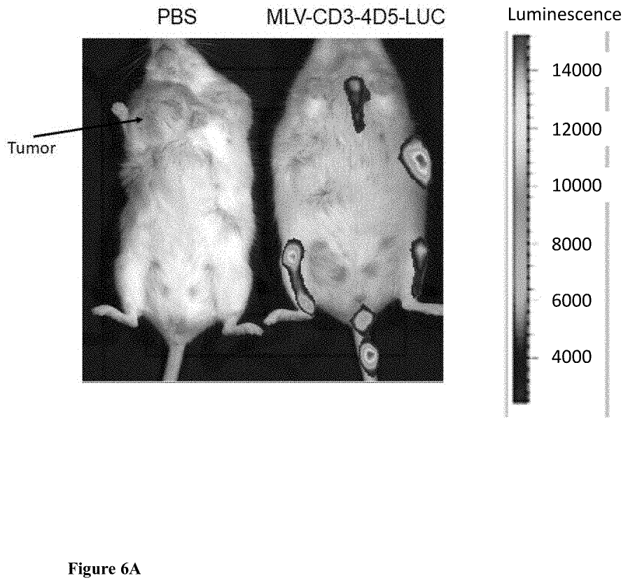

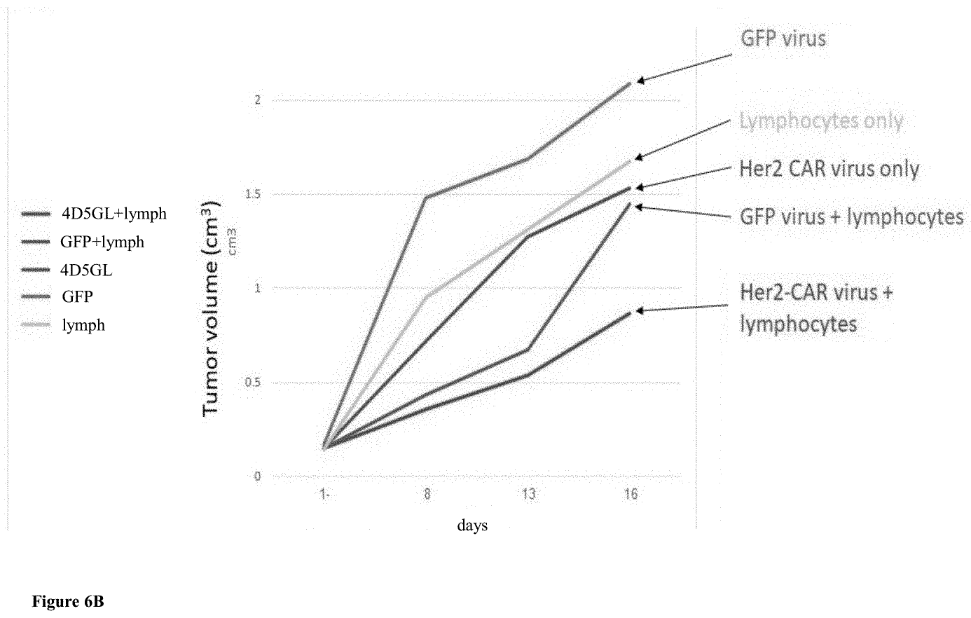

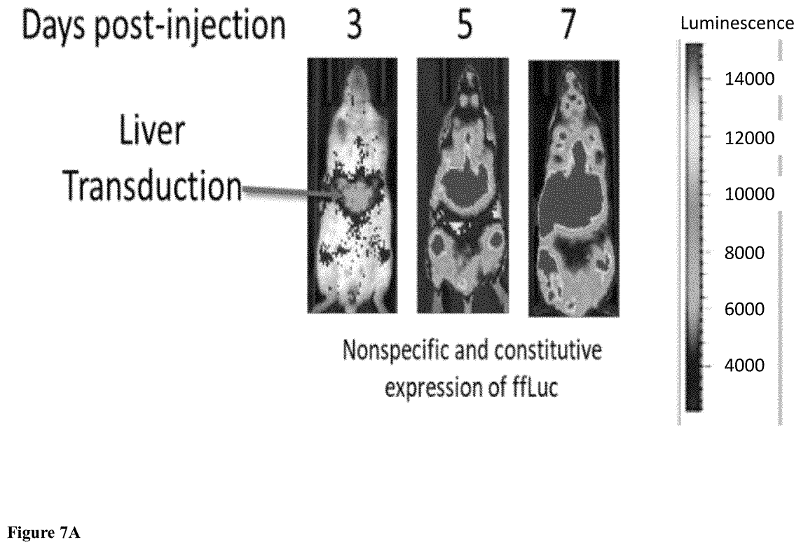

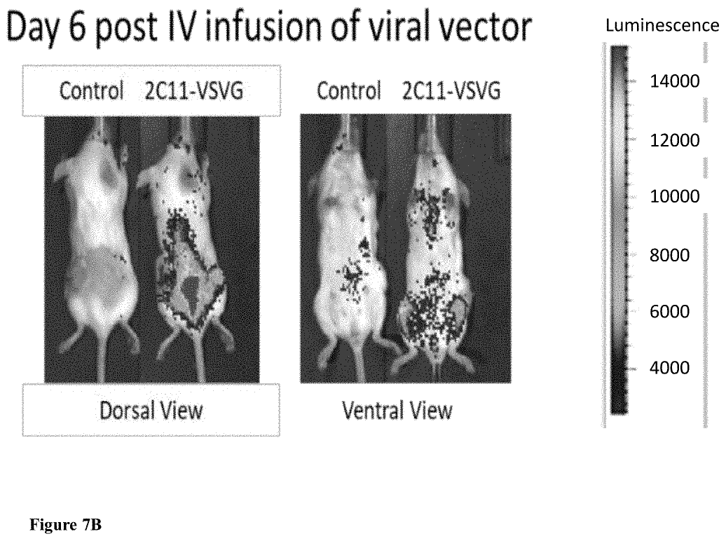

Viral fusion and entry into the host cells is normally initiated by binding of VSVG to cellular LDL receptor (LDLR). Subsequently, the viral particles are engulfed by the host cell into endosomes where the low pH environment triggers a dramatic structural rearrangement of VSVG to facilitate fusion of the viral and cellular membranes. During this structural transition, the downward-facing fusion domains of each VSVG monomer mobilize, turning 160 degrees, to interact with the endosome membrane before transitioning back to their downward facing orientation (FIG. 1A). During this process, terminal hydrophobic loops within the fusion domains interact with the host cell membrane to trigger the fusion process. Fusion of the viral and endosomal membranes allows the viral capsid to exit the endosome and enter the cytoplasm. The capsid is then disintegrated thus releasing the contents of the virus.

As LDLR is expressed on a wide variety of cells, unmodified VSVG will bind to numerous cells creating off target effects when infection of only a specific cell type is desired. Although the LDLR binding domain can be mutated (see below), interaction with a surface protein is needed to facilitate endosomal entry. However, redirection of the specificity of VSVG toward T cells can be achieved through N-terminal fusion of a CD3-specific single chain Fv domain derived from an antibody sequence. The scFv domain initiates binding of the viral particle to cell surface CD3. This event initiates endocytosis of the viral particle into endosomes. The acidic environment within the endosomes triggers reconfiguration of VSVG from a pre-fusion configuration to its post-fusion form facilitating virus-host cell membrane fusion and delivery of molecules within the virus particle.

To this end, chimeric pseudotyping receptors were designed containing a single chain antibody variable fragment (scFv) that recognizes mouse (2C11—SEQ ID NO:13 or 500A2—SEQ ID NO: 54) or human (OKT3—SEQ ID NO: 52) CD3 fused at its C-terminus to a VSVG extracellular domain (SEQ ID NO: 59) (FIG. 1C). The entire molecule is anchored in the viral lipid bilayer by VSVG's transmembrane domain although other transmembrane domains can be employed Additionally, the scFv can be fused to a truncated VSVG consisting of the C-terminal 90 amino acids. This truncated protein contains part of the extracellular domain the transmembrane domain of VSVG and its cytoplasmic tail (VTMD—SEQ ID NO: 18). Expression of this truncated protein has been reported to enhance viral fusion when the full-length protein is also present. It can also be expressed without the scFV.

VSVG's natural receptor, the LDL receptor, is expressed on a wide variety of cells, and this binding may cause nonspecific transduction events. Point mutation within the LDL receptor interacting domain of VSVG can also be made in order to reduce binding to LDL receptor (FIG. 3A). A variety of peptide linkers were tested for connecting the scFV to VSVG (FIG. 1B).

### Example 2. Specificity of Infection with Lentiviral Vectors Expressing Pseudotypes

Lentiviral vectors (LV) expressing pseudotypes prepared as explained in Example 1 and further encoding a firefly luciferase (ffLuc) reporter gene were tested in vitro to assess their specificity in transducing mouse splenocytes and human peripheral blood mononuclear cells (PBMC). BALB/c mouse splenocytes and human PBMCs were seeded in a 96-well plate at a density of 10′ cells in 100 μL per well. 2C11-VSVG-pseudotyped lentiviral particles encoding ffLuc and GFP were then added to the cells (25 μL of lentiviral suspension at 107 IU/mL). Bioluminescence signal was then measured in an IVIS 200 instrument (PerkinElmer) following the addition of 10 μg D-luciferin (10 μL of a 1 mg/mL solution).

As expected, VSVG alone bound indiscriminately to all cells. As B cells and T cells of both mice and humans express LDLR, the VSVG expressing virus is internalized by binding of its native receptor. In contrast, the mouse-specific anti-CD3 scFv (2C11) fused with full-length VSVG (2C11-VSVG—SEQ ID NO: 15) was found to be specific in transducing mouse CD3+ T cells (FIG. 2). Importantly, the chimeric receptor did not infect the human cells (FIG. 2) and also did not infect B cells within the splenocyte population (data not shown). This result is highly unexpected, as VSVG is still present and would have been expected to still bind its canonical target LDLR. The fact that the chimeric molecule cannot bind human cells or B cells indicates that the presence of the scFV is occluding the LDLR binding site (see FIG. 3A). This result indicated that the scFV has the added benefit of diminishing the necessity to introduce point mutations into the LDLR binding domain to prevent VSVG binding to its ubiquitous cognate receptor along with directing transduction exclusively toward the intended CD3+target cells.

It should also be noted that while addition of the anti-CD3 scFV conferred cell-type specificity it greatly reduced overall infection levels. VSVG alone infected a far greater number of cells and the expression level of luciferase in those cells was considerably higher than those successfully infected by the virus with the chimeric receptor (FIG. 2). It was thus necessary to improve infection of T cells without losing the specificity unexpectedly provided by the scFV.

### Example 3. Modifications of 2C11-VSVG Pseudotyping Receptor

In order to improve the affinity and efficacy of the CD3-binding pseudotyping receptor, modifications hypothesized to minimize steric hindrances between the CD3-specific scFv and the VSVG domains were investigated. It was hypothesized that optimal function of the scFv-VSVG chimeric receptor necessitates the presence of a linker between these domains (scFv-linker-VSVG) to minimize functional interference with the dynamic conformational transition of VSVG during the membrane fusion process. The linker functions to distance the scFv domain from the fusion domain during the structural reconfiguration process of VSVG, which entails movement of the fusion domain 160 degrees from its pre-fusion state (FIG. 1A). Moreover, distancing of the scFv and VSVG domains with a linker minimizes hindrance between the VSVG monomers to allow their assembly into a trimeric pre-fusion configuration.

A composite structure of the CD3 scFv-VSVG fusion protein with a linker is shown in FIG. 3A. The proximity of the scFV to the LDLR binding site is apparent. The first linker tested was the CD8a stalk domain (SEQ ID NO: 5), and to this end a 2C11-CD8a-VSVG fusion (SEQ ID NO: 16) was generated.

To test the binding of the pseudotyping receptor, human 293 cells were transfected with either a vector encoding a direct fusion of the scFv to VSVG or a vector encoding the CD8a linker containing fusion protein. The cells were then contacted with mouse CD3-Fc fusion protein and then stained with a fluorescent anti-Fc antibody. Control 293 cells were not transfected. Inclusion of a CD8a stalk linker between the VSVG and CD3 scFv enhanced binding to CD3-Fc peptide (FIG. 3B-C). Additional linkers including the IgG Hinge, IgD Hinge, and GGGS flexible linker are also tested for enhanced binding to 293 cells expressing CD3.

This was further tested using the human anti-CD3 OKT3 scFv. 293T cells were transfected to produce GFP expression vector-carrying virus particles coated with different envelope proteins: OKT3-VSVG (SEQ ID NO: 64), OKT3-CD8a-VSVG (SEQ ID NO: 65) and VSVG alone. The 293T supernatant containing virus was supplemented with IL-2 or IL-7/15 and polybrene. Activated lymphocytes were transduced with these viruses. This was done by centrifugation for 90 minutes at 800 g followed by overnight incubation. The next day the viruses were replaced by fresh media. GFP expression was analyzed at least 3 days after transduction. As expected, VSVG alone produced robust expression with ˜44% of the cells GFP positive (FIG. 3D). In contrast, the scFv directly linked to VSVG produced greatly reduced transduction with only ˜7% of the cells GFP positive (FIG. 3E). The inclusion of the CD8a linker had a dramatic effect. Over 50% of the cells transduced with the linker construct were GFP positive (FIG. 3F). Thus, not only did the inclusion of the linker solve the problem of the steric hindrance caused by the scFv, but the total effect of the linker construct was even greater than VSVG alone. This increase is even without the benefit of targeting only to T cells as in this test only lymphocytes were present, so this improvement over VSVG alone is only part of the improvement provided by the linker construct.

This transduction was then repeated using a CAR directed against the HER2 protein (also expressing GFP), instead of the GFP plasmid only. Again, 293T cells were transfected to produce the CAR expressing vector-carrying virus particles using different envelopes: OKT3-VSVG (SEQ ID NO: 64), OKT3-CD8a-VSVG (SEQ ID NO: 65) and VSVG alone. Supernatant supplemented with IL-2 was used to transduce activated lymphocytes. Transduction percentage was assessed by GFP expression. VSVG enveloped viruses gave an expression of 8.76% (FIG. 3G), OKT3-VSVG transduced cells expressed 2.56% (FIG. 3H) and OKT3-CD8a-VSVG virus increased the expression to 11.5% (FIG. 3I).

### Example 4. Expression of CAR Under CD3 Promoter

An alternative or parallel approach to enhance specificity of expression in T cells is the incorporation of a CD3 promoter (SEQ ID NO: 17) to drive the expression of lentiviral transgenes. Lentivirus pseudotype was generated by transfecting 293T cells with 4 different plasmids expressing the virus envelope (VSVG), the gag-pol (RRE), the reverse transcriptase (REV) and the expression plasmid expressing either GFP or the 4D5 based CAR under the control of the CD3 promoter. A vector with GFP under the control of the CMV promoter was used as a control. Viral particles were collected from cell medium at 48 h and 72 h. Viral particles were concentrated ×100 using the LentiX protocol (Clontech). The virus is then aliquoted and kept at −80° C. until used.

Primary human T cells were grown in culture and then contacted with the virus expressing GFP under the control of the CD3 promoter or the CMV promoter. As can be seen in FIGS. 4B, CD3 promoter-driven expression of GFP was very high in human T cells following transduction with VSVG-pseudotyped lentiviral vectors. In contrast, CMV-GFP virus did not produce GFP in the T cells (FIG. 4A).

### Example 5. Specificity of In-Vivo T Cell Transduction Using Lentiviral Vectors

To further evaluate the utility of CD3 promoter driven expression, lentiviral vectors expressing murine leukemia virus (MLV) pseudotypes and encoding GFP under the control of a CD3 promoter (SEQ ID NO: 17) were generated (FIG. 5A-C).

Preliminary experiments demonstrating in vivo transduction of endogenous mouse T cells were performed in an FVB Her2 transgenic mouse model. VSVG-(nonspecific) or MLV-pseudotyped lentivectors with the CD3 promoter controlling transcription of GFP were compared. PBS alone was used as a negative control. Virus pseudotype was prepared as described hereinabove. 200 μl of virus was injected intravenously to FVB Her2 transgenic mice bearing tumors. One week following intravenous infusion of lentiviral particles, the mice were bled and GFP expression was assayed in the PBMC population. The PBMCs were stained for CD3 and GFP expression in these cells was examined. The gate for negative GFP and positive CD3 expression was set based on the PBS treated sample (FIG. 5A). When VSVG was used as the fusion molecule the plasmid with CD3 promoter-GFP was able to enter cells and GFP was detected. Importantly GFP expression was almost exclusively observed in the CD3 cells (FIG. 5B). Similar results were observed when MLV was used (FIG. 5C).

Next, an MLV-pseudotyped virus encoding a Her2-specific CAR and ffLuc and GFP under the control of the CD3 promoter was generated. The Her2-binding domain of this CAR is an scFv derived from the anti-Her2 antibody 4D5. The virus was injected intravenously (iv) into tumor bearing FVB Her2 transgenic mice and detected by bioluminescence imaging 7 days later. Expression of luciferase can be detected in lymphatic organs, mainly spleen and bone marrow, indicating in vivo transduction of T cells (FIG. 6A).

Next, naïve mouse splenocytes were activated in vitro using IL-7, αCD3 and αC28 for 72 h. The splenocytes were then resuspended in the different viruses (MLV pseudotype) expressing either GFP or the 4D5-GFP-ffLUC CAR under the control of the CD3 promoter and injected immediately intratumorally. Splenocytes not contacted with any virus and the viruses alone without the splenocytes were also injected as controls.

Tumor volume was measured with a calliper for the next 16 days. Tumor volume increases steadily in mice administered GFP expressing control virus (FIG. 6B green line). Lymphocytes that were not infected (yellow line) and those infected with GFP (blue line) were comparable as would be expected and both produced a reduction in overall tumor size (FIG. 6B). Importantly, administration of virus containing the CAR vector under control of CD3 promoter without lymphocytes (purple line) had a significant effect on tumor size reduction, and indeed was comparable to the lymphocytes alone (FIG. 6B). This indicate that the virus can infect T cells in vivo and induce an antitumor effect. Finally, lymphocytes infected with virus encoding the CAR under CD3 promoted control (red line) were by far the most effective in reducing tumor volume (FIG. 6B).

### Example 6. In Situ Transduction with CD3-Pseudotyped Lentiviral Vectors

Similar experiments were next performed with the chimeric pseudotyping vector. To determine the efficacy of in situ transduction, suspensions of VSVG or 2C11-VSVG pseudotyped lentivirus expressing ffLuc-GFP (107 IU/100 uL) were injected intravenously. Transduction was monitored by bioluminescence imaging. As shown in FIG. 7A, transduction with VSVG-pseudotyped lentivirus demonstrated a dominant initial signal in the liver at day 3 followed by a steady systemic increase by day 7. This pattern of transduction is consistent with tropism of VSVG lentivectors for LDL receptors expressed on the surface of liver cells and many other cells. In contrast, 2C11-VSVG pseudotyped lentivirus infusion exhibited a drastically different pattern of ffLuc gene expression consistent with T cell distribution (FIG. 7B). At day 6 post-infusion, bioluminescent signal was evident throughout the skeletal system consistent with bone marrow-localized transduction of T cells. No activity was detected in the liver indicating not only CD3 specificity of the scFv, but also blocking of VSVG binding to its native receptor.

### Example 7. Preparation of VSVG-OKT3 and VSVG-CD8a-OKT3 Pseudotyped LV

Pseudotyped fusion protein specific to human CD3 comprising OKT3-VSVG or OKT3-CD8a-VSVG is prepared. The OKT3-VSVG fusion protein comprises amino acid sequence SEQ ID NO: 11 or 64 and the OKT3-CD8a-VSVG comprises amino acid sequence SEQ ID NO: 12 or 65. The pseudotyped lentivirus comprising the proteins are generated and tested. The plasmids encoding OKT3-VSVG or OKT3-CD8a-VSVG are transfected to HEK293 cells along with the transgene-encoding and the gag-pol and rev packaging plasmids to produce the respective pseudotyped lentivirus. Additional pseudotyped fusion proteins using other linkers, e.g., linkers having amino acid sequence SEQ ID NOs: 7, 9, 20, 22, 24, and 26-29 are also generated. The resulting fusion proteins have amino acid sequences SEQ ID NOs: 39-51 and 66-78. Plasmids encoding the fusion proteins are produced as described and transfected to produce the respective pseudotyped lentivirus. The lentiviruses are tested for their ability to infect specifically CD3+ T-cells and not other cells that express LDLR. The relative fusion capacity with each different linker is assessed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A pseudotyped virus or virus-like particle comprising a fusion protein comprising a vesicular stomatitis virus envelope glycoprotein (VSVG) extracellular domain (ECD) or a fragment or an analog thereof capable of fusing with a cellular membrane linked to a polypeptide comprising an antigen binding domain specific to cluster of differentiation 3 (CD3).

2. (canceled)

3. The pseudotyped virus or virus-like particle according to claim 1, wherein said antigen binding domain specific to CD3 is OKT3.

4. The pseudotyped virus or virus-like particle according to claim 1, wherein at least one of:
a. said polypeptide is a single variable fragment (scFv) of an antibody that specifically binds CD3;
b. said ECD comprises or consists of SEQ ID NO: 59;
c. said pseudotyped virus comprises full-length VSVG;
d. said pseudotyped virus comprises full-length VSVG comprising a signal peptide and comprising or consisting of SEQ ID NO: 1;
e. said pseudotyped virus comprises full-length VSVG devoid of a signal peptide and comprising or consisting of SEQ ID NO: 60;
f. said pseudotyped virus comprises a truncated VSVG lacking an intracellular domain;
g. said pseudotyped virus comprises a truncated VSVG lacking an intracellular domain and comprises a transmembrane domain from a protein other than VSVG;
h. said pseudotyped virus comprises a truncated VSVG comprising or consisting of amino acid sequence SEQ ID NO: 18;
i. said VSVG comprises a mutation that decreases binding to low-density lipoprotein (LDL) receptor;
j. said VSVG comprises a mutation that decreases binding to low-density lipoprotein (LDL) receptor selected from mutation of K47 of VSVG, mutation of R354 of VSVG or both, optionally wherein said K47 is mutated to A, G or Q, said R354 is mutated to A or G or wherein said VSVG analog comprises or consists of an amino acid sequence selected from SEQ ID NO: 33, 35 and 37.

5. (canceled)

6. (canceled)

7. (canceled)

8. (canceled)

9. (canceled)

10. (canceled)

11. The pseudotyped virus or virus-like particle according to claim 1, wherein said VSVG ECD or fragment or analog thereof comprises or consists of an amino acid sequence selected from SEQ ID NO: 1, 18, 33, 35, 37, 59, and 60.

12. The pseudotyped virus or virus-like particle according to claim 1, wherein at least one of:
a. said polypeptide is linked to an N-terminus of said VSVG ECD, fragment or analog thereof;
b. said VSVG, analog or fragment thereof and the polypeptide are linked via a linker;
c. said VSVG, analog or fragment thereof and the polypeptide are linked via a peptide linker;
d. said VSVG, analog or fragment thereof and the polypeptide are linked via a peptide linker comprising at least 10 amino acids;
e. said VSVG, analog or fragment thereof and the polypeptide are linked via a rigid linker; and
f. said VSVG, analog or fragment thereof and the polypeptide are linked via a linker selected from a CD8a stalk, an IgG hinge, an IgD linker and a GGGGS linker, wherein the GCGCS linker comprises 2 to 5 repetitions of amino acid sequence GGGGS.

13. (canceled)

14. (canceled)

15. (canceled)

16. (canceled)

17. The pseudotyped virus or virus-like particle according to claim 1, wherein said VSVG, analog or fragment thereof and the polypeptide are linked via a linker and the linker comprises or consists of an amino acid sequence selected from SEQ ID NO: 5, 7, 9, 20, 22, 24, and 26-29.

18. The pseudotyped virus or virus-like particle according to claim 1, wherein said fusion protein comprises or consists of an amino acid sequence selected from SEQ ID NO: 11, 12, 39-51 and 64-78.

19. (canceled)

20. The pseudotyped virus or virus-like particle according to claim 1, wherein:
a. the virus is selected from lentivirus, adenovirus, retrovirus, Epstein-Barr virus, herpes simplex virus 1 (HSV1), a myxoma virus, a reovirus, a poliovirus, a vesicular stomatitis virus (VSV), and a measles virus (MV);
b. said pseudotyped virus or virus-like particle further comprises a membranal protein of interest or a nucleic acid molecule encoding for said membranal protein of interest;
c. said pseudotyped virus or virus-like particle further comprises a chimeric antigen receptor (CAR) or a T-cell receptor or a nucleic acid molecule encoding for said CAR or T-cell receptor;
d. said pseudotyped virus or virus-like particle further comprises a CAR that binds specifically to a tumor associated antigen or a nucleic acid molecule encoding for said CAR;
e. said pseudotyped virus or virus-like particle further comprises a CAR that binds specifically to a protein selected from ErbB2/Her2, CD19, CD20, CD22, CD30, CD33, CD38, CD40, CD123, CD133, CD138, CD5, CD7, APRIL, BCMA, CEA, MUCI, EGFR, GD2, Mesothelin, and CDK4 or a nucleic acid molecule encoding for said CAR;
f. comprising a homotrimer of said fusion protein;
g. comprising a nucleic acid molecule comprising a regulatory element operably linked to an open reading frame encoding a membranal protein of interest or CAR and wherein said regulatory element induces transcription in T cells;
h. comprising a nucleic acid molecule comprising a T cell active promoter selected from a CD3, CD4 and CD8 promoter operatively linked to an open reading frame encoding a membranal protein of interest or CAR; or
i. a combination thereof.

21. (canceled)

22. (canceled)

23. (canceled)

24. (canceled)

25. (canceled)

26. (canceled)

27. (canceled)

28. (canceled)

29. (canceled)

30. A fusion protein comprising a vesicular stomatitis virus envelope glycoprotein (VSVG) extracellular domain or a fragment or an analog thereof capable of fusing with a cellular membrane linked at its N-terminus to a polypeptide comprising an antigen binding domain specific to cluster of differentiation 3 (CD3).

31. (canceled)

32. The fusion protein according to claim 30, wherein said antigen binding domain specific to CD3 is OKT3.

33. The fusion protein according to claim 30, wherein at least one of:
a. said polypeptide is a single variable fragment (scFv) of an antibody that specifically binds CD3;
b. said ECD comprises or consists of SEQ ID NO: 59;
c. said pseudotyped virus comprises full-length VSVG;
d. said pseudotyped virus comprises full-length VSVG comprising a signal peptide and comprising or consisting of SEQ ID NO: 1;
e. said pseudotyped virus comprises full-length VSVG devoid of a signal peptide and comprising or consisting of SEQ ID NO: 60;
f. said pseudotyped virus comprises a truncated VSVG lacking an intracellular domain;
g. said pseudotyped virus comprises a truncated VSVG lacking an intracellular domain and comprises a transmembrane domain from a protein other than VSVG;
h. said pseudotyped virus comprises a truncated VSVG comprising or consisting of amino acid sequence SEQ ID NO: 18;
i. said VSVG comprises a mutation that decreases binding to low-density lipoprotein (LDL) receptor;
j. said VSVG comprises a mutation that decreases binding to low-density lipoprotein (LDL) receptor selected from mutation of K47 of VSVG, mutation of R354 of VSVG or both, optionally wherein said K47 is mutated to A, G or Q, said R354 is mutated to A or G or wherein said VSVG analog comprises or consists of an amino acid sequence selected from SEQ ID NO: 33, 35 and 37.

34. (canceled)

35. (canceled)

36. (canceled)

37. (canceled)

38. (canceled)

39. (canceled)

40. The fusion protein according to claim 30, wherein said VSVG extracellular domain, fragment or analog thereof comprises or consists of an amino acid sequence selected from SEQ ID NO: 1, 18, 33, 35, 37, 59 and 60.

41. The fusion protein according to claim 30, wherein at least one of:
a. said VSVG, analog or fragment thereof and the polypeptide are linked via a linker;
b. said VSVG, analog or fragment thereof and the polypeptide are linked via a peptide linker;
c. said VSVG, analog or fragment thereof and the polypeptide are linked via a peptide linker comprising at least 10 amino acids;
d. said VSVG, analog or fragment thereof and the polypeptide are linked via a rigid linker;
e. said VSVG, analog or fragment thereof and the polypeptide are linked via a linker selected from a CD8a stalk, an IgG hinge, an IgD linker and a GGGGS linker, wherein the GCGCS linker comprises 2 to 5 repetitions of amino acid sequence GGGGS; and
f. said fusion protein is in a form of a homotrimer.

42. (canceled)

43. (canceled)

44. (canceled)

45. The fusion protein according to claim 41, wherein said VSVG, analog or fragment thereof and the polypeptide are linked via a linker and the linker comprises or consists of an amino acid sequence selected from SEQ ID NO: 5, 7, 9, 20, 22, 24, and 26-29.

46. The fusion protein according to claim 30, comprising or consisting of an amino acid sequence selected from SEQ ID NO: 11, 12, 39-51 and 64-78.

47. (canceled)

48. (canceled)

49. A nucleic acid molecule encoding a fusion protein according to claim 30.

50. A pharmaceutical composition comprising a pseudotyped virus or virus-like particle according to claim 1, and a pharmaceutically acceptable carrier, excipient or adjuvant.

51. (canceled)

52. A method of treating cancer in a subject in need thereof comprising administering a therapeutically effective amount of a pharmaceutical composition according to claim 50 to said subject, thereby treating cancer.

53. The method according to claim 52, wherein at least one of:
a. said administering is systemically or intratumorally administering;
b. said treating comprises infecting T-cells with said pseudotyped virus;
c. said method further comprises providing a sample comprising T cells, contacting said sample with said pseudotyped virus or said pharmaceutical composition under conditions sufficient for infection of said T cells with said pseudotyped virus, and administering said infected T cells to said subject;
d. said cancer expresses an antigen and said pseudotyped virus or virus-like particle comprises a CAR that binds specifically to said antigen.

54. (canceled)

55. (canceled)

56. (canceled)

57. A method for expressing a membranal protein of interest in a T-cell, the method comprising contacting said T-cell with a pseudotyped virus or virus-like particle according to claim 1 further comprising said membrane protein of interest, thereby expressing a membranal protein of interest in a T cell, optionally wherein the expression is performed in situ and contacting T-cell with pseudotyped virus comprises administering the pseudotyped virus to a subject.

58. (canceled)

59. (canceled)
